(19) United States
(12) Patent Application Publication
(10) Pub. No.: US 2019/0240252 A1
(43) Pub. Date: Aug. 8, 2019

(54) PHARMACEUTICAL COMPOSITIONS

(71) Applicant: GENZYME CORPORATION, Cambridge, MA (US)

(72) Inventors: Stephen Randall HOLMES-FARLEY, Arlington, MA (US); David J. HARRIS, Lexington, MA (US); Steven C. POLOMOSCANIK, Bedford, MA (US); Adnan SALAMEH, Irvine, CA (US); Bruce SHUTTS, Bolton, MA (US); Richard SILVA, Needham, MA (US); Pradeep K. DHAL, Westford, MA (US); Lynne SOLE, Billerica, MA (US)

(21) Appl. No.: 16/267,238

(22) Filed: Feb. 4, 2019

Related U.S. Application Data
(62) Division of application No. 14/968,170, filed on Dec. 14, 2015, now abandoned.
Application No. 14/968,170 is a continuation of application No. 14/255,368, filed on Apr. 17, 2014, now abandoned.
Application No. 14/255,368 is a continuation of application No. 12/314,554, filed on Dec. 12, 2008, now abandoned.
(60) Provisional application No. 61/006,019, filed on Dec. 14, 2007.

(51) Int. Cl. A61K 31/785; B01J 41/14; A61K 9/20; A61K 9/16; A61K 9/28; A61K 9/14

(52) U.S. Cl. CPC A61K 31/785; B01J 41/14; A61K 9/20; A61K 9/1688; A61K 9/16; A61K 9/2095; Y10T 428/2982; A61K 9/2853; A61K 9/284; A61K 9/282; A61K 9/2031; A61K 9/146; A61K 9/2077

(57) ABSTRACT

The present invention relates to crosslinked polyamine particles and/or pharmaceutical compositions comprising, at least in part, crosslinked polyamine particles and aggregates of such particles (including cured aggregates of crosslinked polyamine particles). The compositions may be in the form of tablets comprising, for example, particles larger than 500 μm, and used for treating patients, for example, patients with hyperphosphatemia.

### CROSS REFERENCE TO RELATED APPLICATION

This application claims the benefit of U.S. Provisional Application No. 61/006,019, filed Dec. 14, 2008, and is incorporated herein by reference in its entirety.

### FIELD OF THE INVENTION

This invention relates to pharmaceutically acceptable compositions and polymers or residues thereof for binding target ions, and more specifically relates to polymer particles for binding target ions.

### BACKGROUND OF THE INVENTION

Hyperphosphatemia frequently accompanies diseases associated with inadequate renal function such as end stage renal disease (ESRD), hyperparathyroidism, and certain other medical conditions. The condition, especially if present over extended periods of time, leads to severe abnormalities in calcium and phosphorus metabolism and can be manifested by aberrant calcification in joints, lungs, and eyes.

Therapeutic efforts to reduce serum phosphate include dialysis, reduction in dietary phosphate, and oral administration of phosphate binders to reduce gastrointestinal absorption. Many such treatments have a variety of unwanted side effects and/or have less than optimal phosphate binding properties, including potency and efficacy. Accordingly, there is a need for compositions and treatments with good phosphate-binding properties and good side effect profiles.

### Definitions

The following definitions apply herein unless otherwise specifically noted:

Aggregate particle: an aggregate particle is a particle that is assembled from, formed from or comprises distinct constituent particles.

d₁₀: the particle size within a distribution of particles where 10 vol. % of the particles have a smaller particle size.

d₅₀: the particle size within a distribution of particles where 50 vol. % of the particles have a particle size that is larger and where 50 vol. % of the particles have a particle size that is smaller.

d₉₀: the particle size within a distribution of particles where 90 vol. % of the particles have a smaller particle size.

Crosslinked polyamine particles: particles comprising at least one crosslinked polyamine for example particles that comprise at least a substantial portion, by weight, of crosslinked polyamine, wherein the substantial portion is at least 50 wt. %, 60 wt. %, 70 wt. %, 80 wt. %, 90 wt. %, 95 wt. %, 98 wt. %, or 99 wt. % as well as 100 wt. %.

Crosslinked polyallylamine particles: particles comprising polyallylamine crosslinked with 7-12 wt. % epichlorohydrin, for example particles that comprise at least a substantial portion, by weight, of crosslinked polyallylamine, wherein the substantial portion is at least 50 wt. %, 60 wt. %, 70 wt. %, 80 wt. %, 90 wt. %, 95 wt. % 98 wt. %, or 99 wt. % as well as 100 wt. %.

### BRIEF SUMMARY OF THE INVENTION

In one aspect, the present invention relates to crosslinked polyamine particles and/or pharmaceutical compositions comprising, at least in part, crosslinked polyamine particles. Compositions can comprise one or more crosslinked polyamines. Several embodiments of the invention are described in further detail as follows. Generally, each of these embodiments can be used in various and specific combinations, and with other aspects and embodiments unless otherwise stated herein.

In addition to the crosslinked polyamine particles of the present invention as described herein, other forms of the crosslinked polyamine particles are within the scope of the invention including pharmaceutically acceptable salts, solvates, hydrates, prodrugs, polymorphs, clathrates, and isotopic variants and mixtures thereof of the crosslinked polyamine particles.

In addition, crosslinked polyamine particles of the invention may have optical centers or chiral centers and the crosslinked polyamine particles of the present invention include all of the isomeric forms of these crosslinked polyamine particles, including optically pure forms, racemates, diastereomers, enantiomers, tautomers and/or mixtures thereof.

In some embodiments, the crosslinked polyamine particles may have a particle size distribution such that greater than 90 vol. % of the crosslinked polyamine particles have a particle size between 250 μm and 4 mm. In some embodiments, the crosslinked polyamine particles may have a particle size distribution where greater than 5 vol. % of the crosslinked polyamine particles has a particle size larger than 500 μm. In some embodiments, the crosslinked polyamine particles have a particle size distribution such that no more than 0 to 20 vol. % of the crosslinked polyamine particles has a particle size smaller than 300 μm. In some embodiments, the crosslinked polyamine particles may have a particle size distribution such that the d₁₀ value is between 250 μm and 750 μm and/or the d₉₀ value is between 900 μm and 1600 μm. In some embodiments, the crosslinked polyamine particles may have a d₅₀ that is between 450 μm and 1100 μm.

In some embodiments, 75 wt. % to 100 wt. % of the crosslinked polyamine particles have a mesh size that is −5/+60. In some embodiments, greater than 5 wt. % of the crosslinked polyamine particles have a mesh size that is +35. In some embodiments, no more than 0 to 20 wt. % of the crosslinked polyamine particles have a mesh size that is -−50. In some embodiments, between 40 wt. % and 60 wt. % of the crosslinked polyamine particles have a mesh size that is −16/+40.

In some embodiments, the invention is, consists essentially of, or comprises crosslinked polyamine particles, a pharmaceutical composition comprising crosslinked polyamine particles or a method for removing a compound or ion, such as a phosphorous-containing compound or a phosphorous-containing ion (e.g., phosphate), from the gastrointestinal tract of an animal by administering an effective amount of crosslinked polyamine particles or a pharmaceutical composition comprising crosslinked polyamine particles wherein the crosslinked polyamine particles comprises polyallylamine crosslinked with from 7-12 wt. % epichlorohydrin, the crosslinked polyallylamine particles having one or more of the particle size characteristics described herein, such as for example, a particle size distribution such that greater than 5 vol. % of the crosslinked polyallylamine particles have a particle size greater than 500 μm, such as between 500 μm and 2 mm.

In some embodiments, the invention is, consists essentially of, or comprises crosslinked polyamine particles, a pharmaceutical composition comprising crosslinked polyamine particles or a method for removing a compound or ion, such as a phosphorous-containing compound or a phosphorous-containing ion (e.g., phosphate), from the gastrointestinal tract of an animal by administering an effective amount of crosslinked polyamine particles or a pharmaceutical composition comprising crosslinked polyamine particles, wherein the crosslinked polyamine particles comprise polyallylamine crosslinked with from 7-12 wt. % epichlorohydrin, the crosslinked polyallylamine particles having a mean gray value of greater than 180.

In some embodiments, the invention is, consists essentially of, or comprises crosslinked polyamine particles, a pharmaceutical composition comprising crosslinked polyamine particles or a method for removing a compound or ion, such as a phosphorous-containing compound or a phosphorous-containing ion (e.g., phosphate), from the gastrointestinal tract of an animal by administering an effective amount of crosslinked polyamine particles or a pharmaceutical composition comprising crosslinked polyamine particles, wherein the crosslinked polyamine particles comprise polyallylamine crosslinked with from 7-12 wt. % epichlorohydrin, the crosslinked polyallylamine particles comprising 2 or more constituent particles comprising polyallylamine crosslinked with 7-12 wt. % epichlorohydrin.

In some embodiments, the invention is, consists essentially of, or comprises crosslinked polyamine particles, a pharmaceutical composition comprising crosslinked polyamine particles or a method for removing a compound or ion, such as a phosphorous-containing compound or a phosphorous-containing ion (e.g., phosphate), from the gastrointestinal tract of an animal by administering an effective amount of crosslinked polyamine particles or a pharmaceutical composition comprising crosslinked polyamine particles, wherein the crosslinked polyamine particles comprise polyallylamine crosslinked with from 7-12 wt. % epichlorohydrin, the crosslinked polyallylamine particles being formed by aggregating 2 or more constituent particles comprising polyallylamine crosslinked with 7-12 wt. % epichlorohydrin.

In some embodiments, the invention is, consists essentially of, or comprises crosslinked polyamine particles, a pharmaceutical composition comprising crosslinked polyamine particles or a method for removing a compound or ion, such as a phosphorous-containing compound or a phosphorous-containing ion (e.g., phosphate), from the gastrointestinal tract of an animal by administering an effective amount of crosslinked polyamine particles or a pharmaceutical composition comprising crosslinked polyamine particles, wherein the crosslinked polyamine particles comprise polyallylamine crosslinked with from 7-12 wt. % epichlorohydrin, the crosslinked polyallylamine particles having an in vitro competitive phosphate binding capacity of greater than 1.2 mmol/g at 60 minutes.

In some embodiments, crosslinked polyamine particles according to the invention may have one or more of or any combination of the following characteristics:
a) a particle size distribution such that 75 vol. % or greater of the crosslinked polyamine particles have a size of between 250 μm and 4 mm;
b) a particle size distribution where from 5 vol. % to 100 vol. % of the crosslinked polyamine particles have a particle size of greater than 500 μm;
c) a particle size distribution such that no more than 20 vol. % of the crosslinked polyamine particles have a particle size less than 300 μm;
d) a particle size distribution such that the crosslinked polyamine particles have a d₁₀ value that is between 250 μm and 750 μm
e) a particle size distribution such that the crosslinked polyamine particles have a d₉₀ value that is between 900 μm and 1600 μm;
f) a particle size distribution such that the crosslinked polyamine particles have a d₅₀ between 450 μm and 1100 μm;
g) from 75 wt. % to 100 wt. % of the crosslinked polyamine particles have a mesh size that is −5/+60;
h) from 5 wt. % to 100 wt. % of the crosslinked polyamine particles have a mesh size that is +35;
i) no more than 20 wt. % of the crosslinked polyamine particles have a mesh size that is −50;
j) from 40 wt. % to 60 wt. % of the crosslinked polyamine particles have a mesh size that is −16/+40;
k) a mean gray value greater than 180;
l) comprises 2 or more constituent particles; and/or
m) a competitive phosphate binding capacity at 60 minutes of greater than 1.2.

In some embodiments, the crosslinked polyamine particles described herein may comprise aggregates of constituent particles of the crosslinked polyamine polymers. In some embodiments, the constituent particles may have a particle size distribution such that greater than 70% of the constituent particles have a particle size between 50 μm and 850 μm. In some embodiments, the constituent particles may have a particle size distribution such that the constituent particles have a d₁₀ value between about 20 μm and about 100 μm and/or a d₉₀ value that is between about 150 μm and about 450 μm. In some embodiments, the constituent particles may have a d₅₀ between 50 μm and 200 μm. In some embodiments, the crosslinked polyamine particles comprise aggregates of from about 2 to about 10,000 constituent particles.

In some embodiments, the invention provides methods of treating an animal, including a human. The method generally involves administering an effective amount of crosslinked polyamine particles or a composition (e.g., a pharmaceutical composition) comprising the same to the animal as described herein.

In some embodiments, the crosslinked polyamine particles have an in vitro competitive phosphate binding capacity of greater than 0.4 mmol/g throughout a physiologically significant. time period. In some embodiments, the crosslinked polyamine particles have an in vitro competitive phosphate binding capacity of greater than 0.5 mmol/g at 60 minutes. In some embodiments, the crosslinked polyamine particles have an in vitro competitive phosphate binding capacity of less than 1.4 mmol/g at 20 minutes. In some embodiments, the crosslinked polyamine particles have an in vitro competitive binding capacity at 60 minutes that is greater than 20% of the in vitro non-competitive phosphate binding capacity of said polymer at 300 minutes.

In some embodiments, the crosslinked polyamine particles are acid stable or exhibit enhanced acid stability. In some embodiments, the acid stability of the crosslinked polyamine particles is enhanced by curing the particles by exposing the particles to an elevated temperature. In some embodiments, the acid stability of the crosslinked polyamine particles may be improved by curing the crosslinked polyamine particles, such as by holding the crosslinked polyamine particles at an elevated temperature for an extended period of time. In some embodiments, the acid stability may be demonstrated by or may comprise a particle size for acid treated crosslinked polyamine particles that have been cured that is greater than 1.2 fold the particle size of acid treated crosslinked polyamine particles that have not been cured. In some embodiments, the acid stability of the crosslinked polyamine particles may be demonstrated by or may comprise greater than 60% retention of competitive phosphate binding of acid treated particles relative to non-acid treated particles.

Another aspect of the invention is a pharmaceutical composition comprising crosslinked polyamine particles of the present invention and at least one pharmaceutically acceptable excipient. In some embodiments, the composition is a liquid formulation in which the crosslinked polyamine particles are dispersed in a liquid vehicle, such as water, and suitable excipients. In some embodiments, the invention provides a pharmaceutical composition comprising crosslinked polyamine particles for binding a target compound or ion, and one or more suitable pharmaceutical excipients, where the composition is in the form of a tablet, sachet, slurry, food formulation, troche, capsule, elixir, suspension, syrup, wafer, chewing gum or lozenge. In some embodiments the composition contains a pharmaceutical excipient selected from the group consisting of sucrose, mannitol, xylitol, maltodextrin, fructose, sorbitol, and combinations thereof. In some embodiments the target anion of the crosslinked polyamine particles is an organophosphate and/or phosphate. In some embodiments the crosslinked polyamine particles are more than about 50% of the weight of the tablet. In some embodiments, the tablet is of cylindrical shape with a diameter of from about 12 mm to about 28 mm and a height of from about 1 mm to about 8 mm and the crosslinked polyamine particles comprise more than 0.6 to about 2.0 μm of the total weight of the tablet.

In some of the compositions of the invention, the excipients are chosen from the group consisting of sweetening agents, binders, lubricants, and disintegrants. In some of these embodiments, the sweetening agent is selected from the group consisting of sucrose, mannitol, xylitol, maltodextrin, fructose, and sorbitol, and combinations thereof

The crosslinked polyamine particles described herein have several therapeutic applications. For example, the crosslinked polyamine particles are useful in removing compounds or ions such as anions, for example phosphorous-containing compounds or phosphorous containing ions such as organophosphates and/or phosphates, from the gastrointestinal tract, such as from the stomach, small intestine and/or large intestine. In some embodiments, the crosslinked amine polymers are used in the treatment of phosphate imbalance disorders and renal diseases.

In yet another aspect, the crosslinked polyamine particles are useful for removing other solutes, such as chloride, bicarbonate, and/or oxalate containing compounds or ions. Crosslinked polyamine particles removing oxalate compounds or ions find use in the treatment of oxalate imbalance disorders. Crosslinked polyamine particles removing chloride compounds or ions find use in treating acidosis, for example. In some embodiments, the crosslinked polyamine particles are useful for removing fatty acids, bile acids, citrate and related compounds.

Another aspect of the invention is a tablet comprising polyallylamine crosslinked polyallylamine particles with 8-11 wt. % epichlorohydrin, or a pharmaceutically acceptable salt thereof that may be useful for one or more of the uses presented herein. Upon dissolution of the tablet in a solvent (e.g., a phosphate buffer or hydrochloric acid), the resulting crosslinked polyallylamine particles may have a particle size distribution wherein the volume weighted mean is greater than 300 μm or wherein the volume % mode is greater than 300 μm.

The invention further provides compositions containing any of the above crosslinked polyamine particles where the crosslinked polyamine particles are encased in one or more shells.

### BRIEF DESCRIPTION OF THE DRAWINGS

FIGS. 1(a) and 1(b) illustrate the resulting particle distribution by volume percent after dissolution in a phosphate buffer;

FIGS. 1(c) and 1(d) illustrate the resulting particle distribution by volume percent after dissolution in a phosphate buffer;

FIGS. 1(e) and 1(f) illustrate the resulting particle distribution by volume percent after dissolution in a phosphate buffer;

FIGS. 1(g) and 1(h) illustrate the resulting particle distribution by volume percent after dissolution in a phosphate buffer; and

FIGS. 1(i) and 1(j) illustrate the resulting particle distribution by volume percent after dissolution in a phosphate buffer.

### DETAILED DESCRIPTION OF THE INVENTION

In one aspect, the present invention provides crosslinked polyamine particles, compositions and methods of using crosslinked polyamine particles, where the crosslinked polyamine is represented by repeat units according to any of Formulas I-II. In addition, some embodiments may include multiple different repeat units or residues thereof that repeat in a copolymer or polymer. Such polymers may include one or more additional compounds that may be included in a polymer backbone or as pendant groups either individually or as repeating groups.

As used herein, unless otherwise stated, the term “derived from” is understood to mean: produced or obtained from another substance by chemical reaction, especially directly derived from the reactants, for example a crosslinked polyamine may be derived from the reaction of an amine monomer or amine polymer and a linking agent, such as a crosslinking agent resulting in a crosslinked polyamine that is derived from the amine monomer or amine polymer and the crosslinking agent.

In some embodiments, it has been found that the size and/or size distribution of the crosslinked polyamine particles of the invention affect the ion binding, such as the phosphate binding properties of the polymers. In some embodiments, crosslinked polyamine particles of the invention may exhibit enhanced phosphate binding in the presence of competing organic ions throughout a physiologically significant time period while having similar equilibrium phosphate binding properties when compared to smaller particles of the same polymer.

The particle size of the crosslinked polyamine particles may be determined according to the procedure detailed in the Test Procedures. In some embodiments, crosslinked polyamine particles have a particle size distribution such that 75 vol. % or greater, such as 80 vol. % or greater, 85 vol. % or greater, 90 vol. % or greater, 95 vol. % or greater, 99 vol. % or greater, or 100 vol. % of the crosslinked polyamine particles have a particle size between 250 μm and 4 mm, such as between 275 μm and 3.5 mm, between 300 μm and 3.0 mm, between 300 μm and 2.5 mm, between 300 μm and 2.0 mm, between 325 μm and 2.5 mm, between 350 μm and 2.0 mm, between 375 μm and 1.75 mm, between 400 μm and 1500 μm, between 425 μm and 1400 μm, between 450 μm and 1300 μm, between 475 μm and 1200 μm, between 500 μm and 1100 μm, or between 525 μm and 1075 μm.

In some embodiments of the invention, the crosslinked polyamine particles have a particle size distribution such that greater than 5 vol. %, greater than 10 vol. %, greater than 20 vol. %, greater than 30 vol. %, greater than 40 vol. %, greater than 50 vol. %, greater than 60 vol. %, greater than 70 vol. %, greater than 80 vol. %, greater than 90 vol. % or greater than 95 vol. % of the crosslinked polyamine particles have a particle size of greater than 450 μm, such as greater than 500 μm, greater than 525 μm, greater than 550 μm, greater than 575 μm, greater than 600 μm, greater than 625 μm, greater than 650 μm, greater than 675, greater than 700 μm, greater than 725 μm, greater than 750 μm or greater than 775 μm.

In some embodiments of the invention, the crosslinked polyamine particles have a particle size distribution such that greater than 5 vol. %, greater than 10 vol. %, greater than 20 vol. %, greater than 30 vol. %, greater than 40 vol. %, greater than 50 vol. %, greater than 60 vol. %, greater than 70 vol. %, greater than 80 vol. %, greater than 90 vol. % or greater than 95 vol. % of the crosslinked polyamine particles have a particle size of between 500 μm and 2.0 mm, such as between 525 μm and 1800 μm, between 550 μm and 1600 μm, between 575 μm and 1550 μm, between 600 μm and 1500 μm, between 625 μm and 1475 μm, between 650 μm and 1450 μm, between 675 μm and 1425 μm, between 700 μm and 1400 μm, between 725 μm and 1375 between 750 μm and 1350 μm or between 775 μm and 1300 μm.

In some embodiments of the invention, the crosslinked polyamine particles have a particle size distribution such that from 5 to 100 vol. %, 10 to 90 vol. %, 20 to 80 vol. %, 30 to 70 vol. %, 40 to 60 vol. % or 50 vol. % of the crosslinked polyamine particles have a particle size of greater than 450 μm, such as greater than 500 μm, greater than 525 μm, greater than 550 μm, greater than 575 μm, greater than 600 μm, greater than 625 μm, greater than 650 μm, greater than 675 μm, greater than 700 μm, greater than 725 μm, greater than 750 μm or greater than 775 μm.

In some embodiments of the invention, the crosslinked polyamine particles have a particle size distribution such that from 5 to 100 vol. %, 10 to 90 vol. %, 20 to 80 vol. %, 30 to 70 vol. %, 40 to 60 vol. % or 50 vol. % of the crosslinked polyamine particles have a particle size of between 500 μm and 2.0 mm, such as between 525 μm and 1800 μm, between 550 μm and 1600 μm, between 575 μm and 1550 μm, between 600 μm and 1500 μm, between 625 μm and 1475 μm, between 650 μm and 1450 μm, between 675 μm and 1425 μm, between 700 μm and 1400 μm, between 725 μm and 1375 μm, between 750 μm and 1350 μm or between 775 μm and 1300 μm.

In some embodiments of the invention, the crosslinked polyamine particles have a particle size distribution such that no more than 0 to 20 vol. %, such as no more than 5 to 15 vol. %, such as no more than 5 vol. %, 10 vol. %, 15 vol. % or 20 vol. % of the crosslinked polyamine particles have a particle size of less than about 300 μm. In some embodiments of the invention, the crosslinked polyamine particles have a particle size distribution such that no more than 0 to 25 vol. %, such as no more than 5 to 20 vol. %, such as no more than 5 vol. %, 10 vol. %, 15 vol. %, 20 vol. % or no more than 25 vol. % of the crosslinked polyamine particles have a particle size of less than about 350 μm. In some embodiments of the invention, the crosslinked polyamine particles have a particle size distribution such that no more than 0 to 35 vol. %, such as no more than 5 to 30 vol. %, such as no more than 10 vol. %, 15 vol. %, 20 vol. %, 25 vol. % or no more than 30 vol. % of the crosslinked polyamine particles have a particle size of less than about 400 μm. In some embodiments of the invention, the crosslinked polyamine particles have a particle size distribution such that no more than 0 to 40 vol. %, such as no more than 5 to 35 vol. %, such as no more than 10 vol. %, 15 vol. %, 20 vol. %, 25 vol. %, 20 vol. %, 35 vol. % or no more than 40 vol. % of the crosslinked polyamine particles has a particle size of less than about 450 μm.

In some embodiments of the invention, the crosslinked polyamine particles have a particle size distribution such that d₁₀ is greater than 225 μm, such as greater than 250 μm, greater than 275 μm, greater than 300 μm, greater than 325 μm, greater than 350 μm, greater than 375 μm, greater than 400 μm, greater than 425, μm, greater than 450 μm, greater than 475 μm, greater than 500 μm, greater than 525 μm, or greater than 550 μm.

In some embodiments of the invention, the crosslinked polyamine particles have a particle size distribution such that d₁₀ is between 275 μm and 725 μm, between 300 μm and 700 μm, between 325 μm and 675 μm, between 350 μm and 650 μm, between 375 μm and 625 μm.

In some embodiments of the invention, the crosslinked polyamine particles have a particle size distribution such that d₉₀ is less than 1650 μm, such as less than 1600 μm, less than 1550 μm, less than 1500 μm, less than 1475 μm, less than 1450 μm, less than 1425 μm, less than 1400 μm, less than 1350 μm, less than 1300 μm.

In some embodiments of the invention, the crosslinked polyamine particles have a particle size distribution such that d₉₀ is between 900 μm and 1600 μm, such as between 925 μm and 1550 μm, between 950 μm and 1525 μm, between 975 μm and 1500 μm, between 1000 μm and 1475 μm, between 1025 μm and 1450 μm, between 1050 μm and 1425 μm, between 1075 μm and 1400 μm, between 1100 μm and 1400 μm, between 1100 μm and 1375 μm, between 1100 μm and 1350 μm or between 1100 μm and 1325 μm.

In some embodiments of the invention, the crosslinked polyamine particles have a particle size distribution such that d₁₀ is greater than 225 μm, such as greater than 250 μm, greater than 275 μm, greater than 300 μm, greater than 325 μm, greater than 350 μm, greater than 375 μm, greater than 400 μm, greater than 425 μm, greater than 450 μm, greater than 475 μm, greater than 500 μm, greater than 525 μm, or greater than 550 μm and d₉₀ is less than 1650 μm, such as less than 1600 μm, less than 1550 μm, less than 1500 μm, less than 1475 μm, less than 1450 μm, less than 1425 μm, less than 1400 μm, less than 1350 μm, less than 1300 μm.

In some embodiments of the invention, the crosslinked polyamine particles have a particle size distribution such that d₁₀ is greater than 225 μm, such as greater than 250 μm, greater than 275 μm, greater than 300 μm, greater than 325 μm, greater than 350 μm, greater than 375 μm, greater than 400 μm, greater than 425, μm, greater than 450 μm, greater than 475 μm, greater than 500 μm, greater than 525 μm, or greater than 550 μm and d₉₀ is between 900 μm and 1600 μm, such as between 925 μm and 1550 μm, between 950 μm and 1525 μm, between 975 μm and 1500 μm, between 1000 μm and 1475 μm, between 1025 μm and 1450 μm, between 1050 μm and 1425 μm, between 1075 μm and 1400 μm, between 1100 μm and 1400 μm, between 1100 μm and 1375 m, between 1100 μm and 1350 μm or between 1100 μm and 1325 μm.

In some embodiments of the invention, the crosslinked polyamine particles have a particle size distribution such that d₁₀ is between 275 μm and 725 μm, between 300 μm and 700 μm, between 325 μm and 675 μm, between 350 μm and 650 μm, between 375 μm and 625 μm and d₉₀ is less than 1650 μm, such as less than 1600 μm, less than 1550 μm, less than 1500 μm, less than 1475 μm, less than 1450 μm, less than 1425 μm, less than 1400 μm, less than 1350 μm, less than 1300 μm.

In some embodiments of the invention, the crosslinked polyamine particles have a particle size distribution such that d₁₀ is between 275 μm and 725 μm, between 300 μm and 700 μm, between 325 μm and 675 μm, between 350 μm and 650 μm, between 375 μm and 625 μm and d₉₀ is between 900 μm and 1600 μm, such as between 925 μm and 1550 μm, between 950 μm and 1525 μm, between 975 μm and 1500 μm, between 1000 μm and 1475 μm, between 1025 μm and 1450 μm, between 1050 μm and 1425 μm, between 1075 μm and 1400 μm, between 1100 μm and 1400 μm, between 1100 μm and 1375 μm, between 1100 μm and 1350 μm or between 1100 μm and 1325 μm.

In some embodiments of the invention, the crosslinked polyamine particles have a d₅₀ that is greater than 450 μm, such as greater than 475 μm, greater than 500 μm, greater than 525 μm, greater than 550 μm, greater than 575 μm, greater than 600 μm, greater than 625 tim, greater than 650 μm, greater than 675 μm. In or greater than 700 μm.

In some embodiments of the invention, the crosslinked polyamine particles have a d₅₀ between 450 μm and 1100 μm, such as between 475 μm and 1050 μm, between 500 μm and 1025 μm, between 525 μm and 1000 μm, between 550 μm and 975 μm, between 575 μm and 950 μm, between 600 μm and 925 μm, between 625 μm and 900 μm, between 650 μm and 875 μm, between 675 μm and 850 μm or between 700 μm and 825 μm. In some embodiments, the crosslinked polyamine particles have a d₅₀ between 675 μm and 1000 μm.

In some embodiments, crosslinked polyamine particles of the invention may be sized according to sieve size with a “+” indicating that the crosslinked polyamine particles are held back by a sieve of the indicated mesh size and a “−” indicating that the crosslinked polyamine particles pass through a sieve of the indicated mesh size. Thus a crosslinked polyamine particle that passes through a No. 5 mesh sieve but is held back by a No. 20 mesh sieve is designated as being −5/+20. All references to mesh size described herein refer to mesh sizes that are U.S. Standard and in conformance with ASTM E-11. In some embodiments, from 75 wt. % to 100 wt. %, such as 80 wt. %, 85 wt. %, 90 wt. % or 95 wt. % of the crosslinked polyamine particles have a mesh size that is −5, −6, −7, −8, −10, 12, −14, −16, −18, −20, or −25. In some embodiments from 50 to 100 wt. % such as 55 wt. %, 60 wt. %, 65 wt. %, 70 wt. %, 75 wt. %, 80 wt. %, 85 wt. %, 90 wt. % or 95 wt. % of the crosslinked polyamine particles have a mesh size that is +60, +50, +45, +40, +35 or +30. In some embodiments, from 50 wt. % to 100 wt. % such as 55 wt. %, 60 wt. %, 65 wt. %, 70 wt. %, 75 wt. %, 80 wt. %, 85 wt. %, 90 wt. % or 95 wt. % of the crosslinked polyamine particles have a mesh size that is −5/+60, such as −6/+60, −7/+60, −8/+60, −10/+60, −12/+60, −14/+60, −16/+50, −18/+50, −20/+50, −25/+45, −25/+40, −25/+35 or −25/+30. In some embodiments, from 40 wt. % to 60 wt. % of the crosslinked polyamine particles have a mesh size that is −16/+40 mesh, such as −18/+35, −20/+35, −20/+30 or −20/+25.

In some embodiments of the invention, from 5 to 100 wt. % of the crosslinked polyamine particles, such as 10 to 90 wt. %, 20 to 80 wt. %, 30 to 70 wt. %, 40 to 60 wt. % or 50 wt. % of the crosslinked polyamine particles have a mesh size that +35 mesh, such as +30, +25, +20, +18, +16, or +14 mesh.

In some embodiments of the invention, greater than 10 wt. %, greater than 20 wt. %, greater than 30 wt. %, greater than 40 wt. %, greater than 50 wt. %, greater than 60 wt. %, greater than 70 wt. %, greater than 80 wt. %, greater than 90 wt. % or greater than 95 wt. % of the crosslinked polyamine particles have a mesh size of +35 mesh, such as +30, +25, +20, +18, +16, or +14 mesh.

In some embodiments of the invention, no more than 0 to 20 wt. %, such as no more than 5 to 15 wt. %, such as no more than 10 wt. % of the crosslinked polyamine particles have a mesh size that is −50. In some embodiments of the invention, no more than 0 to 25 wt. %, such as no more than 5 to 20 wt. %, such as no more than 10 wt. % or no more than 15 wt. % of the crosslinked polyamine particles have a mesh size that is −45. In some embodiments of the invention, no more than 0 to 35 wt. %, such as no more than 5 to 35 wt. %, such as no more than 10 wt. %, 15 wt. %, 20 wt. %, 25 wt. % or no more than 20 wt. % of the crosslinked polyamine particles have a mesh size that is −40. In some embodiments of the invention, no more than 0 to 45 wt. %, such as no more than 5 to 30 wt. %, such as no more than 10 wt. %, 15 wt. %, 20 wt. %, 25 wt. %, 30 wt. %, 35 wt. %, or no more than 40 wt. % have a mesh size that is −35.

In some embodiments, crosslinked polyamine particles of the invention may have any one or more of the particle size characteristics described herein prior to being formulated into a final dosage form, while in other embodiments, crosslinked polyamine particles of the invention may have any one or more of the particle size characteristics described herein when in a final dosage form. In some embodiments, any of the particle size characteristics described above may be determined prior to tableting. In other embodiments, any of the particle size characteristics described above may be determined after tableting has occurred.

Any suitable method of controlling or achieving the desired particle size may be used. For example, the particle size of the crosslinked polyamine particles may be controlled by controlling various polymerization process parameters such as temperature, monomer and crosslinker concentration, solvent, monomer to solvent ratio, pH, infusion rate, mixing rate, and by selecting the downstream process and processing parameters. For example, the particle size may be affected by the orifice size of a spray dryer nozzle and the height of a spray drying tower or the drying temperature. In addition, after polymerization, the crosslinked polyamine particles may be further processed to achieve the desired particle size such as ground using a grinder or a mill or selectively sieved. Any suitable method of controlling or achieving the desired particle size may be used. Specific suitable downstream processing methods include, but are not limited to grinding, wet or dry milling, spray drying, sieving, precipitation, and spray-freezing. In some embodiments, the down stream processing methods comprise wet milling.

In some embodiments, it has been found that the size and/or size distribution of the crosslinked polyamine particles of the invention affect the ion binding, such as the phosphate binding properties of the polymers. In some embodiments, crosslinked polyamine particles of the invention may exhibit enhanced phosphate binding in the presence of competing organic ions throughout a physiologically significant time period while having similar equilibrium phosphate binding properties when compared to smaller particles of the same polymer.

Accordingly, in some embodiments, the crosslinked polyamine particles may have one or more of the following particle size characteristics, such as 1, 2, 3, 4, 5, 6, 7, 8, 9 or even all 10 of the following particle size characteristics as discussed above:
a) a particle size distribution such that 75 vol. % or greater of the crosslinked polyallylamine particles have a size of between 250 μm and 4 mm;
b) a particle size distribution where from 5 vol. % to 100 vol. % of the crosslinked polyallylamine particles have a particle size of greater than 500 μm;
c) a particle size distribution such that no more than 20 vol. % of the crosslinked polyallylamine particles have a particle size less than 300 μm;
d) a particle size distribution such that the crosslinked polyamine particles have a d₁₀ value that is between 250 μm and 750 μm
e) a particle size distribution such that the crosslinked polyamine particles have a d₉₀ value that is between 900 μm and 1600 μm;
f) a particle size distribution such that the crosslinked polyamine particles have a d₅₀ between 450 μm and 1100 μm;
g) from 75 wt. % to 100 wt. % of the crosslinked polyamine particles have a mesh size that is −5/+60;
h) from 5 wt. % to 100 wt. % of the crosslinked polyamine particles have a mesh size that is +35;
i) no more than 20 wt. % of the crosslinked polyamine particles have a mesh size that is −50; and/or
j) from 40 wt. % to 60 wt. % of the crosslinked polyamine particles have a mesh size that is −16/+40.

Thus, by way of example, in some embodiments, the crosslinked polyallylarnines may have 3 of the above particle size characteristics such as a, e and h (or aeh) and would thus have a particle size distribution such that 75 vol. % or greater of the crosslinked polyallylamine particles have a size of between 250 μm and 4 mm, a particle size distribution such that the crosslinked polyamine particles have a d₉₀ value that is between 900 μm and 1600 μm and from 5 wt. % to 100 wt. % of the crosslinked polyamine particles have a mesh size that is +35. Accordingly it should be understood that the crosslinked polyamine particles may have any one or more of the above characteristics in any combination. Similarly, when any characteristics herein are provided in a list that includes “and/or” it should be understood that each and every possible permutation of combinations of those characteristics are specifically disclosed and included herein.

In addition, it should be understood that each of the characteristics identified herein by a letter such as “a)” may be any permutation of that same characteristic as discussed in the various detail paragraphs herein. For example, characteristic “a)” refers to a particle size distribution such that 75 vol. % or greater of the crosslinked polyallylamine particles have a size of between 250 μm and 4 mm. This reference however should be understood to encompass the detailed discussion of this characteristic above where it is shown that characteristic “a)” refers to particles having a particle size distribution such that 75 vol. % or greater, such as 80 vol. % or greater, 85 vol. % or greater, 90 vol. % or greater, 95 vol. % or greater, 99 vol. % or greater, or 100 vol. % of the crosslinked polyamine particles have a particle size between 250 μm and 4 mm, such as between 275 μm and 3.5 mm, between 300 μm and 3.0 mm, between 300 μm and 2.5 mm, between 300 μm and 2.0 mm, between 325 μm and 2.5 mm, between 350 μm and 2.0 mm, between 375 μm and 1.75 mm, between 400 μm and 1500 μm, between 425 μm and 1400 μm, between 450 μm and 1300 μm, between 475 μm and 1200 μm, between 500 μm and 1100 μm, or between 525 μm and 1075 μm. Each of the individual characteristics identified by letters in this application should be understood to refer to their detail paragraph or paragraphs discussed elsewhere in this application.

In some embodiments, crosslinked polyamine particles according to the invention exhibit special optical characteristics, such as optical density. In some embodiments, the crosslinked polyamine particles may have a mean gray value of greater than 180, such as a mean gray value of greater than 185, greater than 190, greater than 195, greater than 200, greater than 205, greater than 210, greater than 215 or greater than 220. In some embodiments, crosslinked polyamine particles according to the invention have a mean gray value that is between 180 and 230, such as between 185 and 225, between 190 and 215, between 190 and 210, between 195 and 205 or between 195 and 200. The mean gray value may be measured according to the techniques described in the Test Methods section below.

In some embodiments, the crosslinked polyamine particles described herein may comprise constituent particles or may comprise aggregates of constituent particles of the crosslinked polyamine polymers. In some embodiments, the constituent particles may have a particle size distribution such that greater than 70%, such as greater than 80 vol. %, such as greater than 85 vol. %, greater than 90 vol. %, greater than 95 vol. %, greater than 99 vol. % or 100 vol. % of the constituent particles have particle size between 10 μm and 850 μm, such as between 10 μm and 800 μm, between 10 μm and 750 μm, between 10 μm and 650 μm, between 10 μm and 550 μm, between 10μm and 450 μm, between 10 μm and 400 μm, between 20 μm and 650 μm, between 30 μm and 550 μm, between 40 μm and 450 μm, between 50 μm and 400 μm, between 55 μm and 750 μm, between 55 μm and 650 μm, between 55 μm and 550 μm, between 55 μm and 500 μm, between 55 μm and 450 μm, between 55 μm and 400 μm, between 60 μm and 350 μm, between 65 μm and 300 μm, between 70 μm and 250 μm, between 75 μm and 200 μm, between 85 μm and 150 μm, between 90 μm and 125 μm or between 90 μm and 105 μm.

In some embodiments, the crosslinked polyamine particles described herein may comprise constituent particles or may comprise aggregates of constituent particles where the constituent particles have a particle size distribution such that the constituent particles have a d₁₀ value between 20 μm and 100 μm, such as between 20 μm and 70 μm, between 25 μm and 60 μm, between 28 μm and 53 μm, or between 30 μm and 50 m.

In some embodiments, the crosslinked polyamine particles described herein may comprise constituent particles or may comprise aggregates of constituent particles where the constituent particles have a particle size distribution such that the constituent particles have a d₁₀ value greater than 20 μm, greater than 25 μm, greater than 28 μm or greater than 30 μm.

In some embodiments, the crosslinked polyamine particles described herein may comprise constituent particles or may comprise aggregates of constituent particles where the constituent particles have a particle size distribution such that the constituent particles have a d₉₀ value that is between 120 μm and 450 μm, such as between 150 μm and 400 μm, between 175 μm and 350 μm, between 175 μm and 300 μm, between 175 μm and 275 μm or between 175 μm and 250 μm.

In some embodiments, the crosslinked polyamine particles described herein may comprise constituent particles or may comprise aggregates of constituent particles where the constituent particles have a particle size distribution such that the constituent particles have a d₉₀ value that is less than 450 μm, such as less than 425 μm, less than 400 μm, less than 375 μm, less than 350 μm, less than 325 μm, less than 300 μm, less than 275 μm or less than 250 μm.

In some embodiments, the crosslinked polyamine particles described herein may comprise constituent particles or may comprise aggregates of constituent particles where the constituent particles have a particle size distribution such that the constituent particles have a d₁₀ value between 20 μm and 100 μm, such as between 20 μm and 70 μm, between 25 μm and 60 μm, between 28 μm and 53 μm, or between 30 μm and 50 μm and a d₉₀ value that is between 120 μm and 450 μm, such as between 150 μm and 400 μm, between 175 μm and 350 μm, between 175 μm and 300 μm, between 175 μm and 275 μm or between 175 μm and 250 μm.

In some embodiments, the crosslinked polyamine particles described herein may comprise constituent particles or may comprise aggregates of constituent particles where the constituent particles have a particle size distribution such that the constituent particles have a d10 value between 20 μm and 100 μm, such as between 20 μm and 70 μm, between 25 μm and 60 μm, between 28 μm and 53 μm, or between 30 μm and 50 μm and a d₉₀ value that is less than 450 such as less than 425 μm, less than 400 μm, less than 375 μm, less than 350 μm, less than 325 μm, less than 300 μm, less than 275 μm or less than 250 μm.

In some embodiments, the crosslinked polyamine particles described herein may comprise constituent particles or may comprise aggregates of constituent particles where the constituent particles have a particle size distribution such that the constituent particles have a d₁₀ value greater than 20 μm, greater than 25 μm, greater than 28 μm or greater than 30 μm and a d₉₀ value that is between 120 μm and 450 μm, such as between 150 μm and 400 μm, between 175 μm and 350 μm, between 175 μm and 300 μm, between 175 μm and 275 μm or between 175 μm and 250 μm.

In some embodiments, the crosslinked polyamine particles described herein may comprise constituent particles or may comprise aggregates of constituent particles where the constituent particles have a particle size distribution such that the constituent particles have a d₁₀ value greater than 20 μm, greater than 25 μm, greater than 28 μm or greater than 30 μm and a d₉₀ value that is less than 450 μm, such as less than 425 μm, less than 400 μm, less than 375 μm, less than 350 μm, less than 325 μm, less than 300 μm, less than 275 μm or less than 250 μm.

In some embodiments, the crosslinked polyamine particles described herein may comprise constituent particles or may comprise aggregates of constituent particles where the constituent particles have a d₅₀ between 50 μm and 200 μm, such as between 50 μm and 175 μm, between 50 μm and 150 μm, between 50 μm and 120 μm, between 70 μm and 120 μm or between 70 μm and 100 μm.

In some embodiments, the crosslinked polyamine particles comprise 2 or more constituent particles, such as from 2 to 10,000 constituent particles, such as from 10 to 9000 constituent particles, from 100 to 8000 constituent particles, from 150 to 7000 constituent particles, from 200 to 6000 constituent particles, from 250 to 5000 constituent particles, from 275 to 4000 constituent particles, from 300 to 3500 constituent particles, from 350 to 3000 constituent particles, from 400 to 2500 constituent particles, from 450 to 2000 constituent particles, from 500 to 1500 constituent particles, from 600 to 1250 constituent particles, from 700 to 1000 constituent particles. In some embodiments, the crosslinked polyamine particles comprise from 500 to 1000 constituent particles.

In some embodiments, the crosslinked polyamine particles comprise aggregates of 2 or more constituent particles, such as from 2 to 10,000 constituent particles, such as from 10 to 9000 constituent particles, from 100 to 8000 constituent particles, from 150 to 7000 constituent particles, from 200 to 6000 constituent particles, from 250 to 5000 constituent particles, from 275 to 4000 constituent particles, from 300 to 3500 constituent particles, from 350 to 3000 constituent particles, from 400 to 2500 constituent particles, from 450 to 2000 constituent particles, from 500 to 1500 constituent particles, from 600 to 1250 constituent particles, from 700 to 1000 constituent particles. In some embodiments, the crosslinked polyamine particles comprise aggregates of from 500 to 1000 constituent particles.

In some embodiments, the crosslinked polyamine particles described herein may comprise particles which are formed by aggregating 2 or more constituent particles. In some embodiments, the constituent particles may have a particle size distribution such that greater than 70%, such as greater than 80 vol. %, such as greater than 85 vol. %, greater than 90 vol. %, greater than 95 vol. %, greater than 99 vol. % or 100 vol. % of the constituent particles have particle size between 10 μm and 850 μm, such as between 10 μm and 800 μm, between 10 μm and 750 μm, between 10 μm and 650 μm, between 10 μm and 550 μm, between 10 μm and 450 μm, between 10 μm and 400 μm, between 20 μm and 650 μm, between 30 μm and 550 μm, between 40 μm and 450 μm, between 50 μm and 400 μm, between 55 μm and 750 μm, between 55 μm and 650 μm, between 55 μm and 550 μm, between 55 μm and 500 μm, between 55 μm and 450 μm, between 55 μm and 400 μm, between 60 μm and 350 μm, between 65 μm and 300 μm, between 70 μm and 250 μm, between 75 μm and 200 μm, between 85 μm and 150 μm, between 90 μm and 125 μm or between 90 μm and 105 μm.

In some embodiments, the crosslinked polyamine particles described herein may comprise particles which are formed by aggregating 2 or more constituent particles where the constituent particles have a particle size distribution such that the constituent particles have a d₁₀ value between 20 μm and 100 μm, such as between 20 μm and 70 μm, between 25 μm and 60 μm, between 28 μm and 53 μm, or between 30 μm and 50 μm.

In some embodiments, the crosslinked polyamine particles described herein may comprise particles which are formed by aggregating 2 or more constituent particles where the constituent particles have a particle size distribution such that the constituent particles have a dₘ value greater than 20 μm, greater than 25 μm, greater than 28 μm or greater than 30 μm.

In some embodiments, the crosslinked polyamine particles described herein may comprise particles which are formed by aggregating 2 or more constituent particles where the constituent particles have a particle size distribution such that the constituent particles have a d₉₀ value that is between 120 μm and 450 μm, such as between 150 μm and 400 μm, between 175 μm and 350 μm, between 175 μm and 300 μm, between 175 μm and 275 μm or between 175 μm and 250 μm.

In some embodiments, the crosslinked polyamine particles described herein may comprise particles which are formed by aggregating 2 or more constituent particles where the constituent particles have a particle size distribution such that the constituent particles have a d₉₀ value that is less than 450 μm, such as less than 425 μm, less than 400 μm, less than 375 μm, less than 350 μm, less than 325 μm, less than 300 μIn, less than 275 μm or less than 250 μm.

In some embodiments, the crosslinked polyamine particles described herein may comprise particles which are formed by aggregating 2 or more constituent particles where the constituent particles have a particle size distribution such that the constituent particles have a d₁₀ value between 20 μm and 100 μm, such as between 20 μm and 70 μm, between 25 μm and 60 μm, between 28 μm and 53 μm, or between 30 μm and 50 μm and a d₉₀ value that is between 120 μm and 450 μm, such as between 150 μm and 400 μm, between 175 μm and 350 μm, between 175 μm and 300 μm, between 175 μm and 275 μm or between 175 μm and 250 μm.

In some embodiments, the crosslinked polyamine particles described herein may comprise particles which are formed by aggregating 2 or more constituent particles where the constituent particles have a particle size distribution such that the constituent particles have a d₁₀ value between 20 μm and 100 μm, such as between 20 μm and 70 μm, between 25 μm and 60 μm, between 28 μm and 53 μm, or between 30 μm and 50 μm and a d₉₀ value that is less than 450 μm, such as less than 425 μm, less than 400 μm, less than 375 μm, less than 350 μm, less than 325 μm, less than 300 μm, less than 275 μm or less than 250 μm.

In some embodiments, the crosslinked polyamine particles described herein may comprise particles which are formed by aggregating 2 or more constituent particles where the constituent particles have a particle size distribution such that the constituent particles have a d₁₀ value greater than 20 μm, greater than 25 μm, greater than 28 μm or greater than 30 μm and a d₉₀ value that is between 120 μm and 450 μm, such as between 150 μm and 400 μm, between 175 μm and 350 μm, between 175 μm and 300 μm, between 175 μm and 275 μm or between 175 μm and 250 μm.

In some embodiments, the crosslinked polyamine particles described herein may comprise particles which are formed by aggregating 2 or more constituent particles where the constituent particles have a particle size distribution such that the constituent particles have a d₁₀ value greater than 20 μm, greater than 25 μm, greater than 28 μm or greater than 30 μm and a d₉₀ value that is less than 450 μm, such as less than 425 μm, less than 400 μm, less than 375 μm, less than 350 μm, less than 325 μm, less than 300 μm, less than 275 μm or less than 250 μm.

In some embodiments, the crosslinked polyamine particles described herein may comprise particles which are formed by aggregating 2 or more constituent particles where the constituent particles have a d₅₀ between 50 μm and 200 μm, such as between 50 μm and 175 μm, between 50 μm and 150 μm, between 50 μm and 120 μm, between 70 μm and 120 μm or between 70 μm and 100 μm.

In some embodiments, the composition may comprise particles which are formed by aggregating 2 or more constituent particles, such as from 2 to 10,000 constituent particles, such as from 10 to 9000 constituent particles, from 100 to 8000 constituent particles, from 150 to 7000 constituent particles, from 200 to 6000 constituent particles, from 250 to 5000 constituent particles, from 275 to 4000 constituent particles, from 300 to 3500 constituent particles, from 350 to 3000 constituent particles, from 400 to 2500 constituent particles, from 450 to 2000 constituent particles, from 500 to 1500 constituent particles, from 600 to 1250 constituent particles, from 700 to 1000 constituent particles. In some embodiments, the crosslinked polyamine particles comprise from 500 to 1000 constituent particles.

In some embodiments, aggregating 2 or more constituent particles includes hydrating constituent particles, such as suspending, forming a suspension of or forming a re-suspension of constituent particles in water. In some embodiments, forming a suspension of or forming a re-suspension of constituent particles includes protonating, such as carbonating, at leaSt a portion of the amines in at least a portion of the crosslinked polyamine particles. In some embodiments, forming includes making a gel from constituent particles. In some embodiments, the gel may be dried and/or the gel may be ground, milled or wet milled.

In some embodiments, the particles may be formed from crosslinked polyamine gel that prior to drying is optionally co-milled, then partially dried (e.g., to a 25-40% LOD), further co-milled and then dried (e.g., to less than 5% LOD), sieved and finally dried.

In some embodiments, the crosslinked polyamine particles according to the invention may have an in vitro competitive phosphate binding capacity at 60 minutes that is greater than 1.2 mmol phosphate/g of polymer, such as greater than 1.25 mmol/g, greater than 1.30 mmol/g, greater than 1.35 mmol/g, greater than 1.4 mmol/g, greater than 1.5 mmol/g, greater than 1.6 mmol/g, greater than 1.7 mmol/g, greater than 1.8 mmol/g, greater than 1.9 mmol/g or greater than 2.0 mmol/g. In some embodiments, the crosslinked polyamine particles according to the invention may have an in vitro competitive phosphate binging capacity at 60 minutes that is between 1.2 mmol/g and 10 mmol/g, such as between 1.2 mmol/g and 7.5 mmol/g, between 1.2 mmol/g and 5.0 mmol/g, between 1.2 mmol/g and 4.0 mmol/g, between 1.25 mmol/g and 4.0 mmol/g, between 1.3 mmol/g and 4.0 mmol/g, between 1.35 mmol/g and 4.0 mmol/g, between 1.4 mmol/g and 4.0 mmol/g, between 1.5 mmol/g and 4.0 mmol/g, between 1.6 mmol/g and 4.0 mmol/g, between 1.7 mmol/g and 4.0 mmol/g, or between 1.8 mmol/g and 4.0 mmol/g.

In some embodiments, the invention is, consists essentially of, or comprises crosslinked polyamine particles, a pharmaceutical composition comprising crosslinked polyamine particles or a method for removing a compound or ion, such as a phosphorous-containing compound or a phosphorous-containing ion (e.g., phosphate), from the gastrointestinal tract of an animal by administering an effective amount of crosslinked polyamine particles or a pharmaceutical composition comprising crosslinked polyamine particles, where the crosslinked polyamine particles comprise polyallylamine crosslinked with 7-12 wt. % epichlorohydrin where the crosslinked polyallylamine particles have one or more of the following characteristics:
a) a particle size distribution such that 75 vol. % or greater of the crosslinked polyallylamine particles have a size of between 250 μm and 4 mm;
b) a particle size distribution where from 5 vol. % to 100 vol. % of the crosslinked polyallylamine particles have a particle size of greater than 500 μm;
c) a particle size distribution such that no more than 20 vol. % of the crosslinked polyallylamine particles have a particle size less than 300 μm;
d) a particle size distribution such that the crosslinked polyamine particles have a d₁₀ value that is between 250 μm and 750 μm
e) a particle size distribution such that the crosslinked polyamine particles have a d₉₀ value that is between 900 μm and 1600 μm;
f) a particle size distribution such that the crosslinked polyamine particles have a d₅₀ between 450 μm and 1100 μm;
g) from 75 wt. % to 100 wt. % of the crosslinked polyamine particles have a mesh size that is −5/+60;
h) from 5 wt. % to 100 wt. % of the crosslinked polyamine particles have a mesh size that is +35;
i) no more than 20 wt. % of the crosslinked polyamine particles have a mesh size that is −50;
j) from 40 wt. % to 60 wt. % of the crosslinked polyamine particles have a mesh size that is −16/+40;
k) a mean gray value greater than 180;
l) comprises 2 or more constituent particles; and/or
m) a competitive phosphate binding capacity at 60 minutes of greater than 1.2.

In some embodiments, the invention is, consists essentially of, or comprises crosslinked polyamine particles, a pharmaceutical composition comprising crosslinked polyamine particles or a method for removing a compound or ion, such as a phosphorous-containing compound or a phosphorous-containing ion (e.g., phosphate), from the gastrointestinal tract of an animal by administering an effective amount of crosslinked polyamine particles or a pharmaceutical composition comprising crosslinked polyamine particles, where the crosslinked polyamine particles comprise polyallylamine crosslinked with 7-12 wt% epichlorohydrin that is partially or fully protonated with a pharmaceutically acceptable counterion as the counterion and where the crosslinked polyallylamine particles have one or more of the following characteristics:
a) a particle size distribution such that 75 vol. % or greater of the crosslinked polyallylamine particles have a size of between 250 μm and 4 mm;
b) a particle size distribution where from 5 vol. % to 100 vol. % of the crosslinked polyallylamine particles have a particle size of greater than 500 μm;
c) a particle size distribution such that no more than 20 vol. % of the crosslinked polyallylamine particles have a particle size less than 300 μm;
d) a particle size distribution such that the crosslinked polyamine particles have a _{d10} value that is between 250 μm and 750 μm
e) a particle size distribution such that the crosslinked polyamine particles have a d₉₀ value that is between 900 μm and 1600 μm;
f) a particle size distribution such that the crosslinked polyamine particles have a d₅₀ between 450 μm and 1100 μm;
g) from 75 wt. % to 100 wt. % of the crosslinked polyamine particles have a mesh size that is −5/+60;
h) from 5 wt. % to ‘100 wt. % of the crosslinked polyamine particles have a mesh size that is +35;
i) no more than 20 wt. % of the crosslinked polyamine particles have a mesh size that is −50;
j) from 40 wt. % to 60 wt. % of the crosslinked polyamine particles have a mesh size that is −16/+40;
k) a mean gray value greater than 180;
l) comprises 2 or more constituent particles; and/or
m) a competitive phosphate binding capacity at 60 minutes of greater than 1.2.

In some embodiments, the invention is, consists essentially of, or comprises crosslinked polyamine particles, a pharmaceutical composition comprising crosslinked polyamine particles or a method for removing a compound or ion, such as a phosphorous-containing compound or a phosphorous-containing ion (e.g., phosphate), from the gastrointestinal tract of an animal by administering an effective amount of crosslinked polyamine particles or a pharmaceutical composition comprising crosslinked polyamine particles, where the crosslinked polyamine particles comprise polyallylamine crosslinked with 7-12wt. % epichlorohydrin that is partially or fully protonated, having carbonate, bicarbonate, hydrochloride or mixtures thereof as the counterion and where the crosslinked polyallylamine particles have one or more of the following characteristics:
a) a particle size distribution such that 75 vol. % or greater of the crosslinked polyallylamine particles have a size of between 250 μm and 4 mm;
b) a particle size distribution where from 5 vol. % to 100 vol. % of the crosslinked polyallylamine particles have a particle size of greater than 500 μm;
c) a particle size distribution such that no more than 20 vol. % of the crosslinked polyallylamine particles have a particle size less than 300 μm;
d) a particle size distribution such that the crosslinked polyamine particles have a _{d10} value that is between 250 μm and 750 μm
e) a particle size distribution such that the crosslinked polyamine particles have a d₉₀ value that is between 900 μm and 1600 μm;
f) a particle size distribution such that the crosslinked polyamine particles have a d₅₀ between 450 μm and 1100 μm;
g) from 75 wt. % to 100 wt. % of the crosslinked polyamine particles have a mesh size that is −5/+60;
h) from 5 wt. % to 100 wt. % of the crosslinked polyamine particles have a mesh size that is +35;
i) no more than 20 wt. % of the crosslinked polyamine particles have a mesh size that is −50;
j) from 40 wt. % to 60 wt. % of the crosslinked polyamine particles have a mesh size that is −16/+40;
k) a mean gray value greater than 180;
l) comprises 2 or more constituent particles; and/or
m) a competitive phosphate binding capacity at 60 minutes of greater than 1.2.

In some embodiments, the invention is, consists essentially of, or comprises crosslinked polyamine particles, a pharmaceutical composition comprising crosslinked polyamine particles or a method for removing a compound or ion, such as a phosphorous-containing compound or a phosphorous-containing ion (e.g., phosphate), from the gastrointestinal tract of an animal by administering an effective amount of crosslinked polyamine particles or a pharmaceutical composition comprising crosslinked polyamine particles, where the crosslinked polyamine particles are derived from: a monomer selected from substituted or unsubstituted allylamine; and a crosslinking agent, where the crosslinked polyamine particles have one or more of the following characteristics:
a) a particle size distribution such that 75 vol. % or greater of the crosslinked polyallylamine particles have a size of between 250 μm and 4 mm;
b) a particle size distribution where from 5 vol. % to 100 vol. % of the crosslinked polyallylamine particles have a particle size of greater than 500 μm;
c) a particle size distribution such that no more than 20 vol. % of the crosslinked polyallylamine particles have a particle size less than 300 μm;
d) a particle size distribution such that the crosslinked polyamine particles have a d₁₀ value that is between 250 μm and 750 μm
e) a particle size distribution such that the crosslinked polyamine particles have a d₉₀ value that is between 900 μm and 1600 μm;
f) a particle size distribution such that the crosslinked polyamine particles have a d₅₀ between 450 μm and 1100 μm;
g) from 75 wt. % to 100 wt. % of the crosslinked polyamine particles have a mesh size that is −5/+60;
h) from 5 wt. % to 100 wt. % of the crosslinked polyamine particles have a mesh size that is +35;
i) no more than 20 wt. % of the crosslinked polyamine particles have a mesh size that is −50;
j) from 40 wt. % to 60 wt. % of the crosslinked polyamine particles have a mesh size that is −16/+40;
k) a mean gray value greater than 180;
l) comprises 2 or more constituent particles; and/or
m) a competitive phosphate binding capacity at 60 minutes of greater than 1.2.

In some embodiments, the invention is, consists essentially of, or comprises crosslinked polyamine particles, a pharmaceutical composition comprising crosslinked polyamine particles or a method for removing a compound or ion, such as a phosphorous-containing compound or a phosphorous-containing ion (e.g., phosphate), from the gastrointestinal tract of an animal by administering an effective amount of crosslinked polyamine particles or a pharmaceutical composition comprising crosslinked polyamine particles, where the crosslinked polyamine particles are derived from: a monomer selected from substituted or unsubstituted allylamine and epichlorohydrin as a crosslinking agent, where the crosslinked polyamine particles have one or more of the following characteristics:
a) a particle size distribution such that 75 vol. % or greater of the crosslinked polyallylamine particles have a size of between 250 μm and 4 mm;
b) a particle size distribution where from 5 vol. % to 100 vol. % of the crosslinked polyallylamine particles have a particle size of greater than 500 μm;
c) a particle size distribution such that no more than 20 vol. % of the crosslinked polyallylamine particles have a particle size less than 300 μm;
d) a particle size distribution such that the crosslinked polyamine particles have a d₁₀ value that is between 250 μm and 750 μm
e) a particle size distribution such that the crosslinked polyamine particles have a d₉₀ value that is between 900 μm and 1600 μm;
f) a particle size distribution such that the crosslinked polyamine particles have a d₅₀ between 450 μm and 1100 μm;
g) from 75 wt. % to 100 wt. % of the crosslinked polyamine particles have a mesh size that is −5/+60;
h) from 5 wt. % to 100 wt. % of the crosslinked polyamine particles have a mesh size that is +35;
i) no more than 20 wt. % of the crosslinked polyamine particles have a mesh size that is −50;
j) from 40 wt. % to 60 wt. % of the crosslinked polyamine particles have a mesh size that is −16/+40;
k) a mean gray value greater than 180;
l) comprises 2 or more constituent particles; and/or
m) a competitive phosphate binding capacity at 60 minutes of greater than 1.2.

In some embodiments, the invention is, consists essentially of, or comprises crosslinked polyamine particles, a pharmaceutical composition comprising crosslinked polyamine particles or a method for removing a compound or ion, such as a phosphorous-containing compound or a phosphorous-containing ion (e.g., phosphate), from the gastrointestinal tract of an animal by administering an effective amount of crosslinked polyamine particles or a pharmaceutical composition comprising crosslinked polyamine particles, where the crosslinked polyamine particles comprise repeat units represented by the following Formula I:

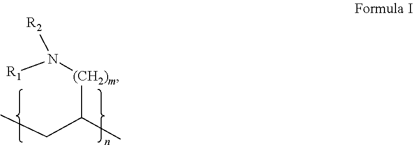

or a copolymer thereof, wherein m is an integer from 0 to 2, such as for example, 0, 1 or 2; n is an integer and each R₁ and each R₂ independently represent hydrogen; substituted or unsubstituted, branched or unbranched C₁-C₆ alkyl, such as C₁, C₂, C₃, C₄, C₅ or C₆ alkyl; or substituted or unsubstituted, branched or unbranched C₁-C₆ alkylamino such as C₁, C₂, C₃, C₄, C₅ or C₆ alkylamino; where the crosslinked polyamine particles have one or more of the following characteristics:
a) a particle size distribution such that 75 vol. % or greater of the crosslinked polyallylamine particles have a size of between 250 μm and 4 mm;
b) a particle size distribution where from 5 vol. % to 100 vol. % of the crosslinked polyallylamine particles have a particle size of greater than 500 μm;
c) a particle size distribution such that no more than 20 vol. % of the crosslinked polyallylamine particles have a particle size less than 300 μm;
d) a particle size distribution such that the crosslinked polyamine particles have a d₁₀ value that is between 250 μm and 750 μm
e) a particle size distribution such that the crosslinked polyamine particles have a d₉₀ value that is between 900 μm and 1600 μm;
f) a particle size distribution such that the crosslinked polyamine particles have a d₅₀ between 450 μm and 1100 μm;
g) from 75 wt. % to 100 wt. % of the crosslinked polyamine particles have a mesh size that is −5/+60;
h) from 5 wt. % to 100 wt. % of the crosslinked polyamine particles have a mesh size that is +35;
i) no more than 20 wt. % of the crosslinked polyamine particles have a mesh size that is −50;
j) from 40 wt. % to 60 wt. % of the crosslinked polyamine particles have a mesh size that is −16/+40;
k) a mean gray value greater than 180;
) comprises 2 or more constituent particles; and/or
m) a competitive phosphate binding capacity at 60 minutes of greater than 1.2.

Examples of some suitable repeat units according to Formula I include:

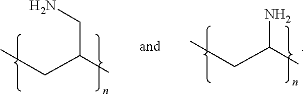

In some embodiments, the invention is, consists essentially of, or comprises crosslinked polyamine particles, a pharmaceutical composition comprising crosslinked polyamine particles or a method for removing a compound or ion, such as a phosphorous-containing compound or a phosphorous-containing ion (e.g., phosphate), from the gastrointestinal-tract of an animal by administering an effective amount of crosslinked polyamine particles or a pharmaceutical composition comprising crosslinked polyamine particles, where the crosslinked polyamine particles comprise repeat units represented by the following Formula II:

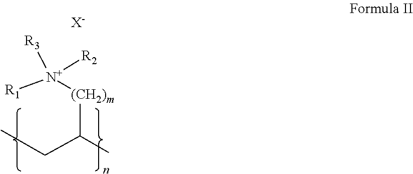

or a copolymer thereof, wherein m is an integer from 0 to 2, such as for example, 0, 1 or 2; n is an integer and each R₁ each R₂ and each R₃ independently represent hydrogen; substituted or unsubstituted, branched or unbranched C₁-C₆ alkyl, such as C₁, C₂, C₃, C₄, C₅ or C₆ alkyl; or substituted or unsubstituted, branched or unbranched C₁-C₆ alkylamino such as C₁, C₂, C₃, C₄, C₅ or C₆ alkylamino; and each X⁻ independently represents a pharmaceutically acceptable counterion, where the crosslinked polyamine particles have one or more of the following characteristics:
a) a particle size distribution such that 75 vol. % or greater of the crosslinked polyallylamine particles have a size of between 250 μm and 4 mm;
b) a particle size distribution where from 5 vol. % to 100 vol. % of the crosslinked polyallylamine particles have a particle size of greater than 500 μm;
c) a particle size distribution such that no more than 20 vol. % of the crosslinked polyallylamine particles have a particle size less than 300 μm;
d) a particle size distribution such that the crosslinked polyamine particles have a d₁₀ value that is between 250 μm and 750 μm
e) a particle size distribution such that the crosslinked polyamine particles have a d₉₀ value that is between 900 μm and 1600 μm;
f) a particle size distribution such that the crosslinked polyamine particles have a d₅₀ between 450 μm and 1100 μm;
g) from 75 wt. % to 100 wt. % of the crosslinked polyamine particles have a mesh size that is −5/+60;
h) from 5 wt. % to 100 wt. % of the crosslinked polyamine particles have a mesh size that is +35;
i) no more than 20 wt. % of the crosslinked polyamine particles have a mesh size that is −50;
j) from 40 wt. % to 60 wt. % of the crosslinked polyamine particles have a mesh size that is −16/+40;
k) a mean gray value greater than. 180;
l) comprises 2 or more constituent particles; and/or
m) a competitive phosphate binding capacity at 60 minutes of greater than 1.2.

In some embodiments, the invention is, consists essentially of, or comprises crosslinked polyamine particles, a pharmaceutical composition comprising crosslinked polyamine particles or a method for removing a compound or ion, such as a phosphorous-containing compound or a phosphorous-containing ion (e.g., phosphate), from the gastrointestinal tract of an animal by administering an effective amount of crosslinked polyamine particles or a pharmaceutical composition comprising crosslinked polyamine particles, where the crosslinked polyamine particles comprise repeat units represented by the following Formula I and/or Formula II:

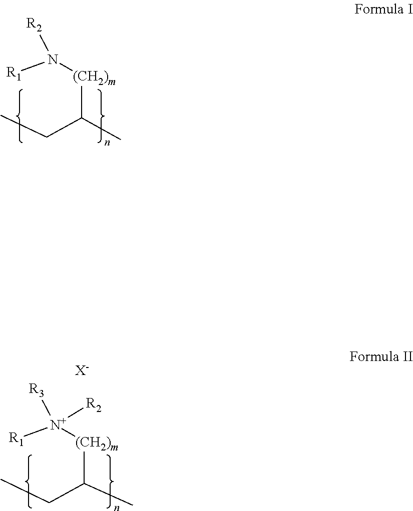

or a copolymer thereof, wherein m is an integer from 0 to 2, such as for example, 0, 1 or 2; n is an integer and each R₁, each R₂ and each R₃ independently represent hydrogen; substituted or unsubstituted, branched or unbranched C₁-C₆ alkyl, such as C₁, C₂, C₃, C₄, C₅ or C₆ alkyl; or substituted or unsubstituted, branched or unbranched C₁-C₆ alkylamino such as C₁, C₂, C₃, C₄, C₅ or C₆ alkylamino; and each X⁻ independently represents a pharmaceutically acceptable counterion, where the crosslinked polyamine particles have one or more of the following characteristics:
a) a particle size distribution such that 75 vol. % or greater of the crosslinked polyallylamine particles have a size of between 250 μm and 4 mm;
b) a particle size distribution where from 5 vol. % to 100 vol. % of the crosslinked polyallylamine particles have a particle size of greater than 500 μm;
c) a particle size distribution such that no more than 20 vol. % of the crosslinked polyallylamine particles have a particle size less than 300 μm;
d) a particle size distribution such that the crosslinked polyamine particles have a d₁₀ value that is between 250 μm and 750 μm
e) a particle size distribution such that the crosslinked polyamine particles have a d₉₀ value that is between 900 μm and 1600 μm;
f) a particle size distribution such that the crosslinked polyamine particles have a d₅₀ between 450 μm and 1100 μm;
g) from 75 wt. % to 100 wt. % of the crosslinked polyamine particles have a mesh size that is −5/+60;
h) from 5 wt% to 100 wt. % of the crosslinked polyamine particles have a mesh size that is +35;
i) no more than 20 wt. % of the crosslinked polyamine particles have a mesh size that is −50;
j) from 40 wt. % to 60 wt. % of the crosslinked polyamine particles have a mesh size that is −16/+40;
k) a mean gray value greater than 180;
l) comprises 2 or more constituent particles; and/or
m) a competitive phosphate binding capacity at 60 minutes of greater than 1.2.

In some embodiments, the invention is, consists essentially of, or comprises crosslinked polyamine particles, a pharmaceutical composition comprising crosslinked polyamine particles or a method for removing a compound or ion, such as a phosphorous-containing compound or a phosphorous-containing ion (e.g. phosphate), from the gastrointestinal tract of an animal by administering an effective amount of crosslinked polyamine particles or a pharmaceutical composition comprising crosslinked polyamine particles, where the crosslinked polyamine particles comprise repeat units represented by the following Formula I and/or Formula II:

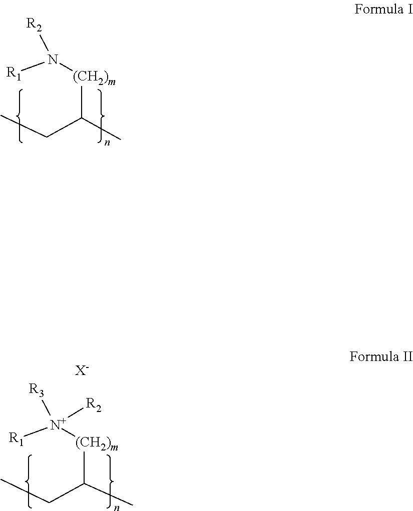

or a copolymer thereof, wherein m is an integer from 0 to 2, such as for example, 0, 1 or 2; n is an integer and each R₁, each R₂ and each R₃ independently represent hydrogen; substituted or unsubstituted, branched or unbranched C₁-C₆ alkyl, such as C₁, C₂, C₃, C₄, C₅ or C₆ alkyl; or substituted or unsubstituted, branched or unbranched C₁-C₆ alkylamino such as C₁, C₂, C₃, C₄, C₅ or C₆ alkylamino; and each X⁻ independently represents carbonate, bicarbonate, hydrochloride or mixtures thereof, where the crosslinked polyamine particles have one or more of the following characteristics:
a) a particle size distribution such that 75 vol. % or greater of the crosslinked polyallylamine particles have a size of between 250 μm and 4 mm;
b) a particle size distribution where from 5 vol. % to 100 vol. % of the crosslinked polyallylamine particles have a particle size of greater than 500 μm;
c) a particle size distribution such that no more than 20 vol. % of the crosslinked polyallylamine particles have a particle size less than 300 μm;
d) a particle size distribution such that the crosslinked polyamine particles have a d₁₀ value that is between 250 μm and 750 μm
e) a particle size distribution such that the crosslinked polyamine particles have a d₉₀ value that is between 900 μm and 1600 μm;
f) a particle size distribution such that the crosslinked polyamine particles have a d₅₀ between 450 μm and 1100 μm;
g) from 75 wt. % to 100 wt. % of the crosslinked polyamine particles have a mesh size that is −5/+60;
h) from 5 wt. % to 100 wt. % of the crosslinked polyamine particles have a mesh size that is +35;
i) no more than 20 wt. % of the crosslinked polyamine particles have a mesh size that is −50;
j) from 40 wt. % to 60 wt. % of the crosslinked polyamine particles have a mesh size that is −161+40;
k) a mean gray value greater than 180;
l) comprises 2 or more constituent particles; and/or
m) a competitive phosphate binding capacity at 60 minutes of greater than 1.2.

In some embodiments, the invention is, consists essentially of, or comprises crosslinked polyamine particles, a pharmaceutical composition comprising crosslinked polyamine particles or a method for removing a compound or ion, such as a phosphorous-containing compound or a phosphorous-containing ion (e.g., phosphate), from the gastrointestinal tract of an animal by administering an effective amount of crosslinked polyamine particles or a pharmaceutical composition comprising crosslinked polyamine particles, where the crosslinked polyamine particles comprising sevelamer hydrochloride represented by the following Formula III:

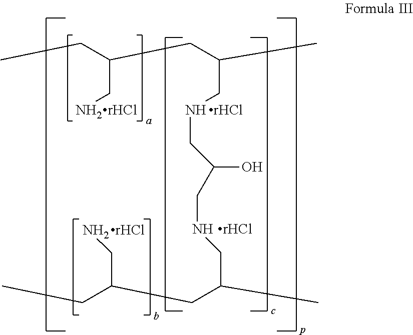

where p is an integer, c is 1, the sum of a and b is 9, and r is 0.4 which represents the fraction of protonated amines, where the crosslinked polyamine particles have one or more of the following characteristics:
a) a particle size distribution such that 75 vol. % or greater of the crosslinked polyallylamine particles have a size of between 250 μm and 4 mm;
b) a particle size distribution where from 5 vol. % to 100 vol. % of the crosslinked polyallylamine particles have a particle size of greater than 500 μm;
c) a particle size distribution such that no more than 20 vol. % of the crosslinked polyallylamine particles have a particle size less than 300 μm;
d) a particle size distribution such that the crosslinked polyamine particles have a d₁₀ value that is between 250 μm and 750 μm
e) a particle size distribution such that the crosslinked polyamine particles have a d₉₀ value that is between 900 μm and 1600 μm;
f) a particle size distribution such that the crosslinked polyamine particles have a d₅₀ between 450 μm and 1100 μm;
g) from 75 wt. % to 100 wt. % of the crosslinked polyamine particles have a mesh size that is −5/+60;
h) from 5 wt. % to ‘100 wt. % of the crosslinked polyamine particles have a mesh size that is +35;
i) no more than 20 wt. % of the crosslinked polyamine particles have a mesh size that is −50;
j) from 40 wt. % to 60 wt. % of the crosslinked polyamine particles have a mesh size that is −16/+40;
k) a mean gray value greater than 180;
l) comprises 2 or more constituent particles; and/or
m) a competitive phosphate binding capacity at 60 minutes of greater than 1.2.

In some embodiments, the invention is, consists essentially of, or comprises crosslinked polyamine particles, a pharmaceutical composition comprising crosslinked polyamine particles or a method for removing a compound or ion, such as a phosphorous-containing compound or a phosphorous-containing ion (e.g., phosphate), from the gastrointestinal tract of an animal by administering an effective amount of crosslinked polyamine particles or a pharmaceutical composition comprising crosslinked polyamine particles, where the crosslinked polyamine particles comprising sevelamer carbonate represented by the following Formula IV:

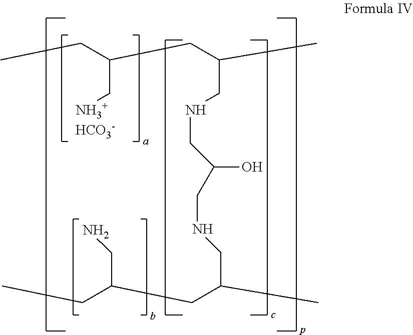

where p is an integer, c is 1, and the sum of a and b is 9, where the crosslinked polyamine particles have one or more of the following characteristics:
a) a particle size distribution such that 75 vol. % or greater of the crosslinked polyallylamine particles have a size of between 250 μm and 4 mm;
b) a particle size distribution where from 5 vol. % to 100 vol. % of the crosslinked polyallylamine particles have a particle size of greater than 500 μm;
c) a particle size distribution such that no more than 20 vol. % of the crosslinked polyallylamine particles have a particle size less than 300 μm;
d) a particle size distribution such that the crosslinked polyamine particles have a d₁₀ value that is between 250 μm and 750 μm
e) a particle size distribution such that the crosslinked polyamine particles have a d₉₀ value that is between 900 μm and 1600 μm;
f) a particle size distribution such that the crosslinked polyamine particles have a d₅₀ between 450 μm and 1100 μm;
g) from 75 wt. % to 100 wt. % of the crosslinked polyamine particles have a mesh size that is −5/+60;
h) from 5 wt. % to 100 wt. % of the crosslinked polyamine particles have a mesh size that is +35;
i) no more than 20 wt. % of the crosslinked polyamine particles have a mesh size that is −50;
j) from 40 wt. % to 60 wt. % of the crosslinked polyamine particles have a mesh size that is −16/+40;
k) a mean gray value greater than 180;
l) comprises 2 or more constituent particles; and/or
m) a competitive phosphate binding capacity at 60 minutes of greater than 1.2.

In some embodiments, the invention is, consists essentially of, or comprises crosslinked polyamine particles, a pharmaceutical composition comprising crosslinked polyamine particles or a method for removing a compound or ion, such as a phosphorous-containing compound or a phosphorous-containing ion (e.g., phosphate), from the gastrointestinal tract of an animal by administering an effective amount of crosslinked polyamine particles or a pharmaceutical composition comprising polyallylamine particles, said polyallylamine particles comprising at least 2 constituent particles of polyallylamine crosslinked with 7-12 wt. % cpichlorohydrin, where the polyallylamine particles have one or more of the following characteristics:
a) a particle size distribution such that 75 vol. % or greater of the crosslinked polyallylamine particles have a size of between 250 μm and 4 mm;
b) a particle size distribution where from 5 vol. % to 100 vol. % of the crosslinked polyallylamine particles have a particle size of greater than 500 μm;
c) a particle size distribution such that no more than 20 vol. % of the crosslinked polyallylamine particles have a particle size less than 300 μm;
d) a particle size distribution such that the crosslinked polyamine particles have a d₁₀ value that is between 250 μm and 750 μm
e) a particle size distribution such that the crosslinked polyamine particles have a d₉₀ value that is between 900 μm and 1600 μm;
f) a particle size distribution such that the crosslinked polyamine particles have a d₅₀ between 450 μm and 1100 μm;
g) from 75 wt. % to 100 wt. % of the crosslinked polyamine particles have a mesh size that is −5/+60;
h) from 5 wt. % to 100 wt. % of the crosslinked polyamine particles have a mesh size that is +35;
i) no more than 20 wt. % of the crosslinked polyamine particles have a mesh size that is −50;
j) from 40 wt. % to 60 wt. % of the crosslinked polyamine particles have a mesh size that is −16/+40;
k) a mean gray value greater than 180;
l) comprises 2 or more constituent particles; and/or
m) a competitive phosphate binding capacity at 60 minutes of greater than 1.2.

In some embodiments, the invention is a method of treating a phosphate imbalance disorder such as hyperphosphatemia comprising administering a therapeutically effective amount of crosslinked polyamine particles of the invention or a composition comprising crosslinked polyamine particles to a patient in need thereof. In some embodiments, the crosslinked polyamine particles described herein may be used in compositions for or methods of controlling serum phosphorus in patients with End Stage Renal Disease (ESRD) or Chronic Kidney Disease (CKD) on hemodialysis. In some embodiments, the crosslinked polyamine particles described herein may be used in compositions for, or methods of, controlling serum phosphorus in patients with End Stage Renal Disease (ESRD) or Chronic Kidney Disease (CKD) that are not on hemodialysis.

In some embodiments, the invention is a method for reducing blood phosphate levels by 5-100%, such as 10%, 20%, 30%, 40%, 50%, 60%, 70%, 80%, or 90% of the elevation above normal blood phosphate levels in a patient in need thereof, the method comprising administering a therapeutically effective amount of crosslinked polyamine particles or composition according to the invention to the patient. In some embodiments, the invention is a method for reducing urinary phosphorous by 5-100%, such as 10%, 20%, 30%, 40%, 50%, 60%, 70%, 80%, or 90% of the elevation above normal urinary phosphate levels in a patient in need thereof, the method comprising administering a therapeutically effective amount of crosslinked polyamine particles or composition according to the invention to the patient.

In some embodiments, the composition includes a mixture of more than one crosslinked polyamine polymer or copolymer of the invention, for example 2-20 such as 2, 3, 4, 5, 6, 7, 8, 9 or 10 polymers or copolymers of the invention.

### Polymerization and Production

In some embodiments, the crosslinked polyamine polymers may be crosslinked in a bulk solution (i.e., using the neat amine polymer and neat crosslinking agents) or in dispersed media. When a bulk process is used, solvents are selected so that they co-dissolve the reactants and do not interfere with the crosslinking reaction. Suitable solvents include water, low boiling alcohols (methanol, ethanol, butanol), acetonitrile, dimethylformamide, dimethylsulfoxide, acetone, methylethylketone, and the like.

Other polymerization methods may include a single polymerization reaction, stepwise addition of individual monomers via a series of reactions, the stepwise addition of blocks of monomers, combinations of the foregoing, or any other method of polymerization, such as, for example, direct or inverse suspension, condensation, phase transfer, emulsion, precipitation techniques, polymerization in aerosol or using bulk polymerization/crosslinking methods and size control processes such as extrusion and grinding. Processes can be carried out as batch, semi-continuous and continuous processes. For processes in dispersed media, the continuous phase can be selected from apolar solvents such as toluene, benzene, hydrocarbon, halogenated solvents, supercritical carbon dioxide, and the like. With a direct suspension process, water can be used, although salt brines are also useful to “salt out” the amine and crosslinking agents in a droplet separate phase.

Examples of some suitable polymerization methods may be found, for example, in the following patents and patent applications each of which is incorporated herein by reference in their entirety: U.S. Pat. Nos. 4,605,701; 5,496,545; 5,618,530; 5,679,717; 5,693,675; 5,702,696; US WO 96/021454; WO 98/057652; EP 7372352; and DE 4227019.

A non-limiting example of polymerization of polyallylamine with epichlorohydrin may occur as follows. Polyallylamine hydrochloride in water may be partially neutralized using a base such as ammonium hydroxide (aqueous ammonia) or NaOH. After neutralization, the polyallylamine may be emulsified with epichlorohydrin using a static or high shear mixer. The resulting oil-in-water emulsion may be polymerized using batch reactor or a single screw or twin screw kneading or LIST reactor. The temperature, amine monomer or amine polymer concentration, ratio of monomeric units to crosslinking agent, rotor speed, and/or work supplied to the reacting polymer may be controlled to help achieve the desired particle size. The polymer leaving the reactor may be suspended in a solvent, such as water, ethanol, ethanol/water mixtures, isopropanol, isopropanol/water mixtures and mixtures thereof followed by filtering and optionally re-suspending one or multiple times, may be milled, wet milled, neutralized and/or protonated using a suitable source such as HCl, CO₂ or carbonic acid, may be milled and/or may be separated before drying using centrifugal force, such as using hydrocyclones or centrifuges. The polymer may be dried using any suitable method such as using a convection oven, a vacuum oven or a fluidized bed and then may be ground, milled and/or sieved or fractionated to a particular desired mesh or particle size after drying. Alternatively, when a solvent that comprises ethanol, ethanol/water mixtures, isopropanol or isopropanollwater mixtures is used, the polymer may not need to be dried prior to grinding, milling and/or sieving or fractionating. In some embodiments, the solvent is water and the polymer is dried prior to grinding.

In some embodiments, after polymerization, the polymer may be hydrated and/or suspended in water, stirred until a gel forms and allowed to cure for a period of time, such as from 30 minutes to 30 hours, from 1 hour to 29 hours, from 3 hours to 28 hours, from 6 hours to 27 hours, from 9 hours to 26 hours, from 12-25 hours, such as 15-21 hours or 17-19 hours. After curing, the gelled polymer may be broken into pieces using any suitable instrument, diluted with water and/or wet milled to a desired constituent particle size. The wet milling may use any known wet milling method and may include using a blender or homogenizer. In some embodiments, after wet milling, or after curing, the gel may be neutralized and/or washed multiple times until the gel (in suspension) has a conductivity of approximately 1 mS/cm³ or less. The polymer may then be protonated, for example carbonated using dry ice, CO₂ and/or carbonic acid or any other suitable carbonating system. After protonation, the gel may be dried using any suitable method such as using a convection oven, a vacuum oven and/or a fluidized bed and then may be ground, milled and/or sieved or fractionated to a particular desired particle or mesh size after drying. Alternatively, when a solvent that comprises ethanol, ethanol/water mixtures, isopropanol or isopropanol/water mixtures is used to wash the gel before or after carbonation, it may not be necessary to dry the gel prior to grinding, milling and/or sieving or fractionating. In some embodiments, the solvent is water and the polymer is dried prior to grinding.

In some embodiments, crosslinked polyamine polymers of the invention may be formed from constituent particles of the crosslinked polyamine, which may be placed in a solvent, such as such as water, ethanol, ethanol/water mixtures, isopropanol, isopropanol/water mixtures and mixtures thereof, dried using any suitable method such as using a convection oven, a vacuum oven or a fluidized bed, and then ground, milled and/or sieved or fractionated to a particular desired particle or mesh size after drying. Alternatively, when a solvent that comprises ethanol, ethanol/water mixtures, isopropanol or isopropanol/water mixtures is used to wash the gel before or after carbonation, it may not be necessary to dry the gel prior to grinding, milling and/or sieving or fractionating. In some embodiments, the solvent is water and the polymer is dried prior to grinding.

In some embodiments, crosslinked polyamine polymers of the invention may be formed using or starting from epichlorohydrin crosslinked polyallylamine carbonate (such as sevelamer carbonate) constituent particles. In some embodiments, epichlorohydrin crosslinked polyallylamine carbonate having an average particles size within the desired constituent particle size range may be suspended in a solvent such as water, ethanol, ethanol/water mixtures, isopropanol, isopropanol/water mixtures and mixtures thereof, stirred until forming a gel. The gel may then be dried for from 30 minutes to 30 hours, such as from 1 hour to 29 hours, from 3 hours to 28 hours, from 6 hours to 27 hours, from 9 hours to 26 hours, from 12-25 hours, such as 15-21 hours or 17-19 hours and the dried gel may then be milled using any suitable milling or grinding equipment and sieved or fractionated to the desired particle size/particle size distribution. A solvent that comprises ethanol, ethanol/water mixtures, isopropanol or isopropanol/water mixtures may be used to wash the gel after curing and it may not be necessary to dry the gel prior to grinding, milling and/or sieving or fractionating. In some embodiments, the solvent is water and the polymer is dried prior to grinding.

In some embodiments, the solvent comprises water. In some embodiments, the solvent comprises an ethanol/water mixture such as from 5 wt. % to 95 wt. % ethanol and from 5 wt. % to 95 wt. % water. In some embodiments, the solvent comprises an isopropanol/water mixture such as from 5 wt. % to 95 wt. % isopropanol and from 5 wt. % to 95 wt. % water.

In some embodiments, the gel may be dried at room temperature. In other embodiments, the gel may be dried at an elevated temperature such as from 30° C. to 65° C. In some embodiments, the gel prior to drying, for example at a % LOD of excess of 35, such as 40, 60, 70, 80, or 85, may optionally be wet co-milled. In some embodiments, the gel may be dried in a forced air oven. In other embodiments, the gel may be dried in a vacuum oven. In other embodiments, the gel may be dried in a fluidized bed. Any suitable drying temperature may be used. In some embodiments, the drying temperature may be from 15° C. to 115° C., such as from 20° C. to 110° C., from 25° C. to 100° C. from 30° C. to 90° C., 35° C. to 80° C., from 40° C. to 75° C., from 45° C. to 65° C. or from 50° C. to 60° C.

In some embodiments, the gel may be dried at room temperature. In other embodiments, the gel may be dried at an elevated temperature such as from 30° C. to 65° C. In some embodiments, the gel prior to drying, for example at a % LOD of excess of 15, such as 20, 25, 30, 35, 40, 50, 60, 70, 80, or 85, may be wet co-milled. In some embodiments, the resulting particles may be dried in a forced air oven. In other embodiments, the resulting particles may be dried in a vacuum oven. In other embodiments, the resulting particles may be dried in a fluidized bed. Any suitable drying temperature may be used. In some embodiments, the drying temperature may be from 15° C. to 115° C., such as from 20° C. to 110° C., from 25° C. to 100° C. from 30° C. to 90° C., 35° C. to 80° C., from 40° C. to 75° C., from 45° C. to 65° C. or from 50° C. to 60° C. In some embodiments, the drying may be accomplished in more than one step for example, the particles may be dried to a % LOD of between 15 and 50, such as 20 and 35, 25 and 40, 25 and 35, or 28 and 32, and then co-milled again before further drying in a secondary dryer. In some embodiments, the secondary dryer may be a forced air oven, a vacuum oven, a fluidized bed or a combination of any of these. Any suitable drying temperature may be used. In some embodiments, the drying temperature may be from 50° C. to 150° C., such as from 70° C. to 140° C., from 80° C. to 130° C. from 90° C. to 120° C., 100° C. to 115° C., or from 105° C. to 112° C. In some embodiments, after the particles are dried to a % LOD of less than 5, such as less than 3, 2, or 1, the particles may be sieved to the desired or specified size and then may be optional further cured. In some embodiments, the sieved particles may be in a forced air oven, a vacuum oven, a fluidized bed or a combination of any of these. Any suitable curing temperature may be used. In some embodiments, the curing temperature may be from 50° C. to 150° C., such as from 70° C. to 140° C., from 80° C. to 130° C. from 90° C. to 120° C., 100° C. to 115° C., or from 105° C. to 112° C. and the duration of curing, which depends in part on the curing temperature (i.e., the higher the curing temperature the shorter the currng time), may be for a time greater than 1 hour, such as between 2 and 8 hours, 3 and 6 hours, 3.5 and 5 hours, or 3.5 and 4.5 hours.

In some embodiments, the polymer or polymer gel may be ground, wet milled and/or milled. Any suitable grinding or milling equiment may be used including manual grinding techniques such as mortar and pestle, potato or other mashers and automated grinding or milling using equiment such as blenders, grinders and mills including coffee grinders, industrial or other commercial blenders. In some embodiments, the polymer or polymer gel may be milled or ground using a jet-mill, a fluidized jet-mill, a pin-mill, a cosmomizer, a cavitation-mill and/or a dispersion mill. Examples of some suitable milling techniques may be found in Lachman et al., The Theory and Practice of Industrial Pharmacy (1986), the entire contents of which is hereby incorporated by reference. In some embodiments, the grinding or milling may be conducted in the presence of various grinding media that may assist in the grinding.

Any suitable method of controlling or achieving the desired particle size may be used. The particle size of the crosslinked polyamine polymers may be controlled by controlling various polymerization process parameters such as temperature, monomer and crosslinker concentration, solvent, monomer to solvent ratio, pH, infusion rate, mixing rate, and by selecting the downstream process and processing parameters. For example, the particle size may be affected by the orifice size of a spray dryer nozzle and the height of a spray drying tower or the drying temperature. In addition, after polymerization, the particles may be further processed to achieve the desired particle size such as ground using a grinder or a mill or selectively sieved. Specific suitable downstream processing methods include, but are not limited to grinding, milling, wet milling, spray drying, sieving, precipitation, suspension or re-suspension and filtration, separation using passive or active centrifugal forces, spray-freezing and any combination thereof.

In some embodiments, the crosslinked polyamine particles may exhibit acid stability. In some embodiments, the acid stability of the crosslinked polyamine particles may be improved by curing the crosslinked polyamine particles, such as by exposing the particles to an elevated temperature or holding the crosslinked polyamine particles at an elevated temperature for an extended period of time. In some embodiments, the acid stability of the crosslinked polyamine particles may be improved by curing the crosslinked polyamine particles, such as by holding the crosslinked polyamine particles at a temperature greater than 35° C., such as greater than 40° C., greater than 45° C., greater than 50° C., greater than 55° C., greater than 60° C., greater than 65° C., greater than 75° C., greater than 85° C., greater than 95° C., greater than 105° C. or greater than 110° C. (but less than a temperature that may cause decomposition of the particles, such as not above 300° C., for example not greater than 250° C. or not greater than 200° C.) for an extended period of time, such as for greater than 1 hour, greater than 2 hours, greater than 3 hours, greater than 4 hours, greater than 7 hours, greater than 10 hours, greater than 15 hours, greater than 20 hours, greater than 24 hours, greater than 36 hours, greater than 2 days, greater than 4 days, greater than 7 days, greater than 10 days, greater than 14 days, greater than 17 days, greater than 20 days, greater than 25 days or greater than 28 days (but less than a procedurally impractical amount of days, such as less than 100 days, for example less than 60 days, or less than 50 days). In one embodiment, the acid stability of the crosslinked polyamine particles may be improved by curing the crosslinked polyamine particles at a temperature of at least 60° C. for at least 3 weeks. In one embodiment, the acid stability of the crosslinked polyamine particles may be improved by curing the crosslinked polyamine particles at a temperature of greater than 100° C., such as 105° C., 110° C. or greater than 110° C. for greater than 1 hour, such as 2 hours, 3 hours, 4 hours or greater than 4 hours. Generally, the curing process is accomplished by heating the particles after they have been dried to a % LOD of less than 5%, such as less than 3%, 2% or 1%. The particles may then be cured by heating the particles for from 30 minutes to 30 hours, such as from 1 hour'to 29 hours, from 3 hours to 28 hours, from 6 hours to 27 hours, from 9 hours to 26 hours, from 12-25 hours, such as 15-21 hours or 17-19 hours at an elevated temperature, for example a temperature greater than 60° C., such as from 70° C. to 180° C., from 75° C. to 150° C. from 80° C. to130° C., 85° C. to 125° C., from 90° C. to 120° C., from 95° C. to 115° C. or from 100° C. to 115° C. The higher the temperature used the lower the time necessary to effectively cure the particles with a concern that a high temperature for a prolonged time period could adversely affect the material and/or the performance of the material. It should be understood that curing may also be accomplished by varying the temperature over time or with step changes to the temperature, for example curing could be initiated at a higher temperature, for example greater than 110° C., and then changed to a lower temperature, for example 110° C. or lower, or vice versa.

In some embodiments, the acid stability of the crosslinked polyamine particles may be improved by wetting constituent particles of the crosslinked polyamine polymers and then heat treating the wetted crosslinked polyamine polymers at an elevated temperature for an extended period of time. In some embodiments, the constituent particles are wetted to form aggregate particles having a % Loss on Drying (% LOD) of greater than 20% LOD, greater than 30% LOD, greater than 40% LOD, greater than 50% LOD, greater than 60% LOD, greater than 70% LOD or greater than 80% LOD. In some embodiments, the stability of the crosslinked polyamine particles may be improved by wetting the crosslinked polyamine particles, such as crosslinked polyamine particles, crosslinked polyamine constituent particles or crosslinked polyamine aggregate particles and then exposing them to an elevated temperature or holding them at an elevated temperature for an extended period of time such as at a temperature greater than 35° C., such as greater than 40° C., greater than 45° C., greater than 50° C., greater than 55° C., greater than 60° C., greater than 65° C., greater than 75° C., greater than 85° C., greater than 95° C., greater than 105° C. or greater than 110° C. (but less than a temperature that may cause decomposition of the particles, such as not above 300° C., for example not greater than 250° C. or not greater than 200° C.) for an extended period of time, such as for greater than 1 hour, greater than 2 hours, greater than 3 hours, greater than 4 hours, greater than 7 hours, greater than 10 hours, greater than 15 hours, greater than 20 hours, greater than 24 hours, greater than 36 hours, greater than 2 days, greater than 4 days, greater than 7 days, greater than 10 days, greater than 14 days, greater than 17 days, greater than 20 days, greater than 25 days or greater than 28 days (but less than a procedurally impractical amount of days, such as less than 100 days, for example less than 60 days, or less than 50 days). In some embodiments, the stability of the crosslinked polyamine particles may be improved by wetting the crosslinked polyamine particles, such as crosslinked polyamine particles, crosslinked polyamine constituent particles or crosslinked polyamine aggregate particles and then heat treating the crosslinked polyamine particles at a temperature of at least 60° C. for at least 3 weeks. In some embodiments, the stability of the crosslinked polyamine particles may be improved by wetting the crosslinked polyamine particles, such as crosslinked polyamine particles, crosslinked polyamine constituent particles or crosslinked polyamine aggregate particles and then heat treating the crosslinked polyamine particles at a temperature of greater than 100° C., such as 105° C., 110° C. or greater than 110° C. for greater than 1 hour, such as 2 hours, 3 hours, 4 hours or greater than 4 hours. In some embodiments, the wetted particles, such as crosslinked polyamine particles, crosslinked polyamine constituent particles or crosslinked polyamine aggregate particles may be kept at the elevated temperature for sufficient time to reduce the % LOD of the particles to less than 20%, such as less than 10%, less than 5%, less than 4%, less than 3%, less than 2.5% or less than 2%. In some embodiments, the yield by weight of crosslinked polyamine particles having a particle size that is −20/+50 mesh may be greater than 15%, greater than 20%, greater than 30%, greater than 40%, greater than 50%, greater than 55%, greater than 60%, greater than 70%, or greater than 80%. The improved stabilty of the particle may include one or more of the following: acid stability, shelf-life stability, dissolution stability, tabletting stability, mechanical stability, compositional stability, and/or conformational stability.

In some embodiments, heat treating the crosslinked polyamine particles or the wetted crosslinked polyamine particles at an elevated temperature for an extended period of time may include holding the particles at one elevated temperature for a portion of the extended period of time and holding the particles at a different (may be higher or lower than the initial elevated temperature) elevated temperature for a second portion of the extended period of time. It should be understood that the number of elevated temperatures and the lengths of time at each elevated temperature at which the particles are held may be varied throughout the elevated temperature range and for different periods of time throughout the extended ranges without departing from these embodiments of the invention.

In some embodiments, the acid stability may be measured by comparing the particle sizes after treatment in acid, such as 0.1-1.5 N HCl, such as 0.2 N HCl, 0.3 N HCl, 0.4 N HCl 0.5 N HCl, 0.6 N HCl, 0.7 N HCl, 0.8 N HCl, 0.9 N HCl, 1.0 N HCl, 1.1 N HCl, 1.2 N HCl, 1.3 N HCl or 1.4 N HCl, of crosslinked polyamine particles that have been cured with acid treated crosslinked polyamine particles that have not been cured. In some embodiments, the acid stability may be measured by comparing the particle size, such as the wet particle size, of crosslinked polyamine particles that have been acid treated shortly after the final sieving step of an embodiments of the preparation process with the particle size, such as the wet particle size, of crosslinked polyamine particles that are acid treated after having been cured, such as at a temperature of greater than 50° C., such as 60° C. for greater than 1 week, such as 2 weeks, 3 weeks or 4 weeks. In some embodiments, the acid stability may be demonstrated by or may comprise a particle size for acid treated crosslinked polyamine particles that have been cured that is greater than 1.2 fold, greater than 1.5 fold, greater than 1.7 fold, greater than 2.0 fold, greater than 2.1 fold or greater than 2.2 fold the particle size of acid treated crosslinked polyamine particles that have not been cured. For example. the acid stability may be demonstrated by comparing the wet particle size, in acid (as measured in accordance with the Wet Particle Size & Distribution test) for cured crosslinked polyamine aggregate particles relative to the wet particle size, in acid, of similar crosslinked polyamine aggregate particles that have not been cured (or prior to curing).

In some embodiments, the acid stability may be measured by comparing the retention of competitive phosphate binding for crosslinked polyamine particles that have been cured prior to acid treatment with crosslinked polyamine particles that have been cured but that have not been acid treated. In some embodiments, the acid stability of the crosslinked polyamine particles may be demonstrated by or may comprise greater than 60% retention of competitive phosphate binding of the acid treated particles relative to the non-acid treated particles, such as greater than 65% retention, greater than 70% retention, greater than 75% retention, greater than 80% retention or greater than 85% retention of competitive phosphate binding relative to non-acid treated particles.

In some embodiments, the cured particles may have volume weighted mean particle size, when measured in accordance with the Wet Particle Size & Distribution (in Acid) test method, of greater than 350 μm, for example greater than 375 μm, greater than 400 μm, greater than 425 μm, greater than 450 μm, greater than 475 μm, or between 425 μm and 750 μm. The particles may additional or alternatively have a volume weighted mean particle size, when measured in accordance with the Wet Particle Size & Distribution (in Phosphate Buffer) test method of greater than 500 μm, for example greater than 525 μm, greater than 550 μm, greater than 575 μm, greater than 600 μm, greater than 625 μm, greater than 650 μm, greater than 675 μm, greater than 700 μm , or between 600 μm and 800 μm.

In some embodiments, the acid stability may be measured by comparing the particle sizes after treatment in acid, such as 0.1-1.5 N HCl, such as 0.2 N HCl, 0.3 N HCl, 0.4 N HCl 0.5 N HCl, 0.6 N HCl, 0.7 N HCl, 0.8 N HCl, 0.9 N HCl, 1.0 N HCl, 1.1 N HCl, 1.2 N HCl, 1.3 N HCl or 1.4 N HCl, of crosslinked polyamine particles that have been cured with acid treated crosslinked polyamine particles that have not been cured. In some embodiments, the acid stability may be measured by comparing the particle size, such as the wet particle size, of crosslinked polyamine particles that have been acid treated shortly after the final sieving step of an embodiments of the preparation process with the particle size, such as the wet particle size, of crosslinked polyamine particles that are acid treated after having been cured, such as at a temperature of greater than 50° C., such as 60° C. for greater than 1 week, such as 2 weeks, 3 weeks or 4 weeks. In some embodiments, the acid stability may be demonstrated by or may comprise a particle size for acid treated crosslinked polyamine particles that have been cured that is greater than 1.2 fold, greater than 1.5 fold, greater than 1.7 fold, greater than 2.0 fold, greater than 2.1 fold or greater than 2.2 fold the particle size of acid treated crosslinked polyamine particles that have not been cured and the cured particles may have volume weighted mean particle size, when measured in accordance with the Wet Particle Size & Distribution (in Acid) test method, of greater than 350 μm, for example greater than 375 μm, greater than 400 μm, greater than 425 μm, greater than 450 μm, greater than 475 μm, or between 425 μm and 750 μm. The particles may additional or alternatively have a volume weighted mean particle size, when measured in accordance with the Wet Particle Size & Distribution (in Phosphate Buffer) test method of greater than 500 μm, for example greater than 525 μm, greater than 550 μm, greater than 575 μm, greater than 600 μm, greater than 625 μm, greater than 650 μm, greater than 675 μm, greater than 700 μm , or between 600 μm and 800 μm.

In some embodiments, the cured particles may have a allylamine ppm value of between 0.4 and 1.0, for example between 0.5 and 0.85, such as 0.6 and 0.75.

In some embodiments, the cured particles may have a %soluble oligomers of less than 1.0%, for example less than 0.5%, such as less than 0.1%, or 0.05%.

In some embodiments, the cured particles may have a True Density of between 1.0 and 2 g/cubic centimeter, for example between 1.0 and 1.5 g/cubic centimeter.

In some embodiments, the cured particles may have a Tap Density of between 0.25 and 1 g/ml, for example between 0.4 and 0.6 g/ml.

In some embodiments, the cured particles may have a Bulk Density of between 0.20 and 0.8 g/ml, for example between 0.2 and 0.4 g/ml.

In some embodiments, the cured particles may have a pH of between 9 and 10, for example between 9.3 and 9.7.

In some embodiments, the cured particles may have a DSC-Glass Transition temperature of between 50° C. and 65° C., for example between 55° C. and 60° C.

In some embodiments, the cured particles may have a competitive phosphate binding of between 1.70 mmol/g and 3.2 mmol/g, for example 1.80 mmol/g and 3.0 mmol/g or 1.90 mmol/g and 2.7 mmol/g or 1.95 mmol/g and 2.5 mmol/g or 1.98 mmol/g and 2.4 mmol/g or 2.0 mmol/g and 2.3 mmol/g.

In some embodiments, the cured particles may have volume weighted mean particle size, when measured in accordance with the Wet Particle Size & Distribution (in Acid) test method, of greater than 350 μm, for example greater than 375 μm, greater than 400 μm, greater than 425 μm, greater than 450 μm, greater than 475 μm, or between 425 μm and 750 μm; a volume weighted mean particle size, when measured in accordance with the Wet Particle Size & Distribution (in Phosphate Buffer) test method of greater than 500 μm, for example greater than 525 μm, greater than 550 μm, greater than 575 μm, greater than 600 μm, greater than 625 μm, greater than 650 μm, greater than 675 μm, greater than 700 μm , or between 600 μm and 800 μm; an allylamine ppm value of between 0.4 and 1.0, for example between 0.5 and 0.85, such as 0.6 and 0.75; a % soluble oligomers of less than 1.0%, for example less than 0.5%, such as less than 0.1%, or 0.05%; a True Density of between 1.0 and 2 g/cubic centimeter, for example between 1.0 and 1.5 g/cubic centimeter; a Tap Density of between 0.25 and 1 g/ml, for example between 0.4 and 0.6 g/ml; a Bulk Density of between 0.20 and 0.8 g/ml, for example between 0.2 and 0.4 g/ml; a pH of between 9 and 10, for example between 9.3 and 9.7; a DSC-Glass Transition temperature of between 50° C. and 65° C., for example between 55° C. and 60° C.; and/or a competitive phosphate binding of between 1.70 mmol/g and 3.2 mmol/g, for example 1.80 mmol/g and 3.0 mmol/g or 1.90 mmol/g and 2.7 mmol/g or 1.95 mmol/g and 2.5 mmol/g or 1.98 mmol/g and 2.4 mmol/g or 2.0 mmol/g and 2.3 mmol/g.

In some embodiments, tablets composed of the polyamine particles described herein may have a Volume Weighted Mean, as measured in accordance with the Tablet Dissolution Particle Size & Distribution (in Acid) test method, of greater than 250 μm, for example greater than 275 g,m, greater than 300 μm, greater than 325 μm, greater than 350 μm, greater than 375 μm, greater than 400 μm, greater than 425 μm, greater than 450 μm, or between 325 μm and 550 μm.

In some embodiments, tablets composed of the polyamine particles described herein may have a Volume Weighted Mean, as measured in accordance with the Tablet Dissolution Particle Size & Distribution (in Phosphate Buffer) test method, of greater than 325 μm, for example greater than 350 μm, greater than 375 μm, greater than 400 μm, greater than 425 μm, greater than 450 μm, greater than 475 μm, greater than 500 μm, greater than 525 μm, or between 400 μm and 625 μm.

In some embodiments, tablets composed of the polyamine particles described herein may have a Volume % Mode, as measured in accordance with the Tablet Dissolution Particle Size & Distribution (in Phosphate Buffer) test method, of greater than 300, for example greater than 350, greater than 375, greater than 400, greater than 425, greater than 450, greater than 475, greater than 500, greater than 525, or between 475 and 625.

In some embodiments, tablets composed of the polyamine particles described herein may have a Volume Weighted Mean, as measured in accordance with the Tablet Dissolution Particle Size & Distribution (in Acid) test method, of greater than 250 μm, for example greater than 275 μm, greater than 300 μm, greater than 325 μm, greater than 350 μm, greater than 375 μm, greater than 400 μm, greater than 425 μm, greater than 450 μm, or between 325 μm and 550 μm; Volume Weighted Mean, as measured in accordance with the Tablet Dissolution Particle Size & Distribution (in Phosphate Buffer) test method, of greater than 325 μm, for example greater than 350 μm, greater than 375 μm, greater than 400 μm, greater than 425 μm, greater than 450 μm, greater than 475 μm, greater than 500 μm, greater than 525 μm, or between 400 μm and 625 μm; and/or Volume % Mode, as measured in accordance with the Tablet Dissolution Particle Size & Distribution (in Phosphate Buffer) test method, of greater than 300, for example greater than 350, greater than 375, greater than 400, greater than 425, greater than 450, greater than 475, greater than 500, greater than 525, or between 475 μm and 625 μm.

The particles may additional or alternatively have a volume weighted mean particle size, when measured in accordance with the Wet Particle Size & Distribution (in Phosphate Buffer) test method of greater than 500 μm, for example greater than 525 μm, greater than 550 μm, greater than 575 μm, greater than 600 μm, greater than 625 μm, greater than 650 μm, greater than 675 μm, greater than 700 μm , or between 600 μm and 800 μm.

In some embodiments, prior to crosslinking, the amine polymers may be copolymers. In some embodiments, the copolymers may comprise a monomer comprising a compound having at least one unit according to any of Formulas I-II which is copolymerized with one or more other comonomers or oligomers or other polymerizable groups. The amine polymers and copolymers may be crosslinked, may have crosslinking or other linking agents or monomers within the polymer backbone or as pendant groups or may be formed or polymerized to form a polymer network or mixed polymer network comprising: amine monomers or residues thereof, amine polymers or residues thereof, crosslinking agents or residues thereof, or other linking agents or residues thereof. The network may include multiple connections between the same or different molecules that may be direct or may include one or more linking groups such as crosslinking agents or other linking agents such as monomers or oligomers or residues thereof.

Non-limiting examples of suitable comonomers which may be used alone or in combination to form the copolymers include: styrene, substituted styrene, alkyl acrylate, substituted alkyl acrylate, alkyl methacrylate, substituted alkyl methacrylate, acrylonitrile, methacrylonitrile, acrylamide, methacrylamide, N-alkylacrylamide, N-alkylmethacrylamide, N,N-dialkylacrylamide, N,N-dialkylmethacrylamide, isoprene, butadiene, ethylene, vinyl acetate, N-vinyl amide, maleic acid derivatives, vinyl ether, allyl, methallyl monomers and combinations thereof. Functionalized versions of these monomers may also be used. Additional specific monomers or comonomers that may be used in this invention include, but are not limited to, methyl methacrylate, ethyl methacrylate, propyl methacrylate (all isomers), butyl methacrylate (all isomers), 2-ethylhexyl methacrylate, isobornyl methacrylate, methacrylic acid, benzyl methacrylate, phenyl methacrylate, methyl acrylate, ethyl acrylate, propyl acrylate (all isomers), butyl acrylate (all isomers), 2-ethylhexyl acrylate, isobornyl acrylate, acrylic acid, benzyl acrylate, phenyl acrylate, acrylonitrile, styrene, N,N-dimethylaminoethyl methacrylate, N,N-diethylaminoethyl methacrylate, N,N-dimethylaminoethyl acrylate, N,N-diethylaminoethyl acrylate, maleic anhydride, allylamine, methallylamine, allylalcohol, butadiene, isoprene, chloroprene, ethylene, vinyl acetate and combinations thereof.

In addition, the crosslinked polyamine polymers of the invention may comprise copolymers having any combination of repeat units according to Formulas I-II.

In some embodiments, crosslinked polyamine particles of the invention may not dissolve in solvents, and, at most, swell in solvents. The swelling ratio may be calculated according to the procedure in the Test Methods section below and is typically in the range of about 1 to about 150, such as about 2.5 to about 150, about 5 to about 150, about 5 to about 100, about 5 to about 80, about 5 to about 60, about 5 to about 40, or about 5 to about 20; for example, 1 to 20, 2.5 to 19, 5 to 18, 5 to 16 or 5 to 15, such as greater than 1 and less than 50, greater than 2.5 and less than 45, greater than 5 and less than 40, greater than 5 and less than 20, greater than 9 and less than 20, greater than 11 and less than 20, such as 1, 2, 3, 4, 5, 6, 7, 8, 9, 10, 11, 12, 13, 14, 15, 16, 17, 18, 19 or more.

Crosslinking agents are typically compounds having at least two functional groups that are selected from a halogen group, carbonyl group, epoxy group, ester group, acid anhydride group, acid halide group, isocyanate group, vinyl group, and chloroformate group. The crosslinking agent may be attached to the carbon backbone or to a nitrogen of an amine polymer, amine monomer or residue thereof.

Examples of crosslinking agents that are suitable for synthesis of the crosslinked polyamine particles of the present invention include, but are not limited to, one or more multifunctional crosslinking agents such as: dihaloalkanes, haloalkyloxiranes, alkyloxirane sulfonates, di(haloalkyl)amines, tri(haloalkyl)amines, diepoxides, triepoxides, tetraepoxides, bis(halomethyl) benzenes, tri(halomethyl) benzenes, tetra(halomethyl) benzenes, epihalohydrins such as epichlorohydrin and epibromohydrin, poly(epichlorohydrin), (iodomethyl)oxirane, bromo-1,2-epoxybutane, 1,2-dibromoethane, 1,3-dichloropropane, 1,2-dichloroethane, 1-bromo-2-chloroethane, 1,3-dibromopropane, bis(2-chloroethyl)amine, tris(2-chloroethyl)amine, and bis(2-chloroethyl)methylamine, 1,3-butadiene diepoxide, 1,5-hexadiene diepoxide, methyl acrylate and the like. When the crosslinking agent is an alkylhalide compound, a base may be used to scavenge the acid formed during the reaction. Inorganic or organic bases are suitable. NaOH is preferred. The base to crosslinking agent ratio may be between about 0.5 to about 2.

In some embodiments, the crosslinking agents may be used in the crosslinking reaction in an amount of from 7 wt. % to 12 wt, such as from about 8 wt. % to 11 wt. %, from about 9 wt. % to about 10.4 wt. % or from about 9.4 wt. % to about 10.2 wt. %, such as 8, 9, 9.4, 9.8 or 10 wt. %.

In some embodiments, the weight averaged molecular weight of the polymers and copolymers may be typically at least about 1000. For example, the molecular weight may be from about 1000 to about 1,000,000, such as about 2000 to about 750,000, about 3000 to about 500,000, about 5000 to about 250,000, about 10000 to about 100,000, such as from 15,000-80,000, 20,000 to 75,000, 25,000 to 60,000, 30,000 to 50,000, or 40,000 to 45,000.

The crosslinked polyamine polymers of some embodiments may be formed using a polymerization initiator. Generally, any initiator may be used including cationic and radical initiators. Some examples of suitable initiators that may be used include: the free radical peroxy and azo type compounds, such as azodiisobutyronitrile, azodiisovaleronitrile, dimethylazodiisobutyrate, 2,2′-azobis(isobutyronitrile), 2,2′-azobis(N,N′-dimethyleneisobutyramidine)dihydrochloride, 2,2′-azobis(2-amidinopropane)dihydro chloride, 2,2′-azobis(N,N′-dimethyleneisobutyramidine), 1,1′-azobis(1-cyclohexanecarbo-nitrile), 4,4′-azobis(4-cyanopentanoic acid), 2,2′-azobis(isobutyramide) dihydrate, 2,2′-azobis(2-methylpropane), 2,2′-azobis(2-methylbutyronitrile), VAZO 67, cyanopentanoic acid, the peroxy pivalates, dodecylbenzene peroxide, benzoyl peroxide, di-t-butyl hydroperoxide, t-butyl peracetate, acetyl peroxide, dicumyl peroxide, cumyl hydroperoxide, dimethyl bis(butylperoxy) hexane.

In some embodiments, any of the nitrogen atoms within the crosslinked polyamine particles according to embodiments of the invention may optionally be quaternized to yield the corresponding positively charged tertiary nitrogen group, such as for example, an ammonium or substituted ammonium group. Any one or more of the nitrogen atoms in the crosslinked amine polymers may be quaternized and such quaternization, when present, is not limited to or required to include terminal amine nitrogen atoms. In some embodiments, this quatemization may result in additional network formation and may be the result of addition of crosslinking, linking or amine reactive groups to the nitrogen. The ammonium groups may be associated with a pharmaceutically acceptable counterion.

In some embodiments, crosslinked polyamine particles of the invention may be partially or fully quaternized, including protonated, with a pharmaceutically acceptable counterion, which may be organic ions, inorganic ions, or a combination thereof. Examples of some suitable inorganic ions include halides (e.g., chloride, bromide or iodide) carbonates, bicarbonates, sulfates, bisulfates, hydroxides, nitrates, persulfates and sulfites. Examples of some suitable organic ions include acetates, ascorbates, benzoates, citrates, dihydrogen citrates, hydrogen citrates, oxalates, succinates, tartrates, taurocholates, glycocholates, and cholates. Preferred counterions include chlorides and carbonates.

In some embodiments, crosslinked polyamine particles of the invention may be protonated such that the fraction of protonated nitrogen atoms is from 1% to 100%, such as 10% to 75%, 20% to 60%, 25%% to 55%, 30% to 50%, 35% to 45% or about 40%.

In one embodiment, the pharmaceutically acceptable crosslinked polyamine particles are in partially or fully protonated form and comprise a carbonate anion. In one embodiment, the pharmaceutically acceptable crosslinked polyamine particles are in partially or fully protonated form and comprise a mixture of carbonate and bicarbonate counterions.

In some embodiments, crosslinked polyamine particles of the invention are characterized by their ability to bind compounds or ions. Preferably the crosslinked polyamine particles of the invention bind anions, more preferably they bind organophosphates, phosphate and/or oxalate, and most preferably they bind phosphate. For illustration, anion-binding crosslinked polyamine particles and especially organophosphate or phosphate-binding crosslinked polyamine particles will be described; however, it is understood that this description applies equally, with appropriate modifications that will be apparent to those of skill in the art, to other ions, compounds and solutes. While not wishing to be bound by any theory, crosslinked polyamine particles are believed to bind an ion, e.g., an anion, when they associate with the ion, generally though not necessarily in a noncovalent manner, with sufficient association strength that at least a portion of the ion remains bound under the in vitro or in vivo conditions in which the polymer is used for sufficient time to effect a removal of the ion from solution or from the body. A target ion may be an ion to which the crosslinked polyamine particles bind, and usually refers to the ion whose binding to the crosslinked polyamine particles is thought to produce the therapeutic effect of the crosslinked polyamine particles and may be an anion or a cation. Crosslinked polyamine particles of the invention may have more than one target ion.

For example, some of the crosslinked polyamine particles described herein exhibit organophosphate or phosphate binding properties. Phosphate binding capacity is a measure of the amount of phosphate ion a phosphate binder can bind in a given solution. Some embodiments of the crosslinked polyamine particles of the invention have an in vitro non-competitive phosphate binding capacity which is greater than about 0.2, 0.4, 0.5, 1.0, 1.2, 1.3, 1.4, 1.5, 1.6, 1.7, 1.8, 2.0, 2.5, 3.0, 3.5, 4.0, 4.5, 5.0, 6.0, 8.0, 10.0, greater than about 12, or up to about 14, mmol/g. In some embodiments, the in vitro non-competitive phosphate binding capacity of crosslinked polyamine particles of the invention is greater than about 0.4 mmol/g, greater than about 2.5 mmol/g, greater than about 3 mmol/g, greater than about 4.5 mmol/g or greater than about 6 mmol/g. In some embodiments, the in vitro non-competitive phosphate binding capacity can be between about 0.2 mmol/g and about 14 mmol/g, such as between about 0.4 mmol/g and about 10 mmol/g, between about 1.0 mmol/g and about 8 mmol/g, between about 1.5 mmol/g and about 8 mmol/g, between about 2.0 mmol/g and about 8 mmol/g, between about 2.5 mmol/g and about 8 mmol/g, between about 3 mmol/g and about 6 mmol/g or between about 3 mmol/g and about 5 mmol/g. The in vitro non-competitive phosphate binding capacity may be measured according to the techniques described in the Test Methods section below.

In some embodiments, the crosslinked polyamine particles according to the invention have an in vitro competitive phosphate binding capacity of between 0.4 mmol/g and 10 mmol/g, for example between 0.5 mmol/g and 7 mmol/g, between 0.6 mmol/g and 5 mmol/g, between 0.7 mmol/g and 4 mmol/g or between 0.8 mmol/g and 2.5 mmol/g throughout a physiologically significant time period. A physiologically significant time period may be the length of time during which significant uptake of a target ion occurs in a human. For example, for phosphate the physiologically significant time period may be from 0 to 5 hours, such as 0.5 to 5 hours, 1 to 4.5 hours, 1.5 to 4 hours, 2 to 3.5 hours or 1, 1.5, 2, 2.5, 3, 3.5, 4, 4.5 or 5 hours. The in vitro competitive phosphate binding capacity may be measured according to the techniques described in the Test Methods section below.

In some embodiments, the crosslinked polyamine particles of the present invention have an in vitro non-competitive phosphate binding capacity at 5 hours that is within 20%, for example within 15%, 12.5%, 10% or even 5% of that of RENAGEL.

In some embodiments, the crosslinked polyamine particles of the present invention have an in vitro competitive phosphate binding capacity of less than 1.4 mmol/g, such as less than 1.3 mmol/g, less than 1.2 mmol/g, less than 1.1 mmol/g of phosphate after 20 minutes. In some embodiments, the crosslinked polyamine particles have an in vitro competitive phosphate binding capacity less than 1.4 mmol/g of phosphate after 20 minutes and greater than 0.4 mmol/g, such as greater than 0.5 mmol/g, greater than 0.6 mmol/g, greater than 0.7 mmol/g or greater than 0.8 mmol/g after 5.0 hours.

In some embodiments, the crosslinked polyamine particles of the invention have a competitive phosphate binding capacity of between 0.4 mmol/g and 1.4 mmol/g, such as between 0.4 mmol/g and 1.2 mmol/g, between 0.45 mmol/g and 1.1 mmol/g, between 0.5 mmol/g and 1.0 mmol/g, between 0.6 mmol/g and 0.9 mmol/g or between 0.7 mmol/g and 0.8 mmol/g at one hour or 60 minutes and/or a competitive binding capacity of between 0.4 nunol/g and 1.0 mmol/g, such as between 0.4 mmol/g and 0.9 mmol/g, between 0.5 mmol/g and 0.8 mmol/g, between 0.55 mmol/g and 0.75 mmol/g at 5 hours.

In some embodiments, the crosslinked polyamine particles of the present invention have an in vitro competitive phosphate binding capacity at 1 hour of greater than 20%, for example greater than 30%, greater than 35%, greater than 40% or greater than 45% of the 5 hour or 300 minute in vitro non-competitive phosphate binding capacity of said polymer.

In some embodiments, the crosslinked polyamine particles of the invention have an in vivo phosphate binding capacity of between 0.2 mmol/g and 14 mmol/g, such as between 0.3 mmol/g and 14 mmol/g, between 0.4 mmol/g and 12.5 mmol/g, between 0.5 mmol/g and 10 mmol/g, between 0.75 mmol/g and 8 mmol/g, between 1.0 mmol/g and 6 mmol/g, between 1.25 mmol/g and 5 mmol/g, between 1.5 mmol/g and 4.5 mmol/g, between 2.0 mmol/g and 4.0 mmol/g or between 2.5 mmol/g and 3.5 mmol/g. The in vivo phosphate binding capacity may be measured in any animal, such as any mammal, such as humans or rats. The test methods detail a procedure for measuring the in vivo phosphate binding capacity in rats, which may be suitably modified as appropriate for measurement in humans.

In some embodiments, the crosslinked polyamine particles of the invention have an in vitro bile acid binding capacity of between 0.5 mmol/g and 14 mmol/g, such as between 0.3 mmol/g and 14 mmol/g, between 0.4 mmol/g and 12.5 mmol/g, between 0.5 mmol/g and 10 mmol/g, between 0.75 mmol/g and 8 mmol/g, between 1.0 mmol/g and 6 mmol/g, between 1.25 mmol/g and 6 mmol/g, between 1.5 mmol/g and 6 mmol/g, between 2.0 mmol/g and 6 mmol/g or between 2.5 mmol/g and 6 mmol/g, such as greater than 1.00, 1.5, 2.0, 2.5, 3.0, 3.5, 4, 4.5, 5.0, 5.5, 6.0, 7.0, 8.0, 9.0, 10.0, 11.0, 12.0 or greater than 13.0 mmol/g. The in vitro bile acid binding capacity may be determined according to the procedure detailed in the Test Procedures.

In some embodiments, the crosslinked polyamine particles of the invention have an in vivo bile acid binding capacity of between 0.5 mmol/g and 14 mmol/g, such as between 0.3 mmol/g and 14 mmol/g, between 0.4 mmol/g and 12.5 mmol/g, between 0.5 mmol/g and 10 mmol/g, between 0.75 mmol/g and 8 mmol/g, between 1.0 mmol/g and 6 mmol/g, between 1.25 mmol/g and 6 mmol/g, between 1.5 mmol/g and 6 mmol/g, between 2.0 mmol/g and 6 mmol/g or between 2.5 mmol/g and 6 mmol/g, such as greater than 1.00, 1.5, 2.0, 2.5, 3.0, 3.5, 4, 4.5, 5.0, 5.5, 6.0, 7.0, 8.0, 9.0, 10.0, 11.0, 12.0 or greater than 13.0 mmol/g. The in vivo bile acid binding capacity may be measured in any animal, such as any mammal, such as humans or rats. The test methods detail a procedure for measuring the in vivo bile acid binding capacity in rats, which may be suitably modified as appropriate for measurement in humans.

In some embodiments, crosslinked polyamine particles and compositions of the invention may reduce urinary phosphorous of a patient in need thereof by 5 -100% of the elevation above normal urinary phosphorous levels, such as 10-75%, 25-65%, or 45-60%. Some embodiments may reduce urinary phosphorous by greater than 10%, greater than 20%, greater than 30%, greater than 40%, greater than 45%, greater than 50% or greater than 60% of the elevation above normal urinary phosphorous levels.

In some embodiments, crosslinked polyamine particles and compositions of the invention may reduce blood phosphate of a patient in need thereof by 5 -100% of the elevation above normal blood phosphate levels, such as 10-75%, 25-65%, or 45-60% of the elevation above normal blood phosphate levels. Some embodiments may reduce blood phosphate levels by greater than 10%, greater than 20%, greater than 30%, greater than 40%, greater than 45%, greater than 50% or greater than 60% of the elevation above normal blood phosphate levels.

In some embodiments, the crosslinked polyamine particles comprise polyallylamine crosslinked with 9.0 wt. % to 10 wt. %, such as 9.5 wt. % to 10 wt. % epichlorohydrin where the crosslinked polyamine particles are in the form of a base and/or a hydrochloride or carbonate salt and have a particle size distribution such that 90 vol. % or greater of the crosslinked polyamine particles have a size between 300 μm and 2000 μm, a particle size distribution where greater than 50 vol. % of the crosslinked polyamine particles have a particle size of between 500 μm and 1500 μm, a particle size distribution such that no more than 10 vol. % of the crosslinked polyamine particles have a particle size less than 375 μm, a particle size distribution such that the crosslinked polyamine particles have a d₁₀ value that is between 350 μm and 650 μm, a particle size distribution such that the crosslinked polyamine particles have a d₉₀ value that is between 1100 μm and 1400 μm, a particle size distribution such that the crosslinked polyamine particles have a d₅₀ between 675 μm and 1000 μm, a particle size distribution such that 75 vol. % to 100 vol. % of the crosslinked polyamine particles have a mesh size that is −14/+50, a particle size distribution where greater than 50 vol. % of the crosslinked polyamine particles have a mesh size that is −12/+35, a particle size distribution such that no more than 10 vol. % of the crosslinked polyamine particles have a mesh size that is −45 and/or a particle size distribution such that the crosslinked polyamine particles have an average mesh size of −18/+30.

In some embodiments, the crosslinked polyamine particles comprise polyallylamine crosslinked with 9.0 wt. % to 10 wt. %, such as 9.5 wt. % to 10 wt. % epichlorohydrin where the crosslinked polyamine particles are in the form of a base and/or a hydrochloride or carbonate salt and have a mean gray value greater than 190.

In some embodiments, the crosslinked polyamine particles comprise polyallylamine crosslinked with 9.0 wt. % to 10 wt. %, such as 9.5 wt. % to 10 wt. % epichlorohydrin where the crosslinked polyamine particles are in the form of a base and/or a hydrochloride or carbonate salt and comprise 500 to 1000 constituent particles, the constituent particles having a d₁₀ value between 20 μm and 70 μm, a d₉₀ value between 150 μm and 400 μm and/or a d₅₀ of between 70 μm and 120 μm.

In some embodiments, the crosslinked polyamine particles comprise polyallylamine crosslinked with 9.0 wt. % to 10 wt. %, such as 9.5 wt. % to 10 wt. % epichlorohydrin where the crosslinked polyamine particles are in the form of a base and/or a hydrochloride or carbonate salt and comprise a competitive phosphate binding capacity at 60 minutes of greater than 1.2 mmol/g.

In some embodiments, the crosslinked polyamine particles comprise polyallylamine crosslinked with 9.0 wt. % to 10 wt. %, such as 9.5 wt. % to 10 wt. % epichlorohydrin where the crosslinked polyamine particles are in the form of a base and/or a hydrochloride or carbonate salt and have a particle size distribution such that 90 vol. % or greater of the crosslinked polyamine particles have a size between 300 μm and 2000 μm, a particle size distribution where greater than 50 vol. % of the crosslinked polyamine particles have a particle size of between 500 μm and 1500 μm, a particle size distribution such that no more than 10 vol. % of the crosslinked polyamine particles have a particle size less than 375 μm, a particle size distribution such that the crosslinked polyamine particles have a d₁₀ value that is between 350 μm and 650 μm, a particle size distribution such that the crosslinked polyamine particles have a d₉₀ value that is between 1100 μm and 1400 μm, a particle size distribution such that the crosslinked polyamine particles have a d₅₀ between 675 μm and 1000 μm, a particle size distribution such that 75 vol. % to 100 vol. % of the crosslinked polyamine particles have a mesh size that is −14/+50, a particle size distribution where greater than 50 vol. % of the crosslinked polyamine particles have a mesh size that is -12/+35, a particle size distribution such that no more than 10 vol. % of the crosslinked polyamine particles have a mesh size that is −45 and/or a particle size distribution such that the crosslinked polyamine particles have an average mesh size of −18/+30, where the crosslinked polyamine particles further comprise a mean gray value of greater than 190.

In some embodiments, the crosslinked polyamine particles comprise polyallylamine crosslinked with 9.0 wt. % to 10 wt. %, such as 9.5 wt. % to 10 wt. % epichlorohydrin where the crosslinked polyamine particles are in the form of a base and/or a hydrochloride or carbonate salt and have a particle size distribution such that 90 vol. % or greater of the crosslinked polyamine particles have a size between 300 μm and 2000 μm, a particle size distribution where greater than 50 vol. % of the crosslinked polyamine particles have a particle size of between 500 μm and 1500 μm, a particle size distribution such that no more than 10 vol. % of the crosslinked polyamine particles have a particle size less than 375 μm, a particle size distribution such that the crosslinked polyamine particles have a d₁₀ value that is between 350 μm and 650 μm, a particle size distribution such that the crosslinked polyamine particles have a d₉₀ value that is between 1100 μm and 1400 μm, a particle size distribution such that the crosslinked polyamine particles have a d₅₀ between 675 μm and 1000 μm, a particle size distribution such that 75 vol. % to 100 vol. % of the crosslinked polyamine particles have a mesh size that is −14/+50, a particle size distribution where greater than 50 vol. % of the crosslinked polyamine particles have a mesh size that is −12/+35, a particle size distribution such that no more than 10 vol. % of the crosslinked polyamine particles have a mesh size that is −45 and/or a particle size distribution such that the crosslinked polyamine particles have an average mesh size of −18/+30, where the crosslinked polyamine particles further comprise or are formed from 500 to 1000 constituent particles, the constituent particles having a d₁₀ value between 20 μm and 70 μm, a d₉₀ value between 150 μm and 400 μm and/or a d₅₀ of between 70 μm and 120 μm.

In some embodiments, the crosslinked polyamine particles comprise polyallylamine crosslinked with 9.0 wt. %.to 10 wt. %, such as 9.5 wt. % to 10 wt. % epichlorohydrin where the crosslinked polyamine particles are in the form of a base and/or a hydrochloride or carbonate salt and have a particle size distribution such that 90 vol. % or greater of the crosslinked polyamine particles have a size between 300 μm and 2000 μm, a particle size distribution where greater than 50 vol. % of the crosslinked polyamine particles have a particle size of between 500 μm and 1500 μm, a particle size distribution such that no more than 10 vol. % of the crosslinked polyamine particles have a particle size less than 375 μm, a particle size distribution such that the crosslinked polyamine particles have a d₁₀ value that is between 350 μm and 650 μm, a particle size distribution such that the crosslinked polyamine particles have a d₉₀ value that is between 1100 μm and 1400 μm, a particle size distribution such that the crosslinked polyamine particles have a d₅₀ between 675 μm and 1000 μm, a particle size distribution such that 75 vol. % to 100 vol. % of the crosslinked polyamine particles have a mesh size that is −14/+50, a particle size distribution where greater than 50 vol. % of the crosslinked polyamine particles have a mesh size that is −12/+35, a particle size distribution such that no more than 10 vol. % of the crosslinked polyamine particles have a mesh size that is −45 and/or a particle size distribution such that the crosslinked polyamine particles have an average mesh size of −18/+30, where the crosslinked polyamine particles further comprise a competitive phosphate binding capacity at 60 minutes of greater than 1.2 mmol/g.

In some embodiments, the crosslinked polyamine particles comprise polyallylamine crosslinked with 9.0 wt. % to 10 wt. %, such as 9.5 wt. % to 10 wt. % epichlorohydrin where the crosslinked polyamine particles are in the form of a base and/or a hydrochloride or carbonate salt and have a particle size distribution such that 90 vol. % or greater of the crosslinked polyamine particles have a size between 300 μm and 2000 μm, a particle size distribution where greater than 50 vol. % of the crosslinked polyamine particles have a particle size of between 500 μm and 1500 μm, a particle size distribution such that no more than 10 vol. % of the crosslinked polyamine particles have a particle size less than 375 μm, a particle size distribution such that the crosslinked polyamine particles have a d₁₀ value that is between 350 μm and 650 μm, a particle size distribution such that the crosslinked polyamine particles have a d₉₀ value that is between 1100 μm and 1400 μm, a particle size distribution such that the crosslinked polyamine particles have a d₅₀ between 675 μm and 1000 μm, a particle size distribution such that 75 vol. % to 100 vol. % of the crosslinked polyamine particles have a mesh size that is −14/+50, a particle size distribution where greater than 50 vol. % of the crosslinked polyamine particles have a mesh size that is −12/+35, a particle size distribution such that no more than 10 vol. % of the crosslinked polyamine particles have a mesh size that is −45 and/or a particle size distribution such that the crosslinked polyamine particles have an average mesh size of −18/+30, where the crosslinked polyamine particles further comprise a mean gray value of greater than 190 and 500 to 1000 constituent particles, the constituent particles having a d₁₀ value between 20 μm and 70 μm, a d₉₀ value between 150 μm and 400 μm and/or a d₅₀ of between 70 μm and 120 μm.

In some embodiments, the crosslinked polyamine particles comprise polyallylamine crosslinked with 9.0 wt. % to 10 wt. %, such as 9.5 wt. % to 10 wt. % epichlorohydrin where the crosslinked polyamine particles are in the form of a base and/or a hydrochloride or carbonate salt and have a particle size distribution such that 90 vol. % or greater of the crosslinked polyamine particles have a size between 300 μm and 2000 μm, a particle size distribution where greater than 50 vol. % of the crosslinked polyamine particles have a particle size of between 500 μm and 1500 μm, a particle size distribution such that no more than 10 vol. % of the crosslinked polyamine particles have a particle size less than 375 μm, a particle size distribution such that the crosslinked polyamine particles have a d₁₀ value that is between 350 μm and 650 μm, a particle size distribution such that the crosslinked polyamine particles have a d₉₀ value that is between 1100 μm and 1400 μm, a particle size distribution such that the crosslinked polyamine particles have a d₅₀ between 675 μm and 1000 μm, a particle size distribution such that 75 vol. % to 100 vol. % of the crosslinked polyamine particles have a mesh size that is −14/+50, a particle size distribution where greater than 50 vol. % of the crosslinked polyamine particles have a mesh size that is −12/+35, a particle size distribution such that no more than 10 vol. % of the crosslinked polyamine particles have a mesh size that is −45 and/or a particle size distribution such that the crosslinked polyamine particles have an average mesh size of −18/+30, where the crosslinked polyamine particles further comprise a mean gray value of greater than 190 and a competitive phosphate binding capacity at 60 minutes of greater than 1.2 mmol/g.

In some embodiments, the crosslinked polyamine particles comprise polyallylamine crosslinked with 9.0 wt. % to 10 wt. %, such as 9.5 wt. % to 10 wt. % epichlorohydrin where the crosslinked polyamine particles are in the form of a base and/or a hydrochloride or carbonate salt and have a particle size distribution such that 90 vol. % or greater of the crosslinked polyamine particles have a size between 300 μm and 2000 μm, a particle size distribution where greater than 50 vol. % of the crosslinked polyamine particles have a particle size of between 500 μm and 1500 μm, a particle size distribution such that no more than 10 vol. % of the crosslinked polyamine particles have a particle size less than 375 μm, a particle size distribution such that the crosslinked polyamine particles have a d₁₀ value that is between 350 μm and 650 a particle size distribution such that the crosslinked polyamine particles have a d₉₀ value that is between 1100 μm and 1400 μm, a particle size distribution such that the .crosslinked polyamine particles have a d₅₀ between 675 μm and 1000 μm, a particle size distribution such that 75 vol. % to 100 vol. % of the crosslinked polyamine particles have a mesh size that is −14/+50, a particle size distribution where greater than 50 vol. % of the crosslinked polyamine particles have a mesh size that is −12/+35, a particle size distribution such that no more than 10 vol. % of the crosslinked polyamine particles have a mesh size that is −45 and/or a particle size distribution such that the crosslinked polyamine particles have an average mesh size of −18/+30, where the crosslinked polyamine particles further comprise a competitive phosphate binding capacity at 60 minutes of greater than 1.2 mmol/g and 500 to 1000 constituent particles, the constituent particles having a d₁₀ value between 20 μm and 70 Rm, a d₉₀ value between 150 μm and 400 μm and/or a d₅₀ of between 70 tim and 120 μm.

In some embodiments, the crosslinked polyamine particles comprise polyallylamine crosslinked with 9.0 wt. % to 10 wt. %, such as 9.5 wt. % to 10 wt. % epichlorohydrin where the crosslinked polyamine particles are in the form of a base and/or a hydrochloride or carbonate salt and have a particle size distribution such that 90 vol. % or greater of the crosslinked polyamine particles have a size between 300 μm and 2000 μm, a particle size distribution where greater than 50 vol. % of the crosslinked polyamine particles have a particle size of between 500 μm and 1500 μm, a particle size distribution such that no more than 10 vol. % of the crosslinked polyamine particles have a particle size less than 375 μm, a particle size distribution such that the crosslinked polyamine particles have a d₁₀ value that is between 350 μm and 650 μm, a particle size distribution such that the crosslinked polyamine particles have a d₉₀ value that is between 1100 μm and 1400 μm, a particle size distribution such that the crosslinked polyamine particles have a d₅₀ between 675 μm and 1000 μm, a particle size distribution such that 75 vol. % to 100 vol. % of the crosslinked polyamine particles have a mesh size that is −14/+50, a particle size distribution where greater than 50 vol. % of the crosslinked polyamine particles have a mesh size that is −12/+35, a particle size distribution such that no more than 10 vol. % of the crosslinked polyamine particles have a mesh size that is −45 and/or a particle size distribution such that the crosslinked polyamine particles have an average mesh size of −18/+30, where the crosslinked polyamine particles further comprise a mean gray value of greater than 190, a competitive phosphate binding capacity at 60 minutes of greater than 1.2 mmol/g and 500 to 1000 constituent particles, the constituent particles having a d₁₀ value between 20 μm and 70 μm, a d₉₀ value between 150 μm and 400 μm and/or a d₅₀ of between 70 μm and 120 μm.

In some embodiments, the crosslinked polyamine particles comprise polyallylamine crosslinked with 9.0 wt. % to 10 wt. %, such as 9.5 wt. % to 10 wt. % epichlorohydrin where the crosslinked polyamine particles are in the form of a base and/or a hydrochloride or carbonate salt and comprise crosslinked polyamine particles having a mean gray value greater than 190, where the crosslinked polyamine particles further comprise 500 to 1000 constituent particles, the constituent particles having a d₁₀ value between 20 μm and 70 μm, a d₉₀ value between 150 μm and 400 μm and/or a d₅₀ of between 70 μm and 120 μm.

In some embodiments, the crosslinked polyamine particles comprise polyallylamine crosslinked with 9.0 wt. % to 10 wt. %, such as 9.5 wt. % to 10 wt. % epichlorohydrin where the crosslinked polyamine particles are in the form of a base and/or a hydrochloride or carbonate salt and comprise crosslinked polyamine particles having a mean gray value greater than 190, where the crosslinked polyamine particles further comprise a competitive phosphate binding capacity at 60 minutes of greater than 1.2 mmol/g.

In some embodiments, the crosslinked polyamine particles comprise polyallylamine crosslinked with 9.0 wt. % to 10 wt. %, such as 9.5 wt. % to 10 wt. % epichlorohydrin where the crosslinked polyamine particles are in the form of a base and/or a hydrochloride or carbonate salt and comprise crosslinked polyamine particles having a mean gray value greater than 190, where the crosslinked polyamine particles further comprise a competitive phosphate binding capacity at 60 minutes of greater than 1.2 mmol/g and 500 to 1000 constituent particles, the constituent particles having a d₁₀ value between 20 μm and 70 μm, a d₉₀ value between 150 μm and 400 μm and/or a d₅₀ of between 70 μm and 120 μm.

In some embodiments, the crosslinked polyamine particles comprise polyallylamine crosslinked with 9.0 wt. % to 10 wt. %, such as 9.5 wt. % to 10 wt. % epichlorohydrin where the crosslinked polyamine particles are in the form of a base and/or a hydrochloride or carbonate salt and comprise crosslinked polyamine particles comprising 500 to 1000 constituent particles, the constituent particles having a d₁₀ value between 20 μm and 70 μm, a d₉₀ value between 150 μm and 400 μm and/or a d₅₀ of between 70 μm and 120 μm and where the crosslinked polyamine particles further comprise a competitive phosphate binding capacity at 60 minutes of greater than 1.2 mmol/g.

In some embodiments, the crosslinked polyamine particles comprise repeat units according to Formula I and/or Formula II, where m is 0 or 1, n is an integer, each R₁, each R₂ and each R₃ are H or a link, are crosslinked with 9.0 wt. % to 10 wt. %, such as 9.5 wt. % to 10 wt. % epichlorohydrin crosslinker, are in the form of a base and/or a hydrochloride or carbonate salt and have a particle size distribution such that 90 vol. % or greater of the crosslinked polyamine particles have a size between 300 μm and 2000 μm, a particle size distribution where greater than 50 vol. % of the crosslinked polyamine particles have a particle size of between 500 μm and 1500 μm, a particle size distribution such that no more than 10 vol. % of the crosslinked polyamine particles have a particle size less than 375 μm, a particle size distribution such that the crosslinked polyamine particles have a d₁₀ value that is between 350 μm and 650 μm, a particle size distribution such that the crosslinked polyamine particles have a d₉₀ value that is between 1100 μm and 1400 μm, a particle size distribution such that the crosslinked polyamine particles have a d₅₀ between 675 μm and 1000 μm, a particle size distribution such that 75 vol. % to 100 vol. % of the crosslinked polyamine particles have a mesh size that is −14/+50, a particle size distribution where greater than 50 vol. % of the crosslinked polyamine particles have a mesh size that is −12/+35, a particle size distribution such that no more than 10 vol. % of the crosslinked polyamine particles have a mesh size that is −45 and/or a particle size distribution such that the crosslinked polyamine particles have an average mesh size of −18/+30.

In some embodiments, the crosslinked polyamine particles comprise repeat units according to Formula I and/or Formula II, where m is 0 or 1, n is an integer, each R₁, each R₂ and each R₃ are H or a link, are crosslinked with 9.0 wt. % to 10 wt. %, such as 9.5 wt. % to 10 wt. % epichlorohydrin crosslinker, are in the form of a base and/or a hydrochloride or carbonate salt and have a mean gray value greater than 190.

In some embodiments, the crosslinked polyamine particles comprise repeat units according to Formula I and/or Formula II, where m is 0 or 1, n is an integer, each R₁, each R₂ and each R₃ are H or a link, are crosslinked with 9.0 wt. % to 10 wt. %, such as 9.5 wt. % to 10 wt. % epichlorohydrin crosslinker, are in the form of a base and/or a hydrochloride or carbonate salt and comprise 500 to 1000 constituent particles, the constituent particles having a d₁₀ value between 20 μm and 70 μm, a d₉₀ value between 150 μm and 400 μm and/or a d₅₀ of between 70 μm and 120 μm.

In some embodiments, the crosslinked polyamine particles comprise repeat units according to Formula I and/or Formula II, where m is 0 or 1, n is an integer, each R₁, each R₂ and each R₃ are H or a link, are crosslinked with 9.0 wt. % to 10 wt. %, such as 9.5 wt. % to 10 wt. % epichlorohydrin crosslinker, are in the form of a base and/or a hydrochloride or carbonate salt and comprise a competitive phosphate binding capacity at 60 minutes of greater than 1.2 mmol/g.

In some embodiments, the crosslinked polyamine particles comprise repeat units according to Formula I and/or Formula II, where m is 0 or 1, n is an integer, each R₁, each R₂ and each R₃ are H or a link, are crosslinked with 9.0 wt. % to 10 wt. %, such as 9.5 wt. % to 10 wt. % epichlorohydrin crosslinker, are in the form of a base and/or a hydrochloride or carbonate salt and have a particle size distribution such that 90 vol. % or greater of the crosslinked polyamine particles have a size between 300 μm and 2000 μm, a particle size distribution where greater than 50 vol. % of the crosslinked polyamine particles have a particle size of between 500 μm and 1500 μm, a particle size distribution such that no more than 10 vol. % of the crosslinked polyamine particles have a particle size less than 375 μm, a particle size distribution such that the crosslinked polyamine particles have a d₁₀ value that is between 350 μm and 650 μm, a particle size distribution such that the crosslinked polyamine particles have a d₉₀ value that is between 1100 μm and 1400 μm, a particle size distribution such that the crosslinked polyamine particles have a d₅₀ between 675 μm and 1000 μm, a particle size distribution such that 75 vol. % to 100 vol. % of the crosslinked polyamine particles have a mesh size that is −14/+50, a particle size distribution where greater than 50 vol. % of the crosslinked polyamine particles have a mesh size that is −12/+35, a particle size distribution such that no more than 10 vol. % of the crosslinked polyamine particles have a mesh size that is −45 and/or a particle size distribution such that the crosslinked polyamine particles have an average mesh size of −18/+30, where the crosslinked polyamine particles further comprise a mean gray value of greater than 190.

In some embodiments, the crosslinked polyamine particles comprise repeat units according to Formula I and/or Formula II, where m is 0 or 1, n is an integer, each R₁, each R₂ and each R₃ are H or a link, are crosslinked with 9.0 wt. % to 10 wt. %, such as 9.5 wt. % to 10 wt. % epichlorohydrin crosslinker, are in the form of a base and/or a hydrochloride or carbonate salt and have a particle size distribution such that 90 vol. % or greater of the crosslinked polyamine particles have a size between 300 μm and 2000 μm, a particle size distribution where greater than 50 vol. % of the crosslinked polyamine particles have a particle size of between 500 μm and 1500 μm, a particle size distribution such that no more than 10 vol. % of the crosslinked polyamine particles have a particle size less than 375 μm, a particle size distribution such that the crosslinked polyamine particles have a d₁₀ value that is between 350 μm and 650 μm, a particle size distribution such that the crosslinked polyamine particles have a d₉₀ value that is between 1100 μm and 400 μm, a particle size distribution such that the crosslinked polyamine particles have a d₅₀ between 675 μm and 1000 μm, a particle size distribution such that 75 vol. % to 100 vol. % of the crosslinked polyamine particles have a mesh size that is −14/+50, a particle size distribution where greater than 50 vol. % of the crosslinked polyamine particles have a mesh size that is −12/+35, a particle size distribution such that no more than 10 vol. % of the crosslinked polyamine particles have a mesh size that is −45 and/or a particle size distribution such that the crosslinked polyamine particles have an average mesh size of −18/+30, where the crosslinked polyamine particles further comprise or are formed from 500 to 1000 constituent particles, the constituent particles having a d₁₀ value between 20 μm and 70 μm, a d₉₀ value between 150 μm and 400 μm and/or a d₅₀ of between 70 μm and 120 μm.

In some embodiments, the crosslinked polyamine particles comprise repeat units according to Formula I and/or Formula II, where m is 0 or 1, n is an integer, each R₁, each R₂ and each R₃ are H or a link, are crosslinked with 9.0 wt. % to 10 wt. %, such as 9.5 wt. % to 10 wt. % epichlorohydrin crosslinker, are in the form of a base and/or a hydrochloride or carbonate salt and have a particle size distribution such that 90 vol. % or greater of the crosslinked polyamine particles have a size between 300 μm and 2000 μm, a particle size distribution where greater than 50 vol. % of the crosslinked polyamine particles have a particle size of between 500 μm and 1500 μm, a particle size distribution such that no more than 10 vol. % of the crosslinked polyamine particles have a particle size less than 375 μm, a particle size distribution such that the crosslinked polyamine particles have a d₁₀ value that is between 350 μm and 650 μm, a particle size distribution such that the crosslinked polyamine particles have a d₉₀ value that is between 1100 μm and 1400 μm, a particle size distribution such that the crosslinked polyamine particles have a d₅₀ between 675 μm and 1000 μm, a particle size distribution such that 75 vol. % to 100 vol. % of the crosslinked polyamine particles have a mesh size that is −14/+50, a particle size distribution where greater than 50 vol. % of the crosslinked polyamine particles have a mesh size that is −12/+35, a particle size distribution such that no more than 10 vol. % of the crosslinked polyamine particles have a mesh size that is −45 and/or a particle size distribution such that the crosslinked polyamine particles have an average mesh size of −18/+30, where the crosslinked polyamine particles further comprise a competitive phosphate binding capacity at 60 minutes of greater than 1.2 mmol/g.

In some embodiments, the crosslinked polyamine particles comprise repeat units according to Formula I and/or Formula II, where m is 0 or 1, n is an integer, each R₁, each R₂ and each R₃ are H or a link, are crosslinked with 9.0 wt. % to 10 wt. %, such as 9.5 wt. % to 10 wt. % epichlorohydrin crosslinker, are in the form of a base and/or a hydrochloride or carbonate salt and have a particle size distribution such that 90 vol. % or greater of the crosslinked polyamine particles have a size between 300 μm and 2000 μm, a particle size distribution where greater than 50 vol. % of the crosslinked polyamine particles have a particle size of between 500 μm and 1500 μm, a particle size distribution such that no more than 10 vol. % of the crosslinked polyamine particles have a particle size less than 375 μm, a particle size distribution such that the crosslinked polyamine particles have a _{d10} value that is between 350 μm and 650 μm, a particle size distribution such that the crosslinked polyamine particles have a d₉₀ value that is between 1100 μm and 1400 μm, a particle size distribution such that the crosslinked polyamine particles have a d₅₀ between 675 μm and 1000 μm, a particle size distribution such that 75 vol. % to 100 vol. % of the crosslinked polyamine particles have a mesh size that is −14/+50, a particle size distribution where greater than 50 vol. % of the crosslinked polyamine particles have a mesh size that is −12/+35, a particle size distribution such that no more than 10 vol. % of the crosslinked polyamine particles have a mesh size that is −45 and/or a particle size distribution such that the crosslinked polyamine particles have an average mesh size of −18/+30, where the crosslinked polyamine particles further comprise a mean gray value of greater than 190 and 500 to 1000 constituent particles, the constituent particles having a d₁₀ value between 20 μm and 70 μm, a d₉₀ value between 150 μm and 400 μm and/or a d₅₀ of between 70 μm and 120 μm.

In some embodiments, the crosslinked polyamine particles comprise repeat units according to Formula I and/or Formula II, where m is 0 or 1, n is an integer, each R₁ each R₂ and each R₃ are H or a link, are crosslinked with 9.0 wt. % to 10 wt. %, such as 9.5 wt. % to 10 wt. % epichlorohydrin crosslinker, are in the form of a base and/or a hydrochloride or carbonate salt and have a particle size distribution such that 90 vol. % or greater of the crosslinked polyamine particles have a size between 300 μm and 2000 μm, a particle size distribution where greater than 50 vol. % of the crosslinked polyamine particles have a particle size of between 500 μm and 1500 μm, a particle size distribution such that no more than 10 vol. % of the crosslinked polyamine particles have a particle size less than 375 μm, a particle size distribution such that the crosslinked polyamine particles have a d₁₀ value that is between 350 μm and 650 μm, a particle size distribution such that the crosslinked polyamine particles have a d₉₀ value that is between 1100 μm and 1400 μm, a particle size distribution such that the crosslinked polyamine particles have a d₅₀ between 675 μm and 1000 μm, a particle size distribution such that 75 vol. % to 100 vol. % of the crosslinked polyamine particles have a mesh size that is −14/+50, a particle size distribution where greater than 50 vol. % of the crosslinked polyamine particles have a mesh size that is −12/+35, a particle size distribution such that no more than 10 vol. % of the crosslinked polyamine particles have a mesh size that is −45 and/or a particle size distribution such that the crosslinked polyamine particles have an average mesh size of −18/+30, where the crosslinked polyamine particles further comprise a mean gray value of greater than 190 and a competitive phosphate binding capacity at 60 minutes of greater than 1.2 mmol/g.

In some embodiments, the crosslinked polyamine particles comprise repeat units according to Formula I and/or Formula II, where m is 0 or 1, n is an integer, each R₁, each R₂ and each R₃ are H or a link, are crosslinked with 9.0 wt. % to 10 wt. %, such as 9.5 wt. % to 10 wt. % epichlorohydrin crosslinker, are in the form of a base and/or a hydrochloride or carbonate salt and have a particle size distribution such that 90 vol. % or greater of the crosslinked polyamine particles have a size between 300 μm and 2000 μm, a particle size distribution where greater than 50 vol. % of the crosslinked polyamine particles have a particle size of between 500 μm and 1500 μm, a particle size distribution such that no more than 10 vol. % of the crosslinked polyamine particles have a particle size less than 375 μm, a particle size distribution such that the crosslinked polyamine particles have a dₗₒ value that is between 350 μm and 650 μm, a particle size distribution such that the crosslinked polyamine particles have a d₉₀ value that is between 1100 μm and 1400 μm, a particle size distribution such that the crosslinked polyamine particles have a d₅₀ between 675 μm and 1000 μm, a particle size distribution such that 75 vol. % to 100 vol. % of the crosslinked polyamine particles have a mesh size that is −14/+50, a particle size distribution where greater than 50 vol. % of the crosslinked polyamine particles have a mesh size that is −12/+35, a particle size distribution such that no more than 10 vol. % of the crosslinked polyamine particles have a mesh size that is −45 and/or a particle size distribution such that the crosslinked polyamine particles have an average mesh size of −18/+30, where the crosslinked polyamine particles further comprise a competitive phosphate binding capacity at 60 minutes of greater than 1.2 mrnol/g and 500 to 1000 constituent particles, the constituent particles having a d₁₀ value between 20 μm and 70 μm, a d₉₀ value between 150 μm and 400 μm and/or a d₅₀ of between 70 μm and 120 μm.

In some embodiments, the crosslinked polyamine particles comprise repeat units according to Formula I and/or Formula II, where m is 0 or 1, n is an integer, each R₁, each R₂ and each R₃ are H or a link, are crosslinked with 9.0 wt. % to 10 wt. %, such as 9.5 wt. % to 10 wt. % epichlorohydrin crosslinker, are in the form of a base and/or a hydrochloride or carbonate salt and have a particle size distribution such that 90 vol. % or greater of the crosslinked polyamine particles have a size between 300 μm and 2000 μm, a particle size distribution where greater than 50 vol. % of the crosslinked polyamine particles have a particle size of between 500 μm and 1500 μm, a particle size distribution such that no more than 10 vol. % of the crosslinked polyamine particles have a particle size less than 375 μm, a particle size distribution such that the crosslinked polyamine particles have a d₁₀ value that is between 350 μm and 650 μm, a particle size distribution such that the crosslinked polyamine particles have a d₉₀ value that is between 1100 μm and 1400 μm, a particle size distribution such that the crosslinked polyamine particles have a d₅₀ between 675 μm and 1000 μm, a particle size distribution such that 75 vol. % to 100 vol. % of the crosslinked polyamine particles have a mesh size that is −14/+50, a particle size distribution where greater than 50 vol. % of the crosslinked polyamine particles have a mesh size that is −12/+35, a particle size distribution such that no more than 10 vol. % of the crosslinked polyamine particles have a mesh size that is −45 and/or a particle size distribution such that the crosslinked polyamine particles have an average mesh size of −18/+30, where the crosslinked polyamine particles further comprise a mean gray value of greater than 190, a competitive phosphate binding capacity at 60 minutes of greater than 1.2 mmol/g and 500 to 1000 constituent particles, the constituent particles having a d₁₀ value between 20 μm and 70 μm, a d₉₀ value between 150 μm and 400 μm and/or a d₅₀ of between 70 μm and 120 μm.

In some embodiments, the crosslinked polyamine particles comprise repeat units according to Formula I and/or Formula II, where m is 0 or 1, n is an integer, each R₁, each R₂ and each R₃ are H or a link, are crosslinked with 9.0 wt. % to 10 wt. %, such as 9.5 wt. % to 10 wt. % epichlorohydrin crosslinker, are in the form of a base and/or a hydrochloride or carbonate salt and have a mean gray value greater than 190, where the crosslinked polyamine particles further comprise 500 to 1000 constituent particles, the constituent particles having a d₁₀ value between 20 μm and 70 μm, a d₉₀ value between 150 μm and 400 μm and/or a d₅₀ of between 70 μm and 120 μm.

In some embodiments, the crosslinked polyamine particles comprise repeat units according to Formula I and/or Formula II, where m is 0 or 1, n is an integer, each R₁, each R₂ and each R₃ are H or a link, are crosslinked with 9.0 wt. % to 10 wt. %, such as 9.5 wt. % to 10 wt. % epichlorohydrin crosslinker, are in the form of a base and/or a hydrochloride or carbonate salt and have a mean gray value greater than 190, where the crosslinked polyamine particles further comprise a competitive phosphate binding capacity at 60 minutes of greater than 1.2 mmol/g.

In some embodiments, the crosslinked polyamine particles comprise repeat units according to Formula I and/or Formula II, where m is 0 or 1, n is an integer, each R₁, each R₂ and each R₃ are H or a link, are crosslinked with 9.0 wt. % to 10 wt. %, such as 9.5 wt. % to 10 wt. % epichlorohydrin crosslinker, are in the form of a base and/or a hydrochloride or carbonate salt and have a mean gray value greater than 190, where the crosslinked polyamine particles further comprise a competitive phosphate binding capacity at 60 minutes of greater than 1.2 mmol/g and 500 to 1000 constituent particles, the constituent particles having a d₁₀ value between 20 μm and 70 μm, a d₉₀ value between 150 μm and 400 μm and/or a d₅₀ of between 70 μm and 120 μm.

In some embodiments, the crosslinked polyamine particles comprise repeat units according to Formula I and/or Formula II, where m is 0 or 1, n is an integer, each R₁, each R₂ and each R₃ are H or a link, are crosslinked with 9.0 wt. % to 10 wt. %, such as 9.5 wt. % to 10 wt. % epichlorohydrin crosslinker, are in the form of a base and/or a hydrochloride or carbonate salt and 500 to 1000 constituent particles, the constituent particles having a d₁₀ value between 20 μm and 70 μm, a d₉₀ value between 150 μm and 400 μm and/or a d₅₀ of between 70 μm and 120 μm and where the crosslinked polyamine particles further comprise a competitive phosphate binding capacity at 60 minutes of greater than 1.2 mmol/g.

In some embodiments, crosslinked polyamine polymers of the invention may be formed using or starting from epichlorohydrin crosslinked polyallylamine carbonate (such as sevelamer carbonate). In some embodiments, epichlorohydrin crosslinked polyallylamine carbonate aggregate particles may be formed by forming constituent particles of sevelamer carbonate having a d₅₀ of between 70 μm and 120 μm, suspending the constituent particles in a solvent such as water, forming a gel from the suspended particles, drying the suspended particles or the gel, optionally milling or grinding the dried particles and fractionating the particles into aggregate particles having a particle size distribution such that 90 vol. % or greater of the crosslinked polyamine particles have a size between 300 μm and 2000 μm, a particle size distribution where greater than 50 vol. % of the crosslinked polyamine particles have a particle size of between 500 μm and 1500 μm, a particle size distribution such that no more than 10 vol. % of the crosslinked polyamine particles have a particle size less than 375 μm, a particle size distribution such that the crosslinked polyamine particles have a d₁₀ value that is between 350 μm and 650 μm, a particle size distribution such that the crosslinked polyamine particles have a d₉₀ value that is between 1100 μm and 1400 μm, a particle size distribution such that the crosslinked polyamine particles have a d₅₀ between 675 μm and 1000 μm, a particle size distribution such that 75 vol. % to 100 vol. % of the crosslinked polyamine particles have a mesh size that is −14/+50, a particle size distribution where greater than 50 vol. % of the . crosslinked polyamine particles have a mesh size that is −12/+35, a particle size distribution such that no more than 10 vol. % of the crosslinked polyamine particles have a mesh size that is −45 and/or a particle size distribution such that the crosslinked polyamine particles have an average mesh size of −18/+30, where the crosslinked polyamine particles further comprise a mean gray value of greater than 190, a competitive phosphate binding capacity at 60 minutes of greater than 1.2 mmol/g and/or 500 to 1000 constituent particles, the constituent particles having a d₁₀ value between 20 μm and 70 μm, a d₉₀ value between 150 μm and 400 μm and/or a d₅₀ of between 70 μm and 120 μm.

In some embodiments, the crosslinked polyamine particles comprise polyallylamine crosslinked with 9.0 wt. % to 10 wt. %, such as 9.5 wt. % to 10 wt. % epichlorohydrin where the crosslinked polyamine particles are in the form of a base and/or a hydrochloride or carbonate salt and have a particle size distribution such that 90 vol. % or greater of the crosslinked polyamine particles have a size between 300 μm and 2000 μm, a particle size distribution where greater than 50 vol. % of the crosslinked polyamine particles have a particle size of between 500 μm and 1500 μm, a particle size distribution such that no more than 10 vol. % of the crosslinked polyamine particles have a particle size less than 375 μm, a particle size distribution such that the crosslinked polyamine particles have a d₁₀ value that is between 350 μm and 650 μm, a particle size distribution such that the crosslinked polyamine particles have a d₉₀ value that is between 1100 μm and 1400 μm, and/or a particle size distribution such that the crosslinked polyamine particles have a d₅₀ between 675 μm and 1000 μm.

In some embodiments, the crosslinked polyamine particles comprise polyallylamine crosslinked with 9.0 wt. % to 10 wt. %, such as 9.5 wt. % to 10 wt. % epichlorohydrin where the crosslinked polyamine particles are in the form of a base and/or a hydrochloride or carbonate salt and have a particle size distribution such that 90 vol. % or greater of the crosslinked polyamine particles have a size between 300 μm and 2000 μm, a particle size distribution where greater than 50 vol. % of the crosslinked polyamine particles have a particle size of between 500 μm and 1500 μm, a particle size distribution such that no more than 10 vol. % of the crosslinked polyamine particles have a particle size less than 375 μm, a particle size distribution such that the crosslinked polyamine particles have a d₁₀ value that is between 350 μm and 650 μm, a particle size distribution such that the crosslinked polyamine particles have a d₉₀ value that is between 1100 μm and 1400 μm, and/or a particle size distribution such that the crosslinked polyamine particles have a d₅₀ between 675 μm and 1000 μm, where the crosslinked polyamine particles further comprise a mean gray value of greater than 190.

In some embodiments, the crosslinked polyamine particles comprise polyallylamine crosslinked with 9.0 wt. % to 10 wt. %, such as 9.5 wt. % to 10 wt. % epichlorohydrin where the crosslinked polyamine particles are in the form of a base and/or a hydrochloride or carbonate salt and have a particle size distribution such that 90 vol. % or greater of the crosslinked polyamine particles have a size between 300 μm and 2000 μm, a particle size distribution where greater than 50 vol. % of the crosslinked polyamine particles have a particle size of between 500 μm and 1500 μm, a particle size distribution such that no more than 10 vol. % of the crosslinked polyamine particles have a particle size less than 375 μm, a particle size distribution such that the crosslinked polyamine particles have a d₁₀ value that is between 350 μm and 650 μm, a particle size distribution such that the crosslinked polyamine particles have a d₉₀ value that is between 1100 μm and 1400 μm, and/or a particle size distribution such that the crosslinked polyamine particles have a d₅₀ between 675 μm and 1000 μm, where the crosslinked polyamine particles further comprise or are formed from 500 to 1000 constituent particles, the constituent particles having a d₁₀ value between 20 μm and 70 μm, a d₉₀ value between 150 μm and 400 μm and/or a d₅₀ of between 70 μm and 120 μm.

In some embodiments, the crosslinked polyamine particles comprise polyallylamine crosslinked with 9.0 wt. % to 10 wt. %, such as 9.5 wt. % to 10 wt. % epichlorohydrin where the crosslinked polyamine particles are in the form of a base and/or a hydrochloride or carbonate salt and have a particle size distribution such that 90 vol. % or greater of the crosslinked polyamine particles have a size between 300 μm and 2000 μm, a particle size distribution where greater than 50 vol. % of the crosslinked polyamine particles have a particle size of between 500 μm and 1500 μm, a particle size distribution such that no more than 10 vol. % of the crosslinked polyamine particles have a particle size less than 375 μm, a particle size distribution such that the crosslinked polyamine particles have a d₁₀ value that is between 350 μm and 650 μm, a particle size distribution such that the crosslinked polyamine particles have a d₉₀ value that is between 1100 μm and 1400 μm, and/or a particle size distribution such that the crosslinked polyamine particles have a d₅₀ between 675 μm and 1000 μm, where the crosslinked polyamine particles further comprise a competitive phosphate binding capacity at 60 minutes of greater than 1.2 mmol/g.

In some embodiments, the crosslinked polyamine particles comprise polyallylamine crosslinked with 9.0 wt. % to 10 wt. %, such as 9.5 wt. % to 10 wt. % epichlorohydrin where the crosslinked polyamine particles are in the form of a base and/or a hydrochloride or carbonate salt and have a particle size distribution such that 90 vol. % or greater of the crosslinked polyamine particles have a size between 300 μm and 2000 μm, a particle size distribution where greater than 50 vol. % of the crosslinked polyamine particles have a particle size of between 500 μm and 1500 μm, a particle size distribution such that no more than 10 vol. % of the crosslinked polyamine particles have a particle size less than 375 μm, a particle size distribution such that the crosslinked polyamine particles have a d₁₀ value that is between 350 μm and 650 μm, a particle size distribution such that the crosslinked polyamine particles have a d₉₀ value that is between 1100 μm and 1400 μm, and/or a particle size distribution such that the crosslinked polyamine particles have a d₅₀ between 675 μm and 1000 where the crosslinked polyamine particles further comprise a mean gray value of greater than 190 and 500 to 1000 constituent particles, the constituent particles having a d₁₀ value between 20 μm and 70 μm, a d₉₀ value between 150 μm and 400 μm and/or a d₅₀ of between 70 μm and 120 μm.

In some embodiments, the crosslinked polyamine particles comprise polyallylamine crosslinked with 9.0 wt. % to 10 wt. %, such as 9.5 wt. % to 10 wt. % epichlorohydrin where the crosslinked polyamine particles are in the form of a base and/or a hydrochloride or carbonate salt and have a particle size distribution such that 90 vol. % or greater of the crosslinked polyamine particles have a size between 300 μm and 2000 μm, a particle size distribution where greater than 50 vol. % of the crosslinked polyamine particles have a particle size of between 500 μm and 1500 μm, a particle size distribution such that no more than 10 vol. % of the crosslinked polyamine particles have a particle size less than 375 μm, a particle size distribution such that the crosslinked polyamine particles have a d₁₀ value that is between 350 μm and 650 μm, a particle size distribution such that the crosslinked polyamine particles have a d₉₀ value that is between 1100 μm and 1400 μm, and/or a particle size distribution such that the crosslinked polyamine particles have a d₅₀ between 675 μm and 1000 μm, where the crosslinked polyamine particles further comprise a mean gray value of greater than 190 and a competitive phosphate binding capacity at 60 minutes of greater than 1.2 mmol/g.

In some embodiments, the crosslinked polyamine particles comprise polyallylamine crosslinked with 9.0 wt. % to 10 wt. %, such as 9.5 wt. % to 10 wt. % epichlorohydrin where the crosslinked polyamine particles are in the form of a base and/or a hydrochloride or carbonate salt and have a particle size distribution such that 90 vol. % or greater of the crosslinked polyamine particles have a size between 300 μm and 2000 μm, a particle size distribution where greater than 50 vol. % of the crosslinked polyamine particles have a particle size of between 500 μm and 1500 μm, a particle size distribution such that no more than 10 vol. % of the crosslinked polyamine particles have a particle size less than 375 μm, a particle size distribution such that the crosslinked polyamine particles have a d₁₀ value that is between 350 μm and 650 μm, a particle size distribution such that the crosslinked polyamine particles have a d₉₀ value that is between 1100 μm and 1400 μm, and/or a particle size distribution such that the crosslinked polyamine particles have a d₅₀ between 675 μm and 1000 μm, where the crosslinked polyamine particles further comprise a competitive phosphate binding capacity at 60 minutes of greater than 1.2 mmol/g and 500 to 1000 constituent particles, the constituent particles having a d₁₀ value between 20 μm and 70 μm, a d₉₀ value between 150 μm and 400 μm and/or a d₅₀ of between 70 μm and 120 μm.

In some embodiments, the crosslinked polyamine particles comprise polyallylamine crosslinked with 9.0 wt. % to 10 wt. %, such as 9.5 wt. % to 10 wt. % epichlorohydrin where the crosslinked polyamine particles are in the form of a base and/or a hydrochloride or carbonate salt and have a particle size distribution such that 90 vol. % or greater of the crosslinked polyamine particles have a size between 300 μm and 2000 μm, a particle size distribution where greater than 50 vol. % of the crosslinked polyamine particles have a particle size of between 500 μm and 1500 μm, a particle size distribution such that no more than 10 vol. % of the crosslinked polyamine particles have a particle size less than 375 μm, a particle size distribution such that the crosslinked polyamine particles have a d₁₀ value that is between 350 μm and 650 μm, a particle size distribution such that the crosslinked polyamine particles have a d₉₀ value that is between 1100 μm and 1400 μm, and/or a particle size distribution such that the crosslinked polyamine particles have a d₅₀ between 675 μm and 1000 μm, where the crosslinked polyamine particles further comprise a mean gray value of greater than 190, a competitive phosphate binding capacity at 60 minutes of greater than 1.2 mmol/g and 500 to 1000 constituent particles, the constituent particles having a d₁₀ value between 20 μm and 70 μm, a d₉₀ value between 150 μm and 400 μm and/or a d₅₀ of between 70 μm and 120 μm.

In some embodiments, the crosslinked polyamine particles comprise polymers according to Formula III or Formula IV and have a particle size distribution such that 90 vol. % or greater of the crosslinked polyamine particles have a size between 300 μm and 2000 μm, a particle size distribution where greater than 50 vol. % of the crosslinked polyamine particles have a particle size of between 500 μm and 1500 μm, a particle size distribution such that no more than 10 vol. % of the crosslinked polyamine particles have a particle size less than 375 μm, a particle size distribution such that the crosslinked polyamine particles have a d₁₀ value that is between 350 μm and 650 μm, a particle size distribution such that the crosslinked polyamine particles have a d₉₀ value that is between 1100 μm and 1400 μm, a particle size distribution such that the crosslinked polyamine particles have a d₅₀ between 675 μm and 1000 μm, a particle size distribution such that 75 vol. % to 100 vol. % of the crosslinked polyamine particles have a mesh size that is −14/+50, a particle size distribution where greater than 50 vol. % of the crosslinked polyamine particles have a mesh size that is −12/+35, a particle size distribution such that no more than 10 vol. % of the crosslinked polyamine particles have a mesh size that is −45 and/or a particle size distribution such that the crosslinked polyamine particles have an average mesh size of −18/+30.

In some embodiments, the crosslinked polyamine particles comprise polymers according to Formula III or Formula IV and have a mean gray value greater than 190.

In some embodiments, the crosslinked polyamine particles comprise polymers according to Formula III or Formula IV and comprise 500 to 1000 constituent particles, the constituent particles having a d₁₀ value between 20 μm and 70 μm, a d₉₀ value between 150 μm and 400 μm and/or a d₅₀ of between 70 μm and 120 μm.

In some embodiments, the crosslinked polyamine particles comprise polymers according to Formula III or Formula IV and comprise a competitive phosphate binding capacity at 60 minutes of greater than 1.2 mmol/g.

In some embodiments, the crosslinked polyamine particles comprise polymers according to Formula III or Formula IV and have a particle size distribution such that 90 vol. % or greater of the crosslinked polyamine particles have a size between 300 μm and 2000 μm, a particle size distribution where greater than 50 vol. % of the crosslinked polyamine particles have a particle size of between 500 μm and 1500 m, a particle size distribution such that no more than 10 vol. % of the crosslinked polyamine particles have a particle size less than 375 μm, a particle size distribution such that the crosslinked polyamine particles have a d₁₀ value that is between 350 μm and 650 μm, a particle size distribution such that the crosslinked polyamine particles have a d₉₀ value that is between 1100 μm and 1400 μm, a particle size distribution such that the crosslinked polyamine particles have a d₅₀ between 675 μm and 1000 μm, a particle size distribution such that 75 vol. % to 100 vol. % of the crosslinked polyamine particles have a mesh size that is −14/+50, a particle size distribution where greater than 50 vol. % of the crosslinked polyamine particles have a mesh size that is −12/+35, a particle size distribution such that no more than 10 vol. % of the crosslinked polyamine particles have a mesh size that is −45 and/or a particle size distribution such that the crosslinked polyamine particles have an average mesh size of −18/+30, where the crosslinked polyamine particles further comprise a mean gray value of greater than 190.

In some embodiments, the crosslinked polyamine particles comprise polymers according to Formula III or Formula IV and have a particle size distribution such that 90 vol. % or greater of the crosslinked polyamine particles have a size between 300 μm and 2000 μm, a particle size distribution where greater than 50 vol. % of the crosslinked polyamine particles have a particle size of between 500 μm and 1500 μm, a particle size distribution such that no more than 10 vol. % of the crosslinked polyamine particles have a particle size less than 375 μm, a particle size distribution such that the crosslinked polyamine particles have a d₁₀ value that is between 350 μm and 650 μm, a particle size distribution such that the crosslinked polyamine particles have a d₉₀ value that is between 1100 μm and 1400 μm, a particle size distribution such that the crosslinked polyamine particles have a d₅₀ between 675 μm and 1000 μm, a particle size distribution such that 75 vol. % to 100 vol. % of the crosslinked polyamine particles have a mesh size that is −14/+50, a particle size distribution where greater than 50 vol. % of the crosslinked polyamine particles have a mesh size that is −12/+35, a particle size distribution such that no more than 10 vol. % of the crosslinked polyamine particles have a mesh size that is −45 and/or a particle size distribution such that the crosslinked polyamine particles have an average mesh size of −18/+30, where the crosslinked polyamine particles further comprise or are formed from 500 to 1000 constituent particles, the constituent particles having a d₁₀ value between 20 μm and 70 μm, a d₉₀ value between 150 μm and 400 μm and/or a d₅₀ of between 70 μm and 120 μm.

In some embodiments, the crosslinked polyamine particles comprise polymers according to Formula III or Formula IV and have a particle size distribution such that 90 vol. % or greater of the crosslinked polyamine particles have a size between 300 μm and 2000 μm, a particle size distribution where greater than 50 vol. % of the crosslinked polyamine particles have a particle size of between 500 μm and 1500 μm, a particle size distribution such that no more than 10 vol. % of the crosslinked polyamine particles have a particle size less than 375 μm, a particle size distribution such that the crosslinked polyamine particles have a d₁₀ value that is between 350 μm and 650 μm, a particle size distribution such that the crosslinked polyamine particles have a d₉₀ value that is between 1100 μm and 1400 μm, a particle size distribution such that the crosslinked polyamine particles have a d₅₀ between 675 μm and 1000 μm, a particle size distribution such that 75 vol. % to 100 vol. % of the crosslinked polyamine particles have a mesh size that is −14/+50, a particle size distribution where greater than 50 vol. % of the crosslinked polyamine particles have a mesh size that is −12/+35, a particle size distribution such that no more than 10 vol. % of the crosslinked polyamine particles have a mesh size that is −45 and/or a particle size distribution such that the crosslinked polyamine particles have an average mesh size of −18/+30, where the crosslinked polyamine particles further comprise a competitive phosphate binding capacity at 60 minutes of greater than 1.2 mmol/g.

In some embodiments, the crosslinked polyamine particles comprise polymers according to Formula III or Formula IV and have a particle size distribution such that 90 vol. % or greater of the crosslinked polyamine particles have a size between 300 μm and 2000 μm, a particle size distribution where greater than 50 vol. % of the crosslinked polyamine particles have a particle size of between 500 μm and 1500 μm, a particle size distribution such that no more than 10 vol. % of the crosslinked polyamine particles have a particle size less than 375 μm, a particle size distribution such that the crosslinked polyamine particles have a d₁₀ value that is between 350 μm and 650 μm, a particle size distribution such that the crosslinked polyamine particles have a d₉₀ value that is between 1100 μm and 1400 μm, a particle size distribution such that the crosslinked polyamine particles have a d₅₀ between 675 μm and 1000 μm, a particle size distribution such that 75 vol. % to 100 vol. % of the crosslinked polyamine particles have a mesh size that is −14/+50, a particle size distribution where greater than 50 vol. % of the crosslinked polyamine particles have a mesh size that is −12/+35, a particle size distribution such that no more than 10 vol. % of the crosslinked polyamine particles have a mesh size that is −45 and/or a particle size distribution such that the crosslinked polyamine particles have an average mesh size of −18/+30, where the crosslinked polyamine particles further comprise a mean gray value of greater than 190 and 500 to 1000 constituent particles, the constituent particles having a d₁₀ value between 20 μm and 70 μm, a d₉₀ value between 150 μm and 400 μm and/or a d₅₀ of between 70 μm and 120 μm.

In some embodiments, the crosslinked polyamine particles comprise polymers according to Formula III or Formula IV and have a particle size distribution such that 90 vol. % or greater of the crosslinked polyamine particles have a size between 300 μm and 2000 μm, a particle size distribution where greater than 50 vol. % of the crosslinked polyamine particles have a particle size of between 500 μm and 1500 μm, a particle size distribution such that no more than 10 vol. % of the crosslinked polyamine particles have a particle size less than 375 μm, a particle size distribution such that the crosslinked polyamine particles have a d₁₀ value that is between 350 μm and 650 μm, a particle size distribution such that the crosslinked polyamine particles have a d₉₀ value that is between 1100 μm and 1400 μm, a particle size distribution such that the crosslinked polyamine particles have a d₅₀ between 675 μm and 1000 μm, a particle size distribution such that 75 vol. % to 100 vol. % of the crosslinked polyamine particles have a mesh size that is −14/+50, a particle size distribution where greater than 50 vol. % of the crosslinked polyamine particles have a mesh size that is −12/+35, a particle size distribution such that no more than 10 vol. % of the crosslinked polyamine particles have a mesh size that is −45 and/or a particle size distribution such that the crosslinked polyamine particles have an average mesh size of −18/+30, where the crosslinked polyamine particles further comprise a mean gray value of greater than 190 and a competitive phosphate binding capacity at 60 minutes of greater than 1.2 mmol/g.

In some embodiments, the crosslinked polyamine particles comprise polymers according to Formula III or Formula IV and have a particle size distribution such that 90 vol. % or greater of the crosslinked polyamine particles have a size between 300 μm and 2000 μm, a particle size distribution where greater than 50 vol. % of the crosslinked polyamine particles have a particle size of between 500 μm and 1500 μm, a particle size distribution such that no more than 10 vol. %of the crosslinked polyamine particles have a particle size less than 375 μm, a particle size distribution such that the crosslinked polyamine particles have a d₁₀ value that is between 350 μm and 650 μm, a particle size distribution such that the crosslinked polyamine particles have a d₉₀ value that is between 1100 μm and 1400 μm, a particle size distribution such that the crosslinked polyamine particles have a d₅₀ between 675 μm and 1000 μm, a particle size distribution such that 75 vol. % to 100 vol. % of the crosslinked polyamine particles have a mesh size that is −14/+50, a particle size distribution where greater than 50 vol. % of the crosslinked polyamine particles have a mesh size that is −12/+35, a particle size distribution such that no more than 10 vol. % of the crosslinked polyamine particles have a mesh size that is −45 and/or a particle size distribution such that the crosslinked polyamine particles have an average mesh size of −18/+30, where the crosslinked polyamine particles further comprise a competitive phosphate binding capacity at 60 minutes of greater than 1.2 mmol/g and 500 to 1000 constituent particles, the constituent particles having a d₁₀ value between 20 μm and 70 μm, a d₉₀ value between 150 μm and 400 μm and/or a d₅₀ of between 70 μm and 120 μm.

In some embodiments, the crosslinked polyamine particles comprise polymers according to Formula III or Formula IV and have a particle size distribution such that 90 vol. % or greater of the crosslinked polyamine particles have a size between 300 μm and 2000 μm, a particle size distribution where greater than 50 vol. % of the crosslinked polyamine particles have a particle size of between 500 μm and 1500 μm, a particle size distribution such that no more than 10 vol. % of the crosslinked polyamine particles have a particle size less than 375 μm, a particle size distribution such that the crosslinked polyamine particles have a d₁₀ value that is between 350 μm and 650 μm, a particle size distribution such that the crosslinked polyamine particles have a d₉₀ value that is between 1100 μm and 1400 μm, a particle size distribution such that the crosslinked polyamine particles have a d₅₀ between 675 μm and 1000 μm, a particle size distribution such that 75 vol. % to 100 vol. % of the crosslinked polyamine particles have a mesh size that is −14/+50, a particle size distribution where greater than 50 vol. % of the crosslinked polyamine particles have a mesh size that is −12/+35, a particle size distribution such that no more than 10 vol. % of the crosslinked polyamine particles have a mesh size that is −45 and/or a particle size distribution such that the crosslinked polyamine particles have an average mesh size of −18/+30, where the crosslinked polyamine particles further comprise a mean gray value of greater than 190, a competitive phosphate binding capacity at 60 minutes of greater than 1.2 mmol/g and 500 to 1000 constituent particles, the constituent particles having a d₁₀ value between 20 μm and 70 μm, a d₉₀ value between 150 μm and 400 μm and/or a d₅₀ of between 70 μm and 120 μm.

In some embodiments, the crosslinked polyamine particles comprise polymers according to Formula III or Formula IV and have a mean gray value greater than 190, where the crosslinked polyamine particles further comprise 500 to 1000 constituent particles, the constituent particles having a d₁₀ value between 20 μm and 70 μm, a d₉₀ value between 150 μm and 400 μm and/or a d₅₀ of between 70 μm and 120 μm.

In some embodiments, the crosslinked polyamine particles comprise polymers according to Formula III or Formula IV and have a mean gray value greater than 190, where the crosslinked polyamine particles further comprise a competitive phosphate binding capacity at 60 minutes of greater than 1.2 mmol/g.

In some embodiments, the crosslinked polyamine particles comprise polymers according to Formula III or Formula IV and have a mean gray value greater than 190, where the crosslinked polyamine particles further comprise a competitive phosphate binding capacity at 60 minutes of greater than 1.2 mmol/g and 500 to 1000 constituent particles, the constituent particles having a d₁₀ value between 20 μm and 70 μm, a d₉₀ value between 150 μm and 400 μm and/or a d₅₀ of between 70 μm and 120 μm.

In some embodiments, the crosslinked polyamine particles comprise polymers according to Formula III or Formula IV where the crosslinked polyamine particles further comprise 500 to 1000 constituent particles, the constituent particles having a d₁₀ value between 20 μm and 70 μm, a d₉₀ value between 150 μm and 400 μm and/or a d₅₀ of between 70 μm and 120 μm and a competitive phosphate binding capacity at 60 minutes of greater than 1.2 mmol/g.

In some embodiments, the crosslinked polyamine particles comprise polyallylamine crosslinked with 9.0 wt. % to 10 wt. %, such as 9.5 wt. % to 10 wt. % epichlorohydrin where the crosslinked polyamine particles are in the form of a base and/or a hydrochloride or carbonate salt and have a particle size distribution such that 90 vol. % or greater of the crosslinked polyamine particles have a size between 300 μm and 2000 μm, a particle size distribution such that the crosslinked polyamine particles have a d₁₀ value that is between 350 μm and 650 μm, a particle size distribution such that the crosslinked amine particles have a d₉₀ value that is between 1100 μm and 1400 μm, a particle size distribution such that the crosslinked polyamine particles have a d₅₀ between 675 μm and 1000 μm.

In some embodiments, the crosslinked polyamine particles comprise polyallylamine crosslinked with 9.0 wt. % to 10 wt. %, such as 9.5 wt. % to 10 wt. % epichlorohydrin where the crosslinked polyamine particles are in the form of a base and/or a hydrochloride or carbonate salt and have a particle size distribution such that 90 vol. % or greater of the crosslinked polyamine particles have a size between 300 μm and 2000 μm, a particle size distribution such that the crosslinked polyamine particles have a d₁₀ value that is between 350 μm and 650 μm, a particle size distribution such that the crosslinked amine particles have a d₉₀ value that is between 1100 μm and 1400 μm, a particle size distribution such that the crosslinked polyamine particles have a d₅₀ between 675 μm and 1000 μm, where the crosslinked polyamine particles further comprise a mean gray value of greater than 190.

In some embodiments, the crosslinked polyamine particles comprise polyallylamine crosslinked with 9.0 wt. % to 10 wt. %, such as 9.5 wt. % to 10 wt. % epichlorohydrin where the crosslinked polyamine particles are in the form of a base and/or a hydrochloride or carbonate salt and have a particle size distribution such that 90 vol. % or greater of the crosslinked polyamine particles have a size between 300 μm and 2000 μm, a particle size distribution such that the crosslinked polyamine particles have a d₁₀ value that is between 350 μm and 650 μm, a particle size distribution such that the crosslinked amine particles have a d₉₀ value that is between 1100 μm and 1400 μm, a particle size distribution such that the crosslinked polyamine particles have a d₅₀ between 675 μm and 1000 μm, where the crosslinked polyamine particles further comprise or are formed from 500 to 1000 constituent particles, the constituent particles having a d₁₀ value between 20 μm and 70 μm, a d₉₀ value between 150 μm and 400 μm and/or a d₅₀ of between 70 μm and 120 μm.

In some embodiments, the crosslinked polyamine particles comprise polyallylamine crosslinked with 9.0 wt. % to 10 wt. %, such as 9.5 wt. % to 10 wt. % epichlorohydrin where the crosslinked polyamine particles are in the form of a base and/or a hydrochloride or carbonate salt and have a particle size distribution such that 90 vol. % or greater of the crosslinked polyamine particles have a size between 300 μm and 2000 μm, a particle size distribution such that the crosslinked polyamine particles have a d₁₀ value that is between 350 μm and 650 μm, a particle size distribution such that the crosslinked amine particles have a d₉₀ value that is between 1100 μm and 1400 μm, a particle size distribution such that the crosslinked polyamine particles have a d₅₀ between 675 μm and 1000 μm, where the crosslinked polyamine particles further comprise a competitive phosphate binding capacity at 60 minutes of greater than 1.2 mmol/g.

In some embodiments, the crosslinked polyamine particles comprise polyallylamine crosslinked with 9.0 wt. % to 10 wt. %, such as 9.5 wt. % to 10 wt. % epichlorohydrin where the crosslinked polyamine particles are in the form of a base and/or a hydrochloride or carbonate salt and have a particle size distribution such that 90 vol. % or greater of the crosslinked polyamine particles have a size between 300 μm and 2000 μm, a particle size distribution such that the crosslinked polyamine particles have a D₁₀ value that is between 350 μm and 650 μm, a particle size distribution such that the crosslinked amine particles have a d₉₀ value that is between 1100 μm and 1400 μm, a particle size distribution such that the crosslinked polyamine particles have a d₅₀ between 675 μm and 1000 μm, where the crosslinked polyamine particles further comprise a mean gray value of greater than 190 and 500 to 1000 constituent particles, the constituent particles having a d₁₀ value between 20 μm and 70 μm, a d₉₀ value between 150 μm and 400 μm and/or a d50 of between 70 μm and 120 μm.

In some embodiments, the crosslinked polyamine particles comprise polyallylamine crosslinked with 9.0 wt. % to 10 wt. %, such as 9.5 wt. % to 10 wt. % epichlorohydrin where the crosslinked polyamine particles are in the form of a base and/or a hydrochloride or carbonate salt and have a particle size distribution such that 90 vol. % or greater of the crosslinked polyamine particles have a size between 300 μm and 2000 μm, a particle size distribution such that the crosslinked polyamine particles have a d₁₀ value that is between 350 μm and 650 μm, a particle size distribution such that the crosslinked amine particles have a d₉₀ value that is between 1100 μm and 1400 μm, a particle size distribution such that the crosslinked polyamine particles have a d₅₀ between 675 μm and 1000 μm, where the crosslinked polyamine particles further comprise a mean gray value of greater than 190 and a competitive phosphate binding capacity at 60 minutes of greater than 1.2 mmol/g.

In some embodiments, the crosslinked polyamine particles comprise polyallylamine crosslinked with 9.0 wt. % to 10 wt. %, such as 9.5 wt. % to 10 wt. % epichlorohydrin where the crosslinked polyamine particles are in the form of a base and/or a hydrochloride or carbonate salt and have a particle size distribution such that 90 vol. % or greater of the crosslinked polyamine particles have a size between 300 μm and 2000 μm, a particle size distribution such that the crosslinked polyamine particles have a d₁₀ value that is between 350 μm and 650 μm, a particle size distribution such that the crosslinked amine particles have a d₉₀ value that is between 1100 μm and 1400 μm, a particle size distribution such that the cirosslinked polyamine particles have a d₅₀ between 675 μm and 1000 μm, where the crosslinked polyamine particles further comprise a competitive phosphate binding capacity at 60 minutes of greater than 1.2 mmol/g and 500 to 1000 constituent particles, the constituent particles having a d₁₀ value between 20 μm and 70 μm, a d₉₀ value between 150 μm and 400 μm and/or a d₅₀ of between 70 μm and 120 μm.

In some embodiments, the crosslinked polyamine particles comprise polyallylamine crosslinked with 9.0 wt. % to 10 wt. %, such as 9.5 wt. % to 10 wt. % epichlorohydrin where the crosslinked polyamine particles are in the form of a base and/or a hydrochloride or carbonate salt and have a particle size distribution such that 90 vol. % or greater of the crosslinked polyamine particles have a size between 300 μm and 2000 μm, a particle size distribution such that the crosslinked polyamine particles have a d₁₀ value that is between 350 μm and 650 μm, a particle size distribution such that the crosslinked amine particles have a d₉₀ value that is between 1100 μm and 1400 μm, a particle size distribution such that the crosslinked polyamine particles have a d₅₀ between 675 μm and 1000 μm, where the crosslinked polyamine particles further comprise a mean gray value of greater than 190, a competitive phosphate binding capacity at 60 minutes of greater than 1.2 mmol/g and 500 to 1000 constituent particles, the constituent particles having a d₁₀ value between 20 μm and 70 μm, a d₉₀ value between 150 μm and 400 μm and/or a d₅₀ of between 70 μm and 120 μm.

In some embodiments, the crosslinked polyamine particles comprise repeat units according to Formula I and/or Formula II, where m is 0 or 1, n is an integer, each R₁, each R₂ and each R₃ are H or a link, are crosslinked with 9.0 wt. % to 10 wt. %, such as 9.5 wt. % to 10 wt. % epichlorohydrin crosslinker, are in the form of a base and/or a hydrochloride or carbonate salt and have a particle size distribution such that 90 vol. % or greater of the crosslinked polyamine particles have a size between 300 μm and 2000 μm, a particle size distribution such that the crosslinked polyamine particles have a d₁₀ value that is between 350 μm and 650 μm, a particle size distribution such that the crosslinked amine particles have a d₉₀ value that is between 1100 μm and 1400 μm, a particle size distribution such that the crosslinked polyamine particles have a d₅₀ between 675 μm and 1000 μm.

In some embodiments, the crosslinked polyamine particles comprise repeat units according to Formula I and/or Formula II, where m is 0 or 1, n is an integer, each R₁, each R₂ and each R₃ are H or a link, are crosslinked with 9.0 wt. % to 10 wt. %, such as 9.5 wt. % to 10 wt. % epichlorohydrin crosslinker, are in the form of a base and/or a hydrochloride or carbonate salt and have a particle size distribution such that 90 vol. % or greater of the crosslinked polyamine particles have a size between 300 μm and 2000 μm, a particle size distribution such that the crosslinked polyamine particles have a d₁₀ value that is between 350 μm and 650 μm, a particle size distribution such that the crosslinked amine particles have a d₉₀ value that is between 1100 μm and 1400 μm, a particle size distribution such that the crosslinked polyamine particles have a d₅₀ between 675 μm and 1000 where the crosslinked polyamine particles further comprise a mean gray value of greater than 190.

In some embodiments, the crosslinked polyamine particles comprise repeat units according to Formula I and/or Formula II, where m is 0 or 1, n is an integer, each R₁, each R₂ and each R₃ are H or a link, are crosslinked with 9.0 wt. % to 10 wt. %, such as 9.5 wt. % to 10 wt. % epichlorohydrin crosslinker, are in the form of a base and/or a hydrochloride or carbonate salt and have a particle size distribution such that 90 vol. % or greater of the crosslinked polyamine particles have a size between 300 μm and 2000 μm, a particle size distribution such that the crosslinked polyamine particles have a d₁₀ value that is between 350 μm and 650 μm, a particle size distribution such that the crosslinked amine particles have a d₉₀ value that is between 1100 μm and 1400 μm, a particle size distribution such that the crosslinked polyamine particles have a d₅₀ between 675 μm and 1000 μm, where the crosslinked polyamine particles further comprise or are formed from 500 to 1000 constituent particles, the constituent particles having a d₁₀ value between 20 μm and 70 μm, a d₉₀ value between 150 μm and 400 μm and/or a d₅₀ of between 70 μm and 120 μm.

In some embodiments, the crosslinked polyamine particles comprise repeat units according to Formula I and/or Formula II, where m is 0 or 1, n is an integer, each R₁, each R₂ and each R₃ are H or a link, are crosslinked with 9.0 wt. % to 10 wt. %, such as 9.5 wt. % to 10 wt. % epichlorohydrin crosslinker, are in the form of a base and/or a hydrochloride or carbonate salt and have a particle size distribution such that 90 vol. % or greater of the crosslinked polyamine particles have a size between 300 μm and 2000 μm, a particle size distribution such that the crosslinked polyamine particles have a d₁₀ value that is between 350 μm and 650 μm, a particle size distribution such that the crosslinked amine particles have a d₉₀ value that is between 1100 μm and 1400 μm, a particle size distribution such that the crosslinked polyamine particles have a d₅₀ between 675 μm and 1000 μm, where the crosslinked polyamine particles further comprise a competitive phosphate binding capacity at 60 minutes of greater than 1.2 mmol/g.

In some embodiments, the crosslinked polyamine particles comprise repeat units according to Formula I and/or Formula II, where m is 0 or 1, n is an integer, each R₁, each R₂ and each R₃ are H or a link, are crosslinked with 9.0 wt. % to 10 wt. %, such as 9.5 wt. % to 10 wt. % epichlorohydrin crosslinker, are in the form of a base and/or a hydrochloride or carbonate salt and have a particle size distribution such that 90 vol. % or greater of the crosslinked polyamine particles have a size between 300 μm and 2000 μm, a particle size distribution such that the crosslinked polyamine particles have a d₁₀ value that is between 350 μm and 650 μm, a particle size distribution such that the crosslinked amine particles have a d₉₀ value that is between 1100 μm and 1400 μm, a particle size distribution such that the crosslinked polyamine particles have a d₅₀ between 675 μm and 1000 μm, where the crosslinked polyamine particles further comprise a mean gray value of greater than 190 and 500 to 1000 constituent particles, the constituent particles having a d₁₀ value between 20 μm and 70 μm, a d₉₀ value between 150 μm and 400 μm and/or a d₅₀ of between 70 μm and 120 μm.

In some embodiments, the crosslinked polyamine particles comprise repeat units according to Formula I and/or Formula II, where m is 0 or 1, n is an integer, each R₁, each R₂ and each R₃ are H or a link, are crosslinked with 9.0 wt. % to 10 wt. %, such as 9.5 wt. % to 10 wt. % epichlorohydrin crosslinker, are in the form of a base and/or a hydrochloride or carbonate salt and have a particle size distribution such that 90 vol. % or greater of the crosslinked polyamine particles have a size between 300 μm and 2000 μm, a particle size distribution such that the crosslinked polyamine particles have a d₁₀ value that is between 350 μm and 650 μm, a particle size distribution such that the crosslinked amine particles have a d₉₀ value that is between 1100 μm and 1400 μm, a particle size distribution such that the crosslinked polyamine particles have a d₅₀ between 675 μm and 1000 μm, where the crosslinked polyamine particles further comprise a mean gray value of greater than 190 and a competitive phosphate binding capacity at 60 minutes of greater than 1.2 mmol/g.

In some embodiments, the crosslinked polyamine particles comprise repeat units according to Formula I and/or Formula II, where m is 0 or 1, n is an integer, each R₁, each R₂ and each R₃ are H or a link, are crosslinked with 9.0 wt. % to 10 wt. %, such as 9.5 wt. % to 10 wt. % epichlorohydrin crosslinker, are in the form of a base and/or a hydrochloride or carbonate salt and have a particle size distribution such that 90 vol. % or greater of the crosslinked polyamine particles have a size between 300 μm and 2000 μm, a particle size distribution such that the crosslinked polyamine particles have a d₁₀ value that is between 350 μm and 650 μm, a particle size distribution such that the crosslinked amine particles have a d₉₀ value that is between 1100 μm and 1400 μm, a particle size distribution such that the crosslinked polyamine particles have a d₅₀ between 675 μm and 1000 μm, where the crosslinked polyamine particles further comprise a competitive phosphate binding capacity at 60 minutes of greater than 1.2 mmol/g and 500 to 1000 constituent particles, the constituent particles having a d₁₀ value between 20 μm and 70 μm, a d₉₀ value between 150 μm and 400 μm and/or a d₅₀ of between 70 μm and 120 μm.

In some embodiments, the crosslinked polyamine particles comprise repeat units according to Formula I and/or Formula II, where m is 0 or 1, n is an integer, each R₁, each R₂ and each R₃ are H or a link, are crosslinked with 9.0 wt. % to 10 wt. %, such as 9.5 wt. % to 10 wt. % epichlorohydrin crosslinker, are in the form of a base and/or a hydrochloride or carbonate salt and have a particle size distribution such that 90 vol. % or greater of the crosslinked polyamine particles have a size between 300 μm and 2000 μm, a particle size distribution such that the crosslinked polyamine particles have a d₁₀ value that is between 350 μm and 650 μm, a particle size distribution such that the crosslinked amine particles have a d₉₀ value that is between 1100 μm and 1400 μm, a particle size distribution such that the crosslinked polyamine particles have a d₅₀ between 675 μm and 1000 μm, where the crosslinked polyamine particles further comprise a mean gray value of greater than 190, a competitive phosphate binding capacity at 60 minutes of greater than 1.2 mmol/g and 500 to 1000 constituent particles, the constituent particles having a d₁₀ value between 20 μm and 70 μm, a d₉₀ value between 150 μm and 400 μm and/or a d₅₀ of between 70 μm and 120 μm.

In some embodiments, the crosslinked polyamine particles comprise repeat units according to Formula I and/or Formula II, where m is 0 or 1, n is an integer, each R₁, each R₂ and each R₃ are H or a link, are crosslinked with 9.0 wt. % to 10 wt. %, such as 9.5 wt. % to 10 wt. % epichlorohydrin crosslinker, are in the form of a base and/or a hydrochloride or carbonate salt and have an acid stability; as measured by comparing the particle sizes after treatment in acid, such as 1N HCl, of cured particles (for example, cured in 60° C. for 3 weeks or about 110° C. for 4 hours, after drying), with acid treated crosslinked polyamine particles that have not been cured; of greater than 1.5 fold, such as greater than 1.7 fold the particle size of acid treated crosslinked polyamine particles that have not been cured.

In some embodiments, the crosslinked polyamine particles comprise repeat units according to Formula I and/or Formula II, where m is 0 or 1, n is an integer, each R₁, each R₂ and each R₃ are H or a link, are crosslinked with 9.0 wt. % to 10 wt. %, such as 9.5 wt. % to 10 wt. % epichlorohydrin crosslinker, are in the form of a base and/or a hydrochloride or carbonate salt and have an acid stability; as measured by comparing the particle sizes after treatment in acid, such as 1N HCl, of cured particles (for example, cured in 60° C. for 3 weeks or about 110° C. for 4 hours, after drying), with acid treated crosslinked polyamine particles that have not been cured; of greater than 1.5 fold, such as greater than 1.7 fold the particle size of acid treated crosslinked polyamine particles that have not been cured and a Volume Weighted Mean of the cured particle, in accordance with the Wet Particle Size & Distribution (in Acid) test method, of between 300 and 450 μm.

In some embodiments, the crosslinked polyamine particles comprise repeat units according to Formula I and/or Formula II, where m is 0 or 1, n is an integer, each R₁, each R₂ and each R₃ are H or a link, are crosslinked with 9.0 wt. % to 10 wt. %, such as 9.5 wt. % to 10 wt. % epichlorohydrin crosslinker, are in the form of a base and/or a hydrochloride or carbonate salt and have an acid stability; as measured by comparing the particle sizes after treatment in acid, such as 1N HCl, of cured particles (for example, cured in 60° C. for 3 weeks or about 110° C. for 4 hours, after drying), with acid treated crosslinked polyamine particles that have not been cured; of greater than 1.5 fold, such as greater than 1.7 fold the particle size of acid treated crosslinked polyamine particles that have not been cured and a Volume Weighted Mean of the cured particle, in accordance with the Determination of Dry Particle Size & Distribution test, of between 750 and 900 μm.

In some embodiments, the dry sevelamer carbonate particles may be rehydrated to a % LOD of excess of 35, for example between 40 and 50, optionally co-milled and then placed in a dryer, for example a forced air oven, at a temperature between 100° C. to 120° C. for 3 to 5 hours (or a % LOD of between 20 to 35) optional in a nitrogen purge, with periodic agitation. The material is then co-milled with a screen between 1450 μm and 1650 μm. The material is then futher dried to a % LOD of less than 5% LOD, for example less than 2.5% LOD. The particles may be sieved to the desired or specified size, for example particles having a d₁₀ value greater than 500 μm, for example between 520 μm and 600μm, a d₉₀ of greater than 1000, for example between 1200 μm and 1500 μm and/or a d₅₀ of greater than 700, for example between 750 μm and 1000 μm; and then may be optional further cured. In some embodiments, the sieved particles may be cured in a forced air oven at a temperature of between 90° C. to 120° C. for about 3 to 6 hours.

In some embodiments, the sevelamer carbonate gel particles having a % LOD of greater than 50% LOD resulting from the preparation process may be sieved to a % LOD of between 20 and 35% LOD, for example 28 to 32% LOD, and co-milled with a screen between 1450 μm and 1650 μm. The material may then be dried in a dryer, for example a forced air oven, at a temperature between 100° C. to 120° C. for 3 to 5 hours (or a % LOD of between 20 to 35) optional in a nitrogen purge, with periodic agitation. The material is then co-milled with a screen between 1450 μm and 1650 μm. The material is then futher dried to a % LOD of less than 5% LOD, for example less than 2.5% LOD. The particles may be sieved to the desired or specified size, for example particles having a d₁₀ value greater than 500 μm, for example between 520 μm and 600μm, a d₉₀ of greater than 1000, for example between 1200 μm and 1500 μm and/or a d₅₀ of greater than 700, for example between 750 μm and 1000 μm, and then may be optional further cured. In some embodiments, the sieved particles may be cured in a forced air oven at a temperature of between 90° C. to 120° C. for about 3 to 6 hours.

In some embodiments, the dry sevelamer carbonate particles may be rehydrated to a % LOD of excess of 35, for example between 40 and 50, optionally co-milled and then placed in a dryer, for example a forced air oven, at a temperature between 100° C. to 120° C. for 3 to 5 hours (or a % LOD of between 20 to 35) optional in a nitrogen purge, with periodic agitation. The material is then co-milled with a screen between 1450 μm and 1650 μm. The material is then futher dried to a % LOD of less than 5% LOD, for example less than 2.5% LOD. The particles may be sieved to the desired or specified size, for example, particles having a d₁₀ value greater than 500 μm, for example between 520 μm and 600μm, a d₉₀ of greater than 1000, for example between 1200 μm and 1500 μm and/or a d₅₀ of greater than 700, for example between 750 μm and 1000 μm; and then may be optional further cured. In some embodiments, the sieved particles may be cured in a forced air oven at a temperature of between 90° C. to 120° C. for about 3 to 6 hours.

In some embodiments, the dry sevelamer carbonate particles may be rehydrated to a % LOD of excess of 35, for example between 40 and 50, optionally co-milled and then placed in a dryer, for example a forced air oven, at a temperature between 100° C. to 120° C. for 3 to 5 hours (or a % LOD of between 20 to 35) optional in a nitrogen purge, with periodic agitation. The material is then co-milled with a screen between 1450 μm and 1650 μm. The material is then futher dried to a % LOD of less than 5% LOD, for example less than 2.5% LOD. The particles may be sieved to the desired or specified size, for example particles having a d₁₀ value greater than 500 μm, for example between 520 μm and 600 μm, a d₉₀ of greater than 1000, for example between 1200 μm and 1500 μm and/or a d₅₀ of greater than 700, for example between 750 μm and 1000 μm; and then may be optional further cured. In some embodiments, the sieved particles may be cured in a forced air oven at a temperature of between 90° C. to 120° C. for about 3 to 6 hours.

The cured particles may have volume weighted mean particle size, when measured in accordance with the Wet Particle Size & Distribution (in Acid) test method, of greater than 350 μm, for example greater than 375 μm, greater than 400 μm, greater than 425 μm, greater than 450 μm, greater than 475 μm, or between 425 μm and 750 μm. The particles may additional or alternatively have a volume weighted mean particle size, when measured in accordance with the Wet Particle Size & Distribution (in Phosphate Buffer) test method of greater than 500 μm, for example greater than 525 μm, greater than 550 μm, greater than 575 μm, greater than 600 μm, greater than 625 μm, greater than 650 μm, greater than 675 μm, greater than 700 μm , or between 600 μm and 800 μm.

In some embodiments, the crosslinked polyamine particles comprise repeat units according to Formula I and/or Formula II, where m is 0 or 1, n is an integer, each R₁, each R₂ and each R₃ are H or a link, are crosslinked with 9.0 wt. % to 10 wt. %, such as 9.5 wt. % to 10 wt. % epichlorohydrin crosslinker, are in the form of a base and/or a hydrochloride or carbonate salt. The constituent particles have a dry particle size distribution of a d₁₀ value between 20 μm and 70 μm, a d₉₀ value between 150 μm and 400 μm and/or a d₅₀ of between 70 μm and 120 μm. In some embodiments, the dry sevelamer carbonate particles may be rehydrated to a % LOD of excess of 35, for example between 40 and 50, optionally co-milled and then placed in a dryer, for example a forced air oven, at a temperature between 100° C. to 120° C. for 3 to 5 hours (or a % LOD of between 20 to 35) optional in a nitrogen purge, with periodic agitation. The material is then co-milled with a screen between 1450 μm and 1650 μm. The material is then futher dried to a % LOD of less than 5% LOD, for example less than 2% LOD. The particles may be sieved to the desired or specified size, for example particles having a d₁₀ value greater than 500 μm, for example between 520 μm and 600μm, a d₉₀ of greater than 1000 μm, for example between 1200 μm and 1500 μm and/or a d₅₀ of greater than 700, for example between 750 μm and 1000 μm; and then may be optional further cured. In some embodiments, the sieved particles may be cured in a forced air oven at a temperature of between 90° C. to 120° C. for about 3 to 6 hours. These particles can then be tabletted into a core tablet comprising greater than 70 wt. %, for example between 75 wt. % and 85 wt. % of the tablet. The tablet may be optionally coated. The coated tablet comprising greater than 750 mg, for example between 775 and 825 mg with particles having a dry particle, volume weighted mean of 400 μm and 1200 μm, for example greater than 500 μm.

In some embodiments, coated tablets comprising between 775 and 825 mg of crosslinked polyamine are formed from cured crosslinked polyamine particles comprising repeat units according to Formula I and/or Formula II, where m is 0 or 1, n is an integer, each R₁, each R₂ and each R₃ are H or a link, are crosslinked with 9.0 wt. % to 10 wt. %, such as 9.5 wt. % to 10 wt. % epichlorohydrin crosslinker, are in the form of a base and/or a hydrochloride or carbonate salt, wherein the particles having a dry particle, volume weighted mean of 400 μm and 1200 μm, for example greater than 500 μm. The constituent particles have a dry particle size distribution of a d₁₀ value between 20 μm and 70 μm, a d₉₀ value between 150 μm and 400 μm and/or a d₅₀ of between 70 μm and 120 μm. In some embodiments, the dry sevelamer carbonate particles may be rehydrated to a % LOD of excess of 35, for example between 40 and 50, optionally co-milled and then placed in a dryer, for example a forced air oven, at a temperature between 100° C. to 120° C. for 3 to 5 hours (or a % LOD of between 20 to 35) optional in a nitrogen purge, with periodic agitation. The material is then co-milled with a screen between 1450 μm and 1650 μm. The material is then father dried to a % LOD of less than 5% LOD, for example less than 2% LOD. The particles may be sieved to the desired or specified size, for example particles having a dᵢₒ value greater than 500 μm, for example between 520 μm and 600μm, a d₉₀ of greater than 1000 μm, for example between 1200 μm and 1500 μm and/or a d₅₀ of greater than 700, for example between 750 μm and 1000 μm; and then may be optional further cured. In some embodiments, the sieved particles may be cured in a forced air oven at a temperature of between 90° C. to 120° C. for about 3 to 6 hours. These cured particles can then be tabletted into a core tablet comprising greater than 70 wt. %, for example between 75 wt. % and 85 wt. % of the tablet. The tablet is then coated. The coated tablet, upon dissolution in a phosphate buffer in accordance with the Tablet Dissolution Particle Size & Distribution test method, has a Volume Weighted Mean of between 400 μm and 625 μm.

In some embodiments, coated tablets comprising between 775 and 825 mg of crosslinked polyamine are formed from cured crosslinked polyamine particles comprising repeat units according to Formula I and/or Formula II, where m is 0 or 1, n is an integer, each R₁, each R₂ and each R₃ are H or a link, are crosslinked with 9.0 wt. % to 10 wt. %, such as 9.5 wt. % to 10 wt. % epichlorohydrin crosslinker, are in the form of a base and/or a hydrochloride or carbonate salt, wherein the particles having a dry particle, volume weighted mean greater than 575 μm. These cured particles can then be tabletted into a core tablet comprising greater than 70 wt. %, for example between 75 wt. % and 85 wt. % of the tablet. The tablet is then coated. The coated tablet, upon dissolution in a phosphate buffer in accordance with the Tablet Dissolution Particle Size & Distribution test method, has a Volume Weighted Mean of between 430 μm and 575 μm.

In some embodiments, coated tablets comprising between 775 and 825 mg of crosslinked polyamine are formed from cured crosslinked polyamine particles comprising repeat units according to Formula I and/or Formula II, where m is 0 or 1, n is an integer, each R₁, each R₂ and each R₃ are H or a link, are crosslinked with 9.0 wt. % to 10 wt. %, such as 9.5 wt. % to 10 wt. % epichlorohydrin crosslinker, are in the form of a base and/or a hydrochloride or carbonate salt, wherein the particles having a dry particle, volume weighted mean greater than 575 μm. These cured particles can then be tabletted into a core tablet comprising greater than 70 wt. %, for example between 75 wt. % and 85 wt. % of the tablet. The tablet is then coated. The coated tablet, upon dissolution in a 1N HCl, in accordance with the Tablet Dissolution Particle Size & Distribution (in Acid) test method, has a Volume Weighted Mean of between 325 μm and 550 μm.

In some embodiments, coated tablets comprising between 775 and 825 mg of crosslinked polyamine are formed from cured crosslinked polyamine particles comprising repeat units according to Formula I and/or Formula II, where m is 0 or 1, n is an integer, each R₁, each R₂ and each R₃ are H or a link, are crosslinked with 9.0 wt. % to 10 wt. %, such as 9.5 wt. % to 10 wt. % epichlorohydrin crosslinker, are in the form of a base and/or a hydrochloride or carbonate salt. These cured particles can then be tabletted into a core tablet comprising greater than 70 wt. %, for example between 75 wt. % and 85 wt. % of the tablet. The tablet is then coated. The coated tablet, upon dissolution in a 1N HCl in accordance with the Tablet Dissolution Particle Size & Distribution (in Acid) test method, has a Volume Weighted Mean of between 325 μm and 550 μm and, upon dissolution in a phosphate buffer in accordance with the Tablet Dissolution Particle Size & Distribution (in Phosphate Buffer) test method, has a Volume Weighted Mean of between 430 μm and 575 μm.

In some embodiments, coated tablets comprising between 775 and 825 mg of crosslinked polyamine are formed from cured crosslinked polyamine particles comprising repeat units according to Formula I and/or Formula II, where m is 0 or 1, n is an integer, each R₁, each R₂ and each R₃ are H or a link, are crosslinked with 9.0 wt. % to 10 wt. %, such as 9.5 wt. % to 10 wt. % epichlorohydrin crosslinker, are in the form of a base and/or a hydrochloride or carbonate salt, wherein the particles having a dry particle, volume weighted mean greater than 575 μm. These cured particles can then be tabletted into a core tablet comprising greater than 70 wt. %, for example between 75 wt. % and 85 wt. % of the tablet. The tablet is then coated. The coated tablet, upon dissolution in a IN HCl, in accordance with the Tablet Dissolution Particle Size & Distribution (in Acid) test method, has a Volume Weighted Mean of between 325 μm and 550 μm and, upon dissolution in a phosphate buffer in accordance with the Tablet Dissolution Particle Size & Distribution (in Phosphate Buffer) test method, has a Volume Weighted Mean of between 430 μm and 575 μm and a Volume % Mode of between 475 and 625.

In some embodiments, coated tablets comprising between 775 and 825 mg of crosslinked polyamine are formed from cured crosslinked polyamine particles comprising repeat units according to Formula I and/or Formula II, where m is 0 or 1, n is an integer, each R₁, each R₂ and each R₃ are H or a link, are crosslinked with 9.0 wt. % to 10 wt. %, such as 9.5 wt. % to 10 wt. % epichlorohydrin crosslinker, are in the form of a base and/or a hydrochloride or carbonate salt, wherein the particles having a dry particle, volume weighted mean greater than 575 μm. These cured particles can then be tabletted into a core tablet comprising greater than 70 wt. %, for example between 75 wt. % and 85 wt. % of the tablet. The tablet is then coated. The coated tablet, upon dissolution in a phosphate buffer in accordance with the Tablet Dissolution Particle Size & Distribution test method, has a Volume % Mode of between 475 and 625.

In some embodiments, coated tablets comprising between 775 and 825 mg of crosslinked polyamine are formed from cured crosslinked polyamine particles comprising repeat units according to Formula I and/or Formula II, where m is 0 or 1, n is an integer, each R₁, each R₂ and each R₃ are H or a link, are crosslinked with 9.0 wt. % to 10 wt. %, such as 9.5 wt. % to 10 wt. % epichlorohydrin crosslinker, are in the form of a base and/or a hydrochloride or carbonate salt, wherein the particles having a dry particle, volume weighted mean greater than 575 μm. These cured particles can then be tabletted into a core tablet comprising greater than 70 wt. %, for example between 75 wt. % and 85 wt. % of the tablet. The tablet is then coated. The coated tablet, upon dissolution in a phosphate buffer in accordance with the Tablet Dissolution Particle Size & Distribution test method, has a Volume Weighted Mean of between 430 μm and 575 μm and a Volume % Mode of between 475 and 625.

In some embodiments, coated tablets comprising between 775 and 825 mg of crosslinked polyamine are formed from cured crosslinked polyamine particles comprising repeat units according to Formula I and/or Formula II, where m is 0 or 1, n is an integer, each R₁, each R₂ and each R₃ are H or a link, are crosslinked with 9.0 wt. % to 10 wt. %, such as 9.5 wt. % to 10 wt. % epichlorohydrin crosslinker, are in the form of a base and/or a hydrochloride or carbonate salt. In some embodiments, the cured particles may have a allylamine ppm value of between 0.4 and 1.0, for example between 0.5 and .85, such as 0.6 and 0.75. In some embodiments, the cured particles may have a % soluble oligomers of less than 1.0%, for example less than 0.5%, such as less than 0.1%, or 0.05%; a True Density of between 1.0 and 2 g/cubic centimeter, for example between 1.0 and 1.5 g/cubic centimeter; a Tap Density of between 0.25 and 1 g/ml, for example between 0.4 and 0.6 g/ml; a Bulk Density of between 0.20 and 0.8 g/ml, for example between 0.2 and 0.4 g/ml; a pH of between 9 and 10, for example between 9.3 and 9.7; a DSC-Glass Transition temperature of between 50° C. and 65° C., for example between 55° C. and 60° C.; and/or a competitive phosphate binding of between 1.70 mmol/g and 3.2 mmol/g, for example 1.80 mmol/g and 3.0 mmol/g or 1.90 mmol/g and 2.7 mmol/g or 1.95 mmol/g and 2.5 mmol/g or 1.98 mmol/g and 2.4 mmol/g or 2.0 mmol/g and 2.3 mmol/g. These cured particles can then be tabletted into a core tablet comprising greater than 70 wt. %, for example between 75 wt. % and 85 wt. % of the tablet. The tablet is then coated. The coated tablet, upon dissolution in a 1N HCl in accordance with the Tablet Dissolution Particle Size & Distribution (in Acid) test method, has a Volume Weighted Mean of between 325 μm and 550 μm and, upon dissolution in a phosphate buffer, in accordance with the Tablet Dissolution Particle Size & Distribution (in Phosphate Buffer) test method, has a Volume Weighted Mean of between 430 μm and 575 μm.

One aspect of the invention is core-shell compositions comprising a polymeric core and shell. In some embodiments, the polymeric core comprises the crosslinked polyamine particles described herein. The shell material can be chemically anchored to the core material or physically coated. In the former case, the shell can be grown on the core component through chemical means, for example by: chemical grafting of shell polymer to the core using living polymerization from active sites anchored onto the core polymer; interfacial reaction, i.e., a chemical reaction located at the core particle surface, such as interfacial polycondensation; and using block copolymers as suspending agents during the core particle synthesis.

In some embodiments, the interfacial reaction and use of block polymers are the techniques used when chemical methods are used. In the interfacial reaction pathway, typically, the periphery of the core material is chemically modified by reacting small molecules or macromolecules on the core interface. For example, a crosslinked polyamine core is reacted with a polymer containing amine reactive groups such as epoxy, isocyanate, activated esters or halide groups to form a crosslinked shell around the core.

When the shell material is physically adsorbed on the core material, well known techniques of microencapsulation such as solvent coacervation, fluidized bed spray coater, or multiemulsion processes can be used. One method of microencapsulation is the fluidized bed spray coater in the Wurster configuration. In yet another embodiment, the shell material is only acting temporarily by delaying the swelling of the core while in the mouth and esophagus, and optionally disintegrates in the stomach or duodenum. The shell may be selected in order to hinder the transport of water into the core, by creating a layer of high hydrophobicity and very low liquid water permeability.

In some embodiments, shell materials are polymers carrying negative charges in the pH range typically found in the intestine. Examples include, but are not limited to, polymers that have pendant acid groups such as carboxylic, sulfonic, hydrosulfonic, sulfamic, phosphoric, hydrophosphoric, phosphonic, hydrophosphonic, phosphoramidic, phenolic, boronic and a combination thereof. The polymer can be protonated or unprotonated; in the latter case the acidic anion can be neutralized with pharmaceutically acceptable cations such as Na, K, Li, Ca, Mg, and NH₄.

The shell polymers can be either linear, branched, hyperbranched, segmented (i.e., backbone polymer arranged in sequence of contiguous blocks of which at least one contains pendant acidic groups), comb-shaped, star-shaped or crosslinked in a network, fully and semi-interpenetrated network (IPN). The shell polymers are either random or blocky in composition and either covalently or physically attached to the core material. Examples of such shell polymers include, but are not limited to acrylic acid homopolymers or copolymers, methacrylic acid homopolymers or copolymers, and copolymers of methacrylate and methacrylic acid. Examples of such polymers are copolymers of methyl methacrylate and methacrylic acid and copolymers of ethyl acrylate and methacrylic acid, sold under the tradename Eudragit (Rohm GmbH & Co. KG): examples of which include Eudragit L100-55 and Eudragit L100 (a methyl methacrylate-methacrylic acid (1:1) copolymer, Degussa/Rohm), Eudragit L30-D55, Eudragit S 100-55 and Eudragit FS 30D, Eudragit S 100 (a methyl methacrylate-methacrylic acid (2:1) copolymer), Eudragit LD-55 (an ethyl acrylate-methacrylic acid (1:1) copolymer), copolymers of acrylates and methacrylates with quaternary ammonium groups, sold under the tradenames Eudragit RL and Eudragit RS, and a neutral ester dispersion without any functional groups, sold under the tradename Eudragit NE30-D.

Additional shell polymers include: poly(styrene sulfonate), polyacrylic acid(s); carboxymethyl cellulose, cellulose acetate phthalate, hydroxypropyl methylcellulose phthalate as sold under the tradename HP-50 and HP-55 (Shin-Etsu Chemical Co., Ltd.), cellulose acetate trimellitate, cellulose acetate, cellulose acetate butyrate, cellulose acetate propionate, ethyl cellulose, cellulose derivatives, such as hydroxypropylmethylcellulose, methylcelluose, hydroxylethylcellulose, hydroxyethylmethylcellulose, hydroxylethylethylcelluose and hydroxypropylethylcellulose and cellulose derivatives such as cellulose ethers useful in film coating formulations, polyvinyl acetate phthalate, carrageenan, alginate, or poly(methacrylic acid) esters, acrylic/maleic acid copolymers, styrene/maleic acid polymers, itaconic acid/acrylic copolymers, and fumaric/acrylic acid copolymers, polyvinyl acetal diethylaminoacetate, as sold under the tradename AEA (Sankyo Co., Ltd.), methylvinylether/maleic acid copolymers and shellac.

In some embodiments, the shell polymers are selected amongst pharmaceutically acceptable polymers such as Eudragit L100-55 and Eudragit L100 (a methylmethacrylate-methacrylic acid (1:1) copolymer, Degussa/Rohm), Carbopol 934 (polyacrylic acid, Noveon), C-A-P NF (cellulose acetate phthalate—Eastman), Eastacryl (methacrylic acid esters—Eastman), Carrageenan and Alginate (FMC Biopolymer), Anycoat—P (Samsung Fine Chemicals—HPMC Phthalate), or Aqualon (carboxymethyl cellulose—Hercules), methylvinylether/maleic acid copolymers (Gantrez), and styrene/maleic acid (SMA).

The shell can be coated by a variety of methods. In one embodiment, the shell materials are added in the drug formulation step as an active excipient; for example, the shell material can be included in a solid formulation as a powder, which is physically blended with the crosslinked polyamine and other excipients, optionally granulated, and compressed to form a tablet. Thus, in some embodiments, the shell material need not cover the core material in the drug product. For example, the acidic shell polymer may be added together with the core formulated in the shape of a tablet, capsule, gel, liquid, wafer, extrudates, etc., and the shell polymer can then dissolve and distribute itself uniformly as a shell coating around the core while the drug product equilibrates in the mouth, esophagus or ultimately in the site of action, i.e., the gastrointestinal tract.

In some embodiments, the shell is a thin layer of shell polymer. The layer can be a molecular layer of polyanion on the core material surface. The weight to core ratio can be between about 0.0001% to about 30%, preferably comprised between about 0.01% to about 5%, such as between about 0.1% to about 5%.

The shell polymers have a minimum molecular weight such that they do not freely permeate within the_{.}.core pore volume nor elute from the core surface. In some embodiments, the molecular weight (Mw) of the shell acidic polymer is above about 1000 g/mole, such as above about 5000 g/mole, and or even above about 20,000 g/mole.

The anionic charge density of the shell material (as prevailing in the milieu of use) may be between 0.5 mEq/g to 22 mEq/g, such as 2 mEq/g to 15 mEq/g. If a coating process is used to form the shell on the crosslinked polyamine particles as part of the manufacture of the dosage form, then procedures known from those skilled-in-the-art in the pharmaceutical industry are applicable. In one embodiment, the shell is formed in a fluidized bed coater (Wurster coater). In an alternate embodiment, the shell is formed through controlled precipitation or coascervation, wherein the crosslinked amine polymer particles are suspended in a polymer solution, and the solvent properties are changed in such a way as to induce the polymer to precipitate onto or coat the crosslinked amine polymer particles.

Suitable coating processes include the procedures typically used in the pharmaceutical industry. Typically, selection of the coating method is dictated by a number of parameters that include, but are not limited to, the form of the shell material (bulk, solution, emulsion, suspension, melt) as well as the shape and nature of the core material (spherical beads, irregular shaped, etc.), and the amount of shell deposited. In addition, the cores may be coated with one or more shells and may comprise multiple or alternating layers of shells.

The term “phosphate imbalance disorder” as used herein refers to conditions in which the level of phosphorus present in the body is abnormal. One example of a phosphate imbalance disorder includes hyperphosphatemia. The term “hyperphosphatemia” as used herein refers to a condition in which the element phosphorus is present in the body at an elevated level. Typically, a patient is often diagnosed with hyperphosphatemia if the blood phosphate level is, for example, above about 4.0 or 4.5 milligrams per deciliter of blood, for example above about 5.0 mg/dl, such as above about 5.5 mg/dl, for example above 6.0 mg/dl, and/or the patient has a severely impaired glomerular filtration rate such as, for example, less than about 20% of normal. The present invention may also be used to treat patients suffering from hyperphosphatemia in End Stage Renal Disease and who are also receiving dialysis treatment (e.g., hemodialysis or peritoneal dialysis). Also, the present invention can be used to treat Chronic Kidney Disease (CKD), to treat patients with CKD who are on dialysis and dialysis patients, including prophylactic treatment of any of the above.

Other diseases that can be treated with the methods, polymers, crosslinked polyamine particles, compositions and kits of the present invention include hypocalcemia, hyperparathyroidism, depressed renal synthesis of calcitriol, tetany due to hypocalcemia, renal insufficiency, and ectopic calcification in soft tissues including calcifications in joints, lungs, kidney, conjuctiva, and myocardial tissues including prophylactic treatment of any of the above.

The crosslinked polyamine particles and compositions described herein can be used as an adjunct to other therapies, e.g., those employing dietary control of phosphorus intake, dialysis, inorganic metal salts and/or other polymer resins.

The compositions of the present invention are also useful in removing chloride, bicarbonate, oxalate, and bile acids from the gastrointestinal tract. Crosslinked polyamine particles removing oxalate compounds or ions find use in the treatment of oxalate imbalance disorders, such as oxalosis or hyperoxaluria that increases the risk of kidney stone formation. Crosslinked polyamine particles removing chloride compounds or ions find use in treating acidosis, heartburn, acid reflux disease, sour stomach or gastritis, for example. In some embodiments, the compositions of the present invention are useful for removing fatty acids, bilirubin, and related compounds. Some embodiments may also bind and remove high molecular weight molecules like proteins, nucleic acids, vitamins or cell debris.

The present invention provides methods, pharmaceutical compositions, and kits for the treatment of animals. The term “animal” or “animal subject” or “patient” as used herein includes humans as well as other mammals (e.g., in veterinary treatments, such as in the treatment of dogs or cats, or livestock animals such as pigs, goats, cows and horses) and other livestock animals such as chickens and the like. One embodiment of the invention is a method of removing phosphorous-containing compounds such as organophosphates or phosphate from the gastrointestinal tract, such as the stomach, small intestine or large intestine of an animal by administering an effective amount of the crosslinked polyamine particles described herein.

The term “treating” and its grammatical equivalents as used herein include achieving a therapeutic benefit and/or a prophylactic benefit. By therapeutic benefit is meant eradication, amelioration, or prevention of the underlying disorder being treated. For example, in a hyperphosphatemia patient, therapeutic benefit includes eradication or amelioration of the underlying hyperphosphatemia. Also, a therapeutic benefit is achieved with the eradication, amelioration, or prevention of one or more of the physiological symptoms associated with the underlying disorder such that an improvement is observed in the patient, notwithstanding that the patient may still be afflicted with the underlying disorder. For example, administration of crosslinked polyamine particles, described herein, to a patient suffering from renal insufficiency and/or hyperphosphatemia provides therapeutic benefit not only when the patient's serum phosphate level is decreased, but also when an improvement is observed in the patient with respect to other disorders that accompany renal failure and/or hyperphosphatemia like ectopic calcification and renal osteodistrophy. For prophylactic benefit, for example, the crosslinked polyamine particles may be administered to a patient at risk of developing hyperphosphatemia or to a patient reporting one or more of the physiological symptoms of hyperphosphatemia, even though a diagnosis of hyperphosphatemia may not have been made.

The compositions may also be used to control serum phosphate in subjects with elevated phosphate levels, for example, by changing the serum level of phosphate towards a normal or near normal level, for example, towards a level that is within 10% of the normal level of a healthy patient.

Other embodiments of the invention are directed towards pharmaceutical compositions comprising at least one of the crosslinked polyamine particles or a pharmaceutically acceptable salt of the crosslinked polyamine particles, and one or more pharmaceutically acceptable excipients, diluents, or carriers and optionally additional therapeutic agents. The compositions may be lyophilized or dried under vacuum or oven before formulating.

The excipients or carriers are “acceptable” in the sense of being compatible with the other ingredients of the formulation and not deleterious to the recipient thereof. The formulations can conveniently be presented in unit dosage form and can be prepared by any suitable method. The methods typically include the step of bringing into association the agent with the excipients or carriers such as by uniformly and intimately bringing into association the crosslinked amine polymer with the excipients or carriers and then, if necessary, dividing the product into unit dosages thereof.

The pharmaceutical compositions of the present invention include compositions wherein the crosslinked polyamine particles are present in an effective amount, i.e., in an amount effective to achieve therapeutic and/or prophylactic benefit. The actual amount effective for a particular application will depend on the patient (e.g., age, weight, etc.), the condition being treated, and the route of administration.

The dosages of the crosslinked polyamine particles in animals will depend on the disease being, treated, the route of administration, and the physical characteristics of the animal being treated. Such dosage levels in some embodiments for either therapeutic and/or prophylactic uses may be from about 1 gm/day to about 30 gm/day, for example from about 2 gm/day to about 20 gm/day, from about 2 gm/day to about 10 gm/day, from about 3 gm/day to about 9 gm/day, from about 3 gm/day to about 8 gm/day, from about 3 gm/day to about 7 gm/day, from about 3 gm/day to about 6 gm/day, from about 3 gm/day to about 5 gm/day, from about 4 gm/day to about 7 gm/day or from about 4 gm/day to about 6 gm/day. The dose of the crosslinked amine polymers described herein can be less than about 50 gm/day, less than about 40 gm/day, less than about 30 gm/day, less than about 20 gm/day, and less than about 10 gm/day.

Typically, the crosslinked polyamine particles can be administered before or after a meal, or with a meal. As used herein, “before” or “after” a meal is typically within two hours, preferably within one hour, more preferably within thirty minutes, most preferably within ten minutes of commencing or finishing a meal, respectively.

Generally, it is preferred that the crosslinked polyamine particles are administered along with meals. In some embodiments, the crosslinked polyamine particles may be administered one time a day, two times a day, or three times a day. In some embodiments, the crosslinked polyamine particles are administered once a day with the largest meal.

Preferably, the crosslinked polyamine particles may be used for therapeutic and/or prophylactic benefits and can be administered alone or in the form of a pharmaceutical composition. The pharmaceutical compositions comprise the crosslinked polyamine particles, one or more pharmaceutically acceptable carriers, diluents or excipients, and optionally additional therapeutic agents. For example, the crosslinked polyamine particles of the present invention may be co-administered with other active pharmaceutical agents depending on the condition being treated. Examples of pharmaceutical agents that may be co-administered include, but are not limited to:

Other phosphate sequestrants including pharmaceutically acceptable lanthanum, calcium, aluminum, magnesium, iron and zinc compounds, such as acetates, carbonates, oxides, hydroxides, citrates, alginates, and ketoacids thereof.

Calcium compounds, including calcium carbonate, acetate (such as PhosLo® calcium acetate tablets), citrate, alginate, and ketoacids;

Aluminium-based phosphate sequestrants, such as Amphojel® aluminium hydroxide gel;

Lanthanide compounds such as lanthanum carbonate)(Fosreno®).

Other phosphate sequestrants suitable for use in the present invention include pharmaceutically acceptable magnesium compounds. Various examples of pharmaceutically acceptable magnesium compounds are described in U.S. Provisional Application No. 60/734,593 filed Nov. 8, 2005, the entire teachings of which are incorporated herein by reference. Specific suitable examples include magnesium oxide, magnesium hydroxide, magnesium halides (e.g., magnesium fluoride, magnesium chloride, magnesium bromide and magnesium iodide), magnesium alkoxides (e.g., magnesium ethoxide and magnesium isopropoxide), magnesium carbonate, magnesium bicarbonate, magnesium formate, magnesium acetate, magnesium trisilicates, magnesium salts of organic acids, such as fumaric acid, maleic acid, acrylic acid, methacrylic acid, itaconic acid and styrenesulfonic acid, and a combination thereof.

Other phosphate sequestrants suitable for co-administration include various examples of pharmaceutically acceptable zinc compounds are described in PCT Application No. PCT/US2005/047582, filed Dec. 29, 2005, the entire teachings of which are incorporated herein by reference. Specific suitable examples of pharmaceutically acceptable zinc compounds include zinc acetate, zinc bromide, zinc caprylate, zinc carbonate, zinc chloride, zinc citrate, zinc formate, zinc hexafluorosilicate, zinc iodate, zinc iodide, zinc iodide-starch, zinc lactate, zinc nitrate, zinc oleate, zinc oxalate, zinc oxide, calamine (zinc oxide with a small proportion of ferric oxide), zinc p-phenolsulfonate, zinc propionate, zinc salicylate, zinc silicate, zinc stearate, zinc sulfate, zinc sulfide, zinc tannate, zinc tartrate, zinc valerate and zinc ethylenebis(dithiocarbamate). Another example includes poly(zinc acrylate).

When referring to any of the above-mentioned phosphate sequestrants, it is to be understood that mixtures, polymorphs and solvates thereof are encompassed.

In some embodiments, a mixture of the phosphate sequestrants described above can be used in the invention in combination with pharmaceutically acceptable ferric or ferrous iron salts.

In other embodiments, the phosphate sequestrant used in combination crosslinked polyamine particles of the present invention is not a pharmaceutically acceptable magnesium compound. In yet other embodiments, the phosphate sequestrant used in combination with the pharmaceutically acceptable crosslinked polyamine particles is not a pharmaceutically acceptable zinc compound.

The invention also includes methods and pharmaceutical compositions directed to a combination therapy of the crosslinked polyamine particles in combination with a phosphate transport inhibitor or an alkaline phosphatase inhibitor. Alternatively, a mixture of the crosslinked polyamine particles is employed together with a phosphate transport inhibitor or an alkaline phosphatase inhibitor.

Suitable examples of phosphate transport inhibitors can be found in co-pending U.S. Publication Nos. 2004/0019113 and 2004/0019020 as well as WO 2004/085448, the entire teachings of each of which are incorporated herein by reference.

Examples of alkaline phosphatase (ALP) inhibitors may be found in, for example, U.S. Pat. No. 5,948,630, the entire teachings of which are incorporated herein by reference. Examples of alkaline phosphatase inhibitors include orthophosphate, arsenate, L-phenylalanine, L-homoarginine, tetramisole, levamisole, L-p-Bromotetramisole, 5,6-Dihydro-6-(2-naphthyl) imidazo-[2,1-b]thiazole (napthyl) and derivatives thereof. The preferred inhibitors include, but are not limited to, levamisole, bromotetramisole, and 5,6-Dihydro-6-(2-naphthyl)imidazo-[2,1-b]thiazole and derivatives thereof.

This co-administration can include simultaneous administration of the two agents in the same dosage form, simultaneous administration in separate dosage forms, and separate administration. For example, for the treatment of hyperphosphatemia, the crosslinked polyamine particles may be co-administered with calcium salts which are used to treat hypocalcemia resulting from hyperphosphatemia.

The pharmaceutical compositions of the invention can be formulated as tablets, chewable tablets, sachets, slurries, food formulations, troches, capsules, elixirs, suspensions, syrups, wafers, chewing gums or lozenges.

Preferably, the crosslinked polyamine particles or the pharmaceutical compositions comprising the crosslinked polyamine particles are administered orally. Illustrative of suitable methods, vehicles, excipients and carriers are those described, for example, in Remington's Pharmaceutical Sciences, 19^{th} ed., the contents of which is incorporated herein by reference.

Pharmaceutical compositions for use in accordance with the present invention may be formulated in conventional manner using one or more physiologically acceptable carriers comprising excipients and auxiliaries which facilitate processing of the active crosslinked polyamine particles into preparations which can be used pharmaceutically. Proper formulation is dependent upon the selected route of administration. Suitable techniques for preparing pharmaceutical compositions are well known in the art.

In some aspects of the invention, the crosslinked polyamine particles provide mechanical and thermal properties that are usually performed by excipients, thus decreasing the amount of such excipients required for the formulation. In some embodiments the crosslinked polyamine particles constitute over about 30 wt. %, for example over about 40 wt. %, over about 50 wt. %, preferably over about 60 wt. %, over about 70 wt. %, more preferably over about 80 wt. %, over about 85 wt. %, over about 90 wt. %, over about 95 wt. % or over about 99 wt. % of the composition, such as from about 80 wt. % to about 99 wt. % or from about 80 wt. % to about 95 wt. % of the composition, the remainder comprising suitable excipient(s).

In some embodiments, the dosage form of the composition is a tablet or tablets. In some embodiments, the compressibility of the tablets is strongly dependent upon the degree of hydration (moisture content) of the crosslin.ked polyamine particles. Preferably, the crosslinked polyamine particles have a moisture content of about 5% by weight or greater, more preferably, the moisture content is from about 5% to about 9% by weight, and most preferably about 7% by weight. It is to be understood that in embodiments in which the crosslinked polyamine particles are hydrated, the water of hydration is considered to be a component of the crosslinked polyamine particles.

The tablet can further comprise one or more excipients, such as hardeners, compression aids, glidants, lubricants and diluents, which are well known in the art. Suitable excipients include colloidal silicon dioxide, stearic acid, magnesium silicate, calcium silicate, sucrose, calcium stearate, glyceryl behenate, magnesium stearate, talc, microcrystalline cellulose (such as KG-1000), zinc stearate, sodium stearylfumarate, micro crystalline cellulose (cellulose derivative), lactose and starch.

In some embodiments, the tablets may be prepared by a method comprising the steps of: (1) hydrating or drying the crosslinked polyamine particles to the desired moisture level; (2) blending the crosslinked polyamine particles with any excipients; and (3) compressing the blend using conventional tableting technology to form a tablet or a tablet core. In some embodiments, the tablet or tablet core may then be further processed, such as coating.

In some embodiments, the invention relates to a stable, swallowable coated tablet, such as a tablet comprising the crosslinked polyamine particles as described above.

In one embodiment, the coating composition comprises a cellulose derivative and a plasticizing agent. The cellulose derivative is, preferably, hydroxypropylmethylcellulose (HPMC). The cellulose derivative can be present as an aqueous solution. Suitable hydroxypropylmethylcellulose solutions include those containing HPMC low viscosity and/or HPMC high viscosity. Additional suitable cellulose derivatives include cellulose ethers useful in film coating formulations. The plasticizing agent can be, for example, an acetylated monoglyceride such as diacetylated monoglyceride. The coating composition can further include a pigment selected to provide a tablet coating of the desired color. For example, to produce a white coating, a white pigment can be selected, such as titanium dioxide.

In one embodiment, a coated tablet of the invention can be prepared by a method comprising the step of contacting a tablet core, as described above, with a coating solution comprising a solvent, at least one coating agent dissolved or suspended in the solvent and, optionally, one or more plasticizing agents. In another embodiment, the coating may comprise 40 wt. % to 65 wt. % polyvinyl alcohol partially hydrolyzed, and/or 20wt. %-40 wt. % talc, and/or 10 wt. %-20 wt. % Macrogel and/or polyethylene glycol, and/or 1-5 wt. % polysorbate 80. Preferably, the solvent is an aqueous solvent, such as water or an aqueous buffer, or a mixed aqueous/organic solvent. Preferred coating agents include cellulose derivatives, such as hydroxypropylmethylcellulose. Typically, the tablet core is contacted with the coating solution until the weight of the tablet core has increased by an amount ranging from about 4% to about 6%, indicating the deposition of a suitable coating on the tablet core to form a coated tablet.

Other pharmaceutical excipients useful in some compositions of the invention include a binder, such as microcrystalline cellulose, carbopol, providone, water and xanthan gum; a flavoring agent, such as mannitol, xylitol, maltodextrin, fructose, or sorbitol; a lubricant, such as vegetable based fatty acids; and, optionally, a disintegrant, such as croscarmellose sodium, gellan gum, low-substituted hydroxypropyl ether of cellulose, sodium starch glycolate. Such additives and other suitable ingredients are well-known in the art; see, e.g., Gennaro A R (Ed.), Remington's Pharmaceutical Sciences, 19^{th} Edition.

In some embodiments, the crosslinked polyamine particles may be formed into tablets and/or coated tablets as discussed above having a tablet hardness of greater than 300N, such as from greater than 350N, from 375N to 600N, from 400N to 550N, or from 425N to 500N.

In one embodiment, the crosslinked polyamine particles are pre-formulated with a high Tg/high melting point low molecular weight excipient such as mannitol, sorbose, and sucrose in order to form a solid solution wherein the crosslinked polyamine particles and the excipient are intimately mixed. Methods of mixing such as extrusion, spray-drying, chill drying, lyophilization, or wet granulation are useful. Indication of the level of mixing is given by known physical methods such as differential scanning calorimetry or dynamic mechanical analysis.

In some embodiments the crosslinked polyamine particles of the invention may be provided as pharmaceutical compositions in the form of liquid formulations. In some embodiments, the pharmaceutical composition contains crosslinked polyamine particles dispersed in a suitable liquid excipient. Suitable liquid excipients are known in the art, see, e.g., Remington's Pharmaceutical Sciences.

In some embodiments, the pharmaceutical compositions may be in the form of a powder formulation packaged as a sachet that may be mixed with water or other ingestible liquid and administered orally as a drink (solution or suspension). In order to ensure that such formulations provide acceptable properties to the patient such as mouth feel and taste, a pharmaceutically acceptable anionic stabilizer may be included in the formulation.

Examples of suitable anionic stabilizers include anionic polymers such as: an anionic polypeptide, an anionic polysaccharide, or a polymer of one or more anionic monomers such as polymers of mannuronic acid, guluronic acid, acrylic acid, methacrylic acid, glucuronic acid glutamic acid or a combination thereof, and pharmaceutically acceptable salts thereof. Other examples of anionic polymers include cellulose, such as carboxyalkyl cellulose or a pharmaceutically acceptable salt thereof. The anionic polymer may be a homopoloymer or copolymer of two or more of the anionic monomers described above. Alternatively, the anionic copolymer may include one or more anionic monomers and one or more neutral comonomers such as olefinic anionic monomers such as vinyl alcohol, acrylamide, and vinyl formamide.

Examples of anionic polymers include alginates (e.g. sodium alginate, potassium alginate, calcium alginate, magnesium alginate, ammonium alginate, and esters of alginate), carboxymethyl cellulose, polylactic acid, polyglutamic acid, pectin, xanthan, carrageenan, furcellaran, gum Arabic, karaya gum, gum ghatti, gum carob, and gum tragacanth. Preferred anionic polymers are alginates and are preferably esterified alginates such as a C₂-C₅-diol ester of alginate or a C₃-C₅ triol ester of alginate. As used herein an “esterified alginate” means an alginic acid in which one or more of the carboxyl groups of the alginic acid are esterified. The remainder of the carboxylic acid groups in the alginate are optionally neutralized (partially or completely) as pharmaceutically acceptable salts. For example, propylene glycol alginate is an ester of alginic acid in which some of the carboxyl groups are esterified with propylene glycol, and the remainder of the carboxylic acid groups is optionally neutralized with pharmaceutically acceptable salts. More preferably, the anionic polymer is ethylene glycol alginate, propylene glycol alginate or glycerol alginate, with propylene glycol alginate even more preferred.

### EXAMPLES

### Preparation I: Crosslinked Polyallylamine Carbonate Particles Examples (1-13)

Preparation of Stock Polyallylamine Solution: 1400.00 grams of a 50% (w/w) aqueous solution of polyallylamine hydrochloride was placed in a 5 L plastic bottle. 2100 grams of deionized (DI) water was added and the resulting solution was stirred for approximately 15 minutes. While stirring, 40%-50% (w/w) NaOH solution was slowly added until a pH of approximately 10. The resulting solution was stirred until a homogenous room temperature solution was obtained.

Preparation of Crosslinked Polyallylamine: 553.1 grams of the stock polyallyamine solution, was placed in a 1 L beaker, stirred and cooled to a temperature of between 0 to 5° C. using an ice bath. 8.4 ml of epichlorohydrin was added and the solution was stirred with cooling for 1 hour. The mixture was allowed to warm to room temperature and was stirred until the formation of a gel, at which point the mixture was allowed to stand at room temperature for 17 to 18 hours.

Preparation of the Crosslinked Polyallylamine (Carbonate) Particles: At the end of the 17 to 18 hours, the gel was broken up into pieces manually, wet milled to the desired d₅₀ for constituent particle size, diluted with DI water and filtered. The gel was washed and filtered repeatedly until a conductivity of less than or equal to 1 millisiemens/cm³ (mS/cm³) was established for the suspended gel, at which point the gel was filtered. The filtered material was suspended in DI water. The pH of the suspension was adjusted to approximately 13 using from 40%-50% (w/w) aqueous NaOH solution. Again, the material was filtered, re-suspended in DI water, repeatedly, until a conductivity of less than or equal to 1 mS/cm³ was established for the suspended gel. The dry ice was placed into the suspension until a pH of between 9.0-9.9 was obtained. The gel was filtered and dried in a forced air oven at 60° C. until a constant weight was obtained (typically, between 15 and 21 hours), yielding an off-white solid gel. The off-white solid gel was removed from the forced air oven and ground using a Fritsch grinder and/or sieved to the desired size to yield crosslinked polyallylamine carbonate particles.

### Non-Competitive/Competitive Phosphate Binding Capacity

The phosphate binding of various size particles of the polymer prepared in Preparation I was tested in the absence of competing ions and in the presence of competing ions according to the procedures detailed in the Test Procedures. The particle sizes tested were determined based on particles that passed through (“−”) or were held back (“+”) by individual sieves having standard mesh sizes. For example, a particle having a size designated as −50/+80 passed through a 50 (<297 μm) mesh sieve but did not pass through an 80 (>177 μm) mesh sieve. The results of the phosphate binding test are presented in Tables 1 and 2 below.

TABLE 1
Non-Competitive Phosphate Binding Capacity
of Particles from Preparation I

| | | Bound Phosphate (mmol/g) | |
|---|---|---|---|
| Exam- | | 60 | 300 |
| ple | Particle Size Range (μm) | Minutes | Minutes |
| 1 | +50 (>297) | 4.61 | 4.66 |
| 2 | −50/+80 (<297−>177) | 4.54 | 4.53 |
| 3 | −80/+140 (<177−>105) | 4.83 | 4.92 |
| 4 | −140 | 4.88 | 4.81 |

TABLE 2
Competitive Phosphate Binding Capacity
of Particles from Preparation I

| | | Bound Phosphate (mmol/g) | |
|---|---|---|---|
| Exam- | | 60 | 300 |
| ple | Particle Size Range (μm) | Minutes | Minutes |
| 5 | +50 (>297) | 2.07 | 0.87 |
| 6 | −50/+80 (<297−>177) | 1.56 | 0.67 |
| 7 | −80/+140 (<177−>105) | 1.22 | 0.46 |
| 8 | −140 | 0.94 | 0.18 |

### Time & Particle Size Dependence of Competitive Phosphate Binding Capacity

The time dependence of bound phosphate for various size particles of crosslinked polyallylamine from Preparation I were tested in a Competitive Phosphate Binding Test as detailed in the test methods section below and compared to RENAGEL®. The results are presented in Table 3 below:

TABLE 3
Time & Particle Size Dependence of Competitive
Phosphate Binding Capacity

| | | Phosphate Bound (mmol/g) | | | |
|---|---|---|---|---|---|
| | | 20 | 40 | 60 | 300 |
| Exam- | | min- | min- | min- | min- |
| ple | Particle Size Range (μm) | utes | utes | utes | utes |
| A | RENAGEL ® | 1.47 | 0.88 | 0.32 | 0.00 |
| 9 | −230/+325 (<63−>44) | 1.08 | 0.54 | 0.40 | 0.27 |
| 10 | −140/+170 (<105−>88) | 1.03 | 0.89 | 0.47 | 0.20 |
| 11 | −20/+25 (<841−>707) | 0.98 | 1.37 | 1.26 | 0.42 |
| 12 | −10/+12 (<2000−>1680) | 0.63 | 1.04 | 1.09 | 0.57 |
| 13 | +4 (>4760) | 0.72 | 1.20 | 1.33 | 0.75 |

### Preparation II: Crosslinked Polyallylamine Carbonate Particles (Examples 14-19)

Polyallylamine carbonate was prepared as in Preparation I with the following procedural differences: 1) at the end of the 17 to 18 hours, the room temperature crosslinked polyallylamine gel was not wet milled to a desired constituent particle size and was instead broken into pieces manually, diluted with DI water and filtered; and 2) the off-white solid gel was removed from the forced air oven and ground using a potato masher against a hard surface to yield crosslinked polyallylamine carbonate particles. These particles were fractionated into aggregate particles having the sizes noted in Table 4A using 20 and 50 mesh sieves.

### Preparation III. Crosslinked Polyallylamine Carbonate Particles (Examples 20-21)

Polyallylamine carbonate was prepared as in Preparation II with the following procedural difference: the polyallylamine stock solution was at room temperature when the epichlorohydrin was added, instead of at 0 to 5° C.

### Preparation IV: Crosslinked Polyallylamine Carbonate Particles (Examples 22-27)

660 g of sevelamer carbonate having an average particles size of 90 μm, noted as the Constituent Particles Size in Tables 4A and 4B, was suspended in 12 L of DI water and stirred with an overhead stirrer for 18 hours. The resulting gel was collected on a filter under vacuum, was placed in a tray and dried at 65° C. for 17 hours in a forced air oven. The dried solid gel was removed from the forced air oven and milled using an electric grinder to yield crosslinked polyallylamine carbonate particles. These particles were fractionated into particles having the sizes noted in Table 4A using 20 and 50 mesh sieves.

### Preparation V: Crosslinked Polyallylamine Carbonate Particles (Examples 28-33 and 50-54)

Polyallylamine carbonate was prepared as in Preparation I with the following procedural differences: 1) At the end of the 17 to 18 hours, the gel was manually broken up and wet milled using a Waring® HGBSS blender (Model No. HGBSSSS6) for 14 seconds. The size of the predominant particles resulting from the wet mill blend is noted as the Constituent Particles Size in Tables 4A and 4B. These particles were diluted with DI water and filtered. 2) The dried solid off-white gel was removed from the forced-air oven and milled using (A) a potato masher, (B) a mortar and pestle or (C) a pulsed electric coffee grinder as indicated in the table to yield crosslinked polyallylamine carbonate particles. These particles were fractionated into particles having the sizes noted in Tables 4A and 4B using 20 and 50 mesh sieves.

Preparation VI: Crosslinked Polyallylamine Carbonate Particles formed from Fine Particles (Example 34)

Polyallylamine carbonate was prepared as in Preparation V with the following procedural differences: the fine particles that passed through the sieves from Preparation V were suspended in DI water and stirred. The resulting gel was filtered, placed in a tray and dried at 65° C. for 17 hours in a forced air oven. The dried solid gel was removed from the forced air oven and milled using an electric coffee grinder to yield crosslinked polyallylamine carbonate particles. These particles were fractionated into particles having the sizes noted in Table 4A using 20 and 50 mesh sieves.

Preparation VII: Crosslinked Polyallylamine Carbonate Particles formed in the Presence of Heptanes (Examples 35-36)

A 100 ml jacketed reactor was charged with 40 g of 50% (w/w) aqueous polyallylamine hydrochloride and 60 g of water. A homogenizer was inserted into the mixture and used to mix the two layers at 5,000 rpm. During homogenization, 10.6 g of 50% (w/w) aqueous sodium hydroxide was added to adjust the pH to approximately 10. After adjusting the pH, the reaction mixture was cooled to approximately 3° C., stirring occasionally using the homogenizer. With the homogenizer on, 1.7 ml of epichlorohydrin followed by 10 ml of heptanes were injected into the solution below the surface adjacent to the homogenizer head. After 5 minutes, homogenization was discontinued and magnetic stirring was initiated. The reaction mixture became translucent. One hour after the epichlorohydrin addition, the reaction mixture was allowed to warm slowly to room temperature. After curing overnight, 7 ml of heptanes had separated from the resulting crosslinked polyallylamine gel and was poured off. The gel was cut into 1 cm³ pieces which were suspended in 800 ml of water, stirred for 1 hour, and then filtered. The gel was washed and filtered repeatedly until the conductivity had dropped to below 1.0 mS/cm³. The gel was re-suspended and 50% (w/w) aqueous NaOH added until the pH was approximately 13. The mixture was filtered and washed repeatedly until the conductivity was below 1.0 mS/cm3. The gel was re-suspended again and dry ice was added slowly to the mixture until the pH was approximately 9.0 to 9.9. The mixture was filtered and dried in a 60° C. forced-air drying oven for 16 hours. The dried crosslinked polyallylamine carbonate gel was ground using a mortar and pestle to yield crosslinked polyallylamine carbonate particles. These particles were fractionated into particles having the sizes noted in Table 4A using 20 and 50 mesh sieves.

Preparation VIII: Crosslinked Polyallylamine Carbonate Particles formed in the Presence of Nitrogen (Example 37)

Crosslinked polyallylamine carbonate particles were prepared as in Preparation VII with the following procedural differences: 1) instead of adding heptanes, a glass pipette with a 3 mm opening was inserted into the reaction mixture, at the time the epichlorohydrin was added, at a point near the bottom of the vessel and N₂ was passed through the mixture at a high flow rate, such that the surface of the reaction mixture seethed and the solution quickly became opaque with numerous fine bubbles; 2) after curing overnight, the nitrogen was turned off, the pipette was removed and the resulting crosslinked polyallylamine gel was suspended in 800 ml of water (with no breaking beyond incidental breaking as a result of removing the gel from the reactor) stirred for 30 minutes, and then filtered.

### Preparation IX. Crosslinked Polyallylamine Carbonate Particles (Examples 38-44)

1400.00 grams of 50% (w/w) aqueous solution of polyallylamine hydrochloride was placed in a 5 L plastic bottle. 2100 grams of DI water was added and the resulting solution was stirred for 15 minutes. While stirring, 370.76 grams of 50% (w/) aqueous NaOH solution was slowly added. The resulting solution was stirred and cooled to 0° C. 8.4 ml of epichlorohydrin was added to the cooled solution and stirred at 0° C. for one hour. The mixture was allowed to warm to room temperature and was stirred until the formation of a gel, at which point the mixture was allowed to stand at room temperature for 17 to 18 hours.

At the end of the 17 to 18 hours, the gel was broken up into pieces manually, diluted with DI water, the resulting slurry was poured into a 5L container and DI water was used to fill the remainder of the container. The material was filtered, re-suspended in DI water, and filtered, repeatedly, until a conductivity of less than or equal to 1 mS/cm³ was established. The filtered material was suspended in DI water. The pH of the suspension was adjusted to 13.00 using 40% (w/w) aqueous NaOH solution. Again, the material was filtered, re-suspended in DI water, and filtered, repeatedly, until a conductivity of less than or equal to 1 mS/cm³ was established. Subsequently, dry ice was placed into the suspension until a pH of approximately 9.0 to 9.9 was obtained. The mixture was filtered and the resultant gel was placed in a tray and dried at 65° C. for 17 hours in a forced air drying oven. The dried gel was removed from the oven and ground and sieved to the desired Constituent Particles d₅₀ noted in Table 4B. The ground gel was re-suspended in DI water, was filtered and placed in a tray and dried at 65° C. for 17 hours in a forced air drying oven. The dried gel was removed from the oven and milled using a pulsed electric coffee grinder to yield crosslinked polyallylamine carbonate particles. These particles were fractionated into particles having the sizes noted in Table 4B using 20 and 50 mesh sieves.

### Preparation X: Crosslinked Polyallylamine Carbonate Particles (Examples 45-49)

Polyallylamine carbonate was prepared as in Preparation V up through the formation of the dried crosslinked polyallylamine carbonate gel, which in this preparation was milled using a pulsed electric coffee grinder and sieved to the Constituent Particles d₅₀ noted in Table 4B (as opposed to the particles resulting from the wet milling prior to carbonating the crosslinked polyallylamine gel, as in Preparation V); 2) after the milling and sieving to the various distributions, the sieved crosslinked polyallylamine carbonate constituent particles were re-suspended, dried for 15 hours, and milled using a pulsed electric coffee grinder to yield crosslinked polyallylamine carbonate particles. These particles were fractionated into particles having the sizes noted in Table 4B using 20 and 50 mesh sieves.

### Phosphate Binding and Mean Gray Value of Particles

The Constituent Particles d₅₀, Competitive Phosphate Binding and Mean Gray Value, measured in accordance with the Test Methods, of the crosslinked polyallylamine particles prepared according to Preparations II-X (Examples 14-50) are presented in Tables 4A & 4B.

TABLE 4A
Properties of Particles - Examples 14-37

| | | | Competitive | | |
|---|---|---|---|---|---|
| | Constituent | | Phosphate | Mean Grey | |
| Exam- | Particles | Particle Size d₁₀, | Binding | Value | |
| ple | d₅₀ (μm) | d₅₀, d₉₀ (μm)^{a} | (mmol/g)^{d} | (Std. Dev.) | Preparation |
| 14 | >841^{c} | d₁₀ = 527.24 | 0.75 | 172.38 | II |
| | | d₅₀ = 792.341 | | (9.09) | |
| | | d₉₀ = 1225.14 | | | |
| 15 | >841^{c} | d₁₀ = 664.602 | 1.34 | 178.02 | II |
| | | d₅₀ = 954.946 | | (8.00) | |
| | | d₉₀ = 1374.233 | | | |
| 16 | >841^{c} | d₁₀ = 627.836 | 0.27 | 175.68 | II |
| | | d₅₀ = 940.79 | | (4.94) | |
| | | d₉₀ = 1406.73 | | | |
| 17 | >841^{c} | ND | 1.06 | ND | II |
| 18 | >841^{c} | d₁₀ = 601.559 | 0.27 | 174.29 | II |
| | | d₅₀ = 865.761 | | (3.90) | |
| | | d₉₀ = 1269.392 | | | |
| 19 | >841^{c} | ND | 0.00 | 166.07 | II |
| | | | | (3.29) | |
| 20 | >841^{c} | d₁₀ = 578.831 | 0.01 | 164.57 | III |
| | | d₅₀ = 869.066 | | (8.15) | |
| | | d₉₀ = 1327.781 | | | |
| 21 | >841^{c} | d₁₀ = 592.464 | 0.13 | 157.67 | III |
| | | d₅₀ = 891.951 | | (1.69) | |
| | | d₉₀ = 1356.902 | | | |
| 22 | 90^{a} | d₁₀ = 381.320 | 1.75 | ND | IV |
| | | d₅₀ = 698.993 | | | |
| | | d₉₀ = 1085.514 | | | |
| 23 | 90^{a} | d₁₀ = 607.786 | 1.73 | 201.74 | IV |
| | | d₅₀ = 975.70 | | | |
| | | d₉₀ = 1400 | | | |
| 24 | 90^{a} | d₁₀ = 572.908 | 1.84 | 203.43 | IV |
| | | d₅₀ = 865.35 | | | |
| | | d₉₀ = 1332.776 | | | |
| 25 | 90^{a} | d₁₀ = 661.227 | 1.64 | ND | IV |
| | | d₅₀ = 989.74 | | | |
| | | d₉₀ = 1446.809 | | | |
| 26 | 90^{a} | d₁₀ = 506.55 | 1.87 | 200.43 | IV |
| | | d₅₀ = 798.75 | | | |
| | | d₉₀ = 1295.136 | | | |
| 27 | 90^{a} | d₁₀ = 478.834 | 1.21 | 194.94 | IV |
| | | d₅₀ = 798.575 | | | |
| | | d₉₀ = 1335.28 | | | |
| 28 | 120 and 400^{b} | d₁₀ = 611.209 | 1.34 | 193 | V-A |
| | | d₅₀ = 916.173 | | (1.74) | |
| | | d₉₀ = 1380.354 | | | |
| 29 | 120 and 400^{b} | ND | 1.55 | 195.54 | V-A |
| | | | | (3.59) | |
| 30 | 120 and 400^{b} | d₁₀ = 635.185 | 1.62 | 194.84 | V-B |
| | | d₅₀ = 949.015 | | (3.88) | |
| | | d₉₀ = 1413.625 | | | |
| 31 | 120 and 400^{b} | d₁₀ = 634.264 | 1.63 | 198.47 | V-B |
| | | d₅₀ = 949.535 | | (2.75) | |
| | | d₉₀ = 1414.496 | | | |
| 32 | 120 and 400^{b} | ND | 1.72 | 199.5 | V-B |
| | | | | (0.34) | |
| 33 | 120 and 400^{b} | d₁₀ = 375.365 | 1.91 | ND | V-B |
| | | d₅₀ = 720.936 | | | |
| | | d₉₀ = 1108.081 | | | |
| 34 | 105-297^{c} | ND | 1.79 | ND | VI |
| 35 | ND | d₁₀ = 635.649 | 1.5 | ND | VII |
| | | d₅₀ = 946.459 | | | |
| | | d₉₀ = 1409.24 | | | |
| 36 | ND | d₁₀ = 637.895 | 1.71 | ND | VII |
| | | d₅₀ = 954.202 | | | |
| | | d₉₀ = 1418.952 | | | |
| 37 | ND | ND | 2.21 | ND | VIII |

Table Notes:
^{a}Particle size was measured by Malvern Mastersizer and is reported as a mean size based on volume percent.
^{b}Particle size was measured by microscopy and was bimodal, with the means for the two modes reported.
^{c}Particle size was measured by sieve and is reported according to the size in microns corresponding to the mesh or meshes used.
^{d}The reported Competitive Phosphate Binding is measured at 60 minutes in accordance with the test procedures.
ND means Not Determined.

TABLE 4B
Properties of Particles - Examples 38-50

| | | | Competitive | |
|---|---|---|---|---|
| | Constituent | | Phosphate | |
| | Particles | Particle Size d₁₀, | Binding | |
| Sample | d₅₀ (μm) | d₅₀, d₉₀ (μm) ^{a} | (mmol/g)^{d} | Preparation |
| 38 | 500-420^{c} | d₁₀ = 402.767 | 0.53 | IX |
| | | d₅₀ = 639.033 | | |
| | | d₉₀ = 1056.395 | | |
| 39 | 354-297^{c} | d₁₀ = 409.724 | 0.6 | IX |
| | | d₅₀ = 751.769 | | |
| | | d₉₀ = 1371.072 | | |
| 40 | 297-210^{c} | d₁₀ = 467.972 | 0.5 | IX |
| | | d₅₀ = 822.724 | | |
| | | d₉₀ = 1432.177 | | |
| 41 | 210-105^{c} | d₁₀ = 602.852 | 1.06 | IX |
| | | d₅₀ = 902.221 | | |
| | | d₉₀ = 1360.314 | | |
| 42 | 105-74^{c} | d₁₀ = 638.412 | 0.87 | IX |
| | | d₅₀ = 1002.203 | | |
| | | d₉₀ = 1511.621 | | |
| 43 | 210-74^{c} | d₁₀ = 591.134 | 0.63 | IX |
| | | d₅₀ = 969.86 | | |
| | | d₉₀ = 1520.841 | | |
| 44 | <53^{c} | d₁₀ = 591.134 | 0.34 | IX |
| | | d₅₀ = 967.136 | | |
| | | d₉₀ = 1516.632 | | |
| 45 | 297-210^{c} | d₁₀ = 471.336 | 1.19 | X |
| | | d₅₀ = 689.076 | | |
| | | d₉₀ = 1030.711 | | |
| 46 | 210-105^{c} | d₁₀ = 597.004 | 1.26 | X |
| | | d₅₀ = 862.014 | | |
| | | d₉₀ = 1273.268 | | |
| 47 | 105-74^{c} | d₁₀ = 631.069 | 1.61 | X |
| | | d₅₀ = 913.429 | | |
| | | d₉₀ = 1334.224 | | |
| 48 | 74-53^{c} | d₁₀ = 616.825 | 1.3 | X |
| | | d₅₀ = 889.167 | | |
| | | d₉₀ = 1300.21 | | |
| 49 | <53^{c} | d₁₀ = 602.852 | 0.76 | X |
| | | d₅₀ = 902.221 | | |
| | | d₉₀ = 1360.314 | | |
| 50 | 120 and 400^{b} | d₁₀ = 589.549 | 1.36 | V-C |
| | | d₅₀ = 881.346 | | |
| | | d₉₀ = 1335.277 | | |
| 51 | 120 and 400^{b} | ND^{e} | 1.68 | V-B |
| 52 | 120 and 400^{b} | ND^{e} | ND | V-B |

Table Notes:
^{a} Particle size was measured by Malvern Mastersizer and is reported as a mean size based on volume percent.
^{b}Particle size was measured by microscopy and was bimodal with the means for the two modes reported.
^{c}Particle size was measured by sieve and is reported according to the size in microns corresponding to the mesh or meshes used.
^{d}The reported Competitive Phosphate Binding is measured at 60 minutes in accordance with the test procedures.
^{e}Though these particles were not sized using any of the above procedures, they were fractionated to a mesh size of −20/+50.

### Time & Particle Size Dependence of Competitive Phosphate Binding Capacity and Bile Acid Binding Capacity

The time & particle size dependence of the Competitive Phosphate Binding Capacity and the Bile Acid Binding Capacity determined according to the procedures detailed in the Test Procedures of crosslinked polyallylamine from Example 51 were compared to the Competitive Phosphate Binding Capacity and the Bile Acid Binding Capacity of Example B, particles of sevelamer carbonate having a d₅₀ of 90 μm. The results are presented in Tables 5 & 6 below:

TABLE 5
Time & Particle Size Dependence of Competitive
Phosphate Binding Capacity

| | | Phosphate Bound (mmol/g) | | | | | |
|---|---|---|---|---|---|---|---|
| | | 20 | 40 | 60 | 120 | 200 | 300 |
| Exam- | Particles | min- | min- | min- | min- | min- | min- |
| ple | d₅₀ | utes | utes | utes | utes | utes | utes |
| B | 90 μm | 1.22 | 0.92 | 0.62 | 0.3 | 0.24 | 0.12 |
| 51 | ND | 0.87 | 1.50 | 1.68 | 1.54 | 1.21 | 0.82 |

TABLE 6
Time & Particle Size Dependence of Bile Acid Binding Capacity

| | | Bile Acid Bound (mmol/g) | | | | | |
|---|---|---|---|---|---|---|---|
| | | 20 | 40 | 60 | 120 | 200 | 300 |
| Exam- | Particles | min- | min- | min- | min- | min- | min- |
| ple | d₅₀ | utes | utes | utes | utes | utes | utes |
| B | 90 μm | 5.98 | 7.25 | 7.77 | 8.36 | 8.53 | 8.54 |
| 51 | ND | 1.93 | 3.23 | 3.77 | 5.26 | 6.32 | 7.06 |

### In Vivo Urinary Phosphorous Reduction and In Vivo Fecal Bile Acid Increase

The in vivo Urinary Phosphorous Reduction determined according to the procedure detailed in the Test Procedures of 0.5 wt. % crosslinked polyallylamine from Example 51 was compared to the in vivo Urinary Phosphorous Reduction of 0.5 wt. % of Example B, to a 0.5 wt% Cellulose negative control (“Example C”) and to 0.25 wt. % (“Example D”) and 0.5 wt. % (‘Example “E”) of sevelamer hydrochloride having a d₅₀ of between 20 μm to 100 μm. The results are presented in Table 7 below.

The in vivo Fecal Bile Acid Increase determined according to the procedure detailed in the Test Procedures of 1 wt. %, 2 wt. % and 4 wt. % crosslinked polyallylamine from Example 52 was compared to a 4 wt. % Cellulose negative control (“Example F”), to 1 wt. % (“Example G”), 2 wt. % (Example “H”) and 4 wt. % ('Example “I”) of sevelamer hydrochloride having a d₅₀ of between 20 μm to 100 μm. and to 1 wt. % (“Example J”), 2 wt. % (Example “K”) and 4 wt. % ('Example “L”) of sevelamer carbonate having an d₅₀ of 90 μm. The results are presented in Table 8.

TABLE 7
In vivo Urinary phosphorous reduction

| | | |
|---|---|---|
| | | % Reduction |
| | Mean Urinary Phosphorous in | Relative to |
| Test Article | mg/day (Standard Deviation) | Example D |

| | | | |
|---|---|---|---|
| Example C | 14.1 | (4.4) | — |
| Example D | 11.6 | (3.5) | 17.7% |
| Example E | 7.2 | (3.4) | 48.9% |
| Example B | 6.5 | (3.3) | 53.9% |
| Example 51 | 4.9 | (2.8) | 65.2% |

TABLE 8
In vivo Fecal Bile Acid Increase

| | | | % Increase |
|---|---|---|---|
| | In vivo Fecal Bile Acid in | | Relative to |
| Test Article | mg/day (Standard Deviation) | | Example F |
| Example F | 25.89 | (3.46) | — |
| Example G | 96.13 | (13.48) | 271% |
| Example H | 106.63 | (35.67) | 312% |
| Example I | 99.83 | (20.95) | 286% |
| Example J | 84.91 | (19.17) | 228% |
| Example K | 100.28 | (17.55) | 287% |
| Example L | 144.29 | (16.14) | 457% |
| Example 52 - 1 wt. % | 103.38 | (31.36) | 299% |
| Example 52 - 2 wt. % | 114.01 | (21.29) | 340% |
| Example 52 - 4 wt. % | 123.75 | (14.64) | 378% |

### Particle Acid Stability (Examples 53 and 54)

Preparation of the Crosslinked Polyallylamine (Carbonate) Particles: Two lots of crosslinked polyallylamine were prepared according to Preparation I (i.e., Preparation of Stock Polyallyamine Solution and Preparation of Crosslimked Polyallylamine). At the end of the 17 to 18 hours, each lot of gel was manually broken up and wet milled using a Waring® HGBSS blender (Model No. HGBSSSS6) for 14 seconds diluted with DI water and filtered. The particles of gel, for each lot, were washed and filtered repeatedly until a conductivity of less than or equal to 1 millisiemens/cm³ (mS/cm³) was established for the suspended gel, at which point the gel was filtered. The filtered material was suspended in DI water. The pH of the suspension was adjusted to approximately 13 using from 40%-50% (w/w) aqueous NaOH solution. Again, the material was filtered, re-suspended in DI water, repeatedly, until a conductivity of less than or equal to 1 mS/cm³ was established for the suspended gel. The dry ice was placed into the suspension until a pH of between 9.0-9.9 was obtained. The gel was filtered and dried in a forced air oven at 60° C. until a constant weight was obtained (typically, between 15 and 21 hours), yielding an off-white solid gel. The off-white solid gel was removed from the forced air oven and ground using a mortar and pestle to yield crosslinked polyallylamine carbonate particles having the properties set forth in Tables 9 & 10, below.

### Particle Size Stability

Samples of the crosslinked polyamine from Examples 53 and 54 were taken after the final step of the preparation process, i.e. at t=0 and after acid treatment, in accordance with the Wet Particle Size & Distribution (in Acid) test method in the Test Procedures, of both the non-cured particles and the cured partices (i.e., particles kept for 3 weeks in a 60° C. oven) and are presented Table 9 in below.

TABLE 9
Particle Size Acid Stability

| | Volume | Wet Particle | Wet Particle | Increase in Wet Particle |
|---|---|---|---|---|
| | Weighted Mean | Size (non-cured) | Size (cured) | Size After Acid Treatment |
| Exam- | Particle Size | After Acid | After Acid | (cured particle to |
| ple | at t = 0 (μm) | Treatment (μm) | Treatment (μm) | non-cured particle) |
| 53 | 838 | 191 | 328 | 1.7 fold |
| 54 | 800 | 196 | 426 | 2.2 fold |

### Competitive Phosphate Binding Stability

Samples of the crosslinked polyamine from Examples 53 and 54 non-heat treatment (i.e., at time t=0) and cured (particles after 3 weeks at 60° C.) were tested for competitive phosphate binding according to the procedure detailed in the Test Procedures, before and after treatment in 0.2 N HCl for one hour. The results are provided in Table 10 below with the % competitive phosphate binding retained by the acid treated samples relative to the non-acid treated samples.

TABLE 10
Acid Stability of Competitive Phosphate Binding

| | | | Competitive | | | |
|---|---|---|---|---|---|---|
| | | | Phosphate | | % Binding | |
| | | | Binding | | Retained | |
| Exam- | | Acid | 60 | 120 | 60 | 120 |
| ple | Cured | Treatment | mins. | mins. | mins. | mins. |
| 53 | None | Before | 1.99 | 1.76 | 82 | 61 |
| | | After | 1.63 | 1.08 | | |
| | Cured | Before | 1.69 | 1.77 | 93 | 65 |
| | | After | 1.58 | 1.15 | | |
| 54 | None | Before | 1.9 | 1.54 | 78 | 55 |
| | | After | 1.49 | 0.84 | | |
| | Cured | Before | 1.61 | 1.61 | 101 | 81 |
| | | After | 1.62 | 1.31 | | |

### Low Temperature Drying

Example 55: 10 g of constituent particles of sevelamer carbonate having a mean particle size of 90 μm were hydrated in 50 ml of water and a sample was tested for % Loss on Drying (% LOD) as described in the Test Procedures, giving a % LOD of 82%. The wet sevelamer carbonate was sieved using a 1.4 mm sieve. A first portion was dried at room temperature for seven days in a desiccator using P₂O₅ as a desiccant. After drying the material was acid treated, 250 mg of the dry material was placed in 5 ml (1 N HCl) and mixed on an orbital shaker at room temperature for two hours. The resulting material fell completely apart. A second portion of the sieved wet sevelamer carbonate was (instead of being dried for seven days at room temperature) dried on a drying tray, in a forced-air hot oven, having a 1 cm bed height at 110° C. for 4 hours. This material was then acid treated, as above, and resulted in particles, as measured by a Malvern Mastersizer, with a volume weighted mean (VWM) size of 336 μm.

Examples 56 & 57: 10 g of constituent particles of sevelamer carbonate having a mean particle size of 90 μm were hydrated in 50 ml of water and a sample was tested for % Loss on Drying (% LOD) as described in the Test Procedures, giving a % LOD of 82%. The wet material was sieved using a 1.4 mm sieve. Two separate samples were taken, Example 56 and Example 57. The samples were seperately dried by placing them on a drying tray having a 1 cm bed height and was dried at 110° C., under a 400 to 500 mbar vacuum with a nitrogen purge with agitation every approximately 0.5 hour for 30 seconds at 50 rpm, for 4 hours, for Example 56, and only 1 hour, for Example 57, at which point the sample material had a % LOD of 1.2% (Example 56) and % LOD of 2.1% (Example 57). Example 57 also had a % Carbonate of 20.7. The samples were then passed through a −20/+50 mesh sieve. A 200 mg sieved portion of Example 56 and 57 were measured for Volume Weighted Mean, in accordance with the Wet Particle Size & Distribution (in Acid) test method, and recorded to be 622 μm (Example 56) and 400 μm (Example 57). A separate seived portion for each of the samples were tested for competitive phosphate binding at 60 minutes and 120 minutes. A further sieved portion for each sample of the dried material was acid treated, the particles were placed in 0.2 N HCl and mixed on an orbital shaker for 2 hours, at which point the competitive phosphate binding was measured at 60 minutes and 120 minutes. The results of the tests are presented in Table 11 along with the corresponding competitive phosphate binding measurements for a reference standard that is prepared in an identical fashion except that the material was not cured it was placed in a drying tray having a 1 cm bed height and was dried at a temperature not exceeding 60° C. for until the material had a % LOD of less than 3 (approximately 16-17 hours), under a 400 to 500 mbar vacuum with a nitrogen purge, with agitation every approximately 0.5 hour for 30 seconds at 50 rpm.

TABLE 11
Acid Stability of Competitive Phosphate Binding

| | | Competitive Phosphate Binding | | | | | |
|---|---|---|---|---|---|---|---|
| Exam- | | 60 | 120 | Ref. std @ | Ref. std @ | Normalized* @ | Normalized* @ |
| ple | Treatment | mins. | mins. | 60 mins. | 120 mins. | 60 mins. | 120 mins. |
| 56 | No acid | 1.85 | 1.78 | 1.57 | 1.14 | 1.18 | 1.56 |
| | Acid treated | 1.97 | 1.64 | 0.99 | 0.86 | 1.98 | 1.91 |
| 57 | No acid | 1.98 | 1.99 | 1.73 | 1.55 | 1.14 | 1.28 |
| | Acid treated | 1.55 | 1.31 | 0.99 | 0.86 | 1.56 | 1.52 |

Table Notes:
*The normalized values are calculated by dividing the actual value by the reference value (i.e., actual value/reference value).

### Effect of Hydration on Yield and Acid Stability

Examples 58-61: 10 g of constituent particles of sevelamer carbonate having a mean particle size of 90 μm were hydrated with various amounts of water to yield samples having the % LOD shown in Table 12. The wet material was sieved using a 1.4 mm sieve. The sieved material was placed in a drying tray having a 1 cm bed height and was dried at 110° C. for 4 hours, under a 400 to 500 mbar vacuum with a nitrogen purge, with agitation every approximately 0.5 hour for 30 seconds at 50 rpm. The dried material was sieved and the yield of particles having a particle size that was −20/+50 mesh by weight was determined for each Example. A portion of the sieved material for each Example was tested for competitive phosphate binding at 60 minutes and 120 minutes. A further sample from each Example was Acid Treated, the particles were placed in 0.2 N HCl and mixed on an orbital shaker for 2 hours, at which point the competitive phosphate binding was measured at 60 minutes and 120 minutes. The results of the tests are presented in Table 12.

TABLE 12
Effect of Varying Hydration on Acid Stability of Crosslinked Polyamine Particles

| | | | | | | % Retention of | |
|---|---|---|---|---|---|---|---|
| | | | | Competitive | | Activity after Acid | |
| Exam- | | | | Phosphate Binding | | Treatment | |
| ple | % LOD | Yield | Conditions | 60 min | 120 min | 60 min | 120 min |
| 58 | 25% | 19% | No acid | 1.54 | 0.79 | 89.6% | 98.7% |
| | | | Acid treated | 1.38 | 0.78 | | |
| 59 | 35% | 58% | No acid | 1.88 | 1.3 | 88.8% | 60.8% |
| | | | Acid treated | 1.67 | 0.79 | | |
| 60 | 45% | 67% | No acid | 2.15 | 1.64 | 90.7% | 65.8% |
| | | | Acid treated | 1.95 | 1.08 | | |
| 61 | 80% | 83% | No acid | 2.02 | 2.06 | 95% | 79.6% |
| | | | Acid treated | 1.92 | 1.64 | | |

Example 62: A sevelamer carbonate gel, that had never been dried, having a 80% LOD was dried in accordance with the following procedure. The wet material was wet co-milled with a 813 μm screen at 1300 rpm. After it was co-milled, the sample material was dried, in a fluid bed dryer having an inlet air temperature of 65° C. and a 40 cubic foot per minute (CFM), to about 30% LOD (approximately 70 minutes). It was then co-milled again with a 1575 μm screen at 1300 rpm. The material was then placed in a dryer manufactered by Littleford Day (model M5) and was dried at 110° C. for 4 hours to approximately 2-3% LOD, under a 400 to 500 mbar vacuum with a nitrogen purge, with agitation approximately every 0.5 hour for 30 seconds at 50 rpm. The dried material was sieved through a −20/+50 mesh screen. A portion of the sieved particles were then cured in a dryer manufactered by Littleford Day (model M5) at 110° C. for 4 hours, under a 400 to 500 mbar vacuum with a nitrogen purge, with agitation approximately every 0.5 hour for 30 seconds at 50 rpm. The measured properties of the sample in both the uncured and cured state are presented in Table 13A.

Additional samples were prepared in accordance with the method set forth in Example 62. The properties of these samples are provided in Table 13B.

TABLE 13A
Results for Example 62

| Yield | | | 75% |
|---|---|---|---|
| Soluble Oligomers of Cured (%) | | | 0.08 |
| Granule Appearance | | | Off White |
| Total Drying Time (hr.) | | | ~9.5 |
| Un-cured Volume Weighted Mean* (μm) | | | 360 |
| Cured - Volume Weighted Mean* (μm) (4-hrs.) | | | 454 |
| Competitive Phosphate | Uncured | 60 min. | 1.78 |
| Binding | | 120 min. | 1.59 |
| | Cured for | 60 min. | 2.04 |
| | 4 hrs. | 120 min. | 1.81 |

*Table Note:
Volume Weighted Mean measured in accordance with the Wet Particle Size & Distribution (in Acid) test method.

TABLE 13B
Samples in Accordance with the Process of Example 62

| Un-cured Volume Weighted Mean* (μm) | | | 501 |
|---|---|---|---|
| Cured - Volume Weighted Mean* (μm) (4-hrs.) | | | 598 |
| Competitive Phosphate | Uncured | 60 min. | 2.13 |
| Binding | | 120 min. | 1.74 |
| | Cured for | 60 min. | 2.21 |
| | 4 hrs. | 120 min. | 1.75 |

*Table Note:
Volume Weighted Mean measured in accordance with the Wet Particle Size & Distribution (in Acid) test method.

Example 63: A sevelamer carbonate gel, that had never been dried, having a 80% LOD was dried in accordance with the following procedure. The material was placed in a dryer manufactered by Littleford Day (model M5) and was dried at 110° C. for approximately 6.5 hours (approximately 30% LOD), under a 400 to 500 mbar vacuum with a nitrogen purge, with continuous agitation 30 rμm. It was then co-milled with a 1575 μm screen at 1300 rpm. The milled material was placed in a dryer manufactered by Littleford Day (model M5) and dried at 110° C. for 4 hours to approximately 2-3% LOD, under a 400 to 500 mbar vacuum with a nitrogen purge, with agitation approximately every 0.5 hour for 30 seconds at 50 rpm The dried material was sieved through a −20/+50 mesh screen. A portion of the sieved particles were then cured in a dryer manufactered by Littleford Day (model M5) at 110° C. for 4 hours, under a 400 to 500 mbar vacuum with a nitrogen purge, with agitation approximately every 0.5 hour for 30 seconds at 50 rpm. A further portion was maintained in the dryer to be cured at 110° C. for 2 additional hours (for a total of 6 hours). The measured properties of the sample in both the uncured and cured state are presented in Table 14A.

Additional samples were prepared in accordance with the method set forth in Example 63. The properties of these samples are provided in Table 14B.

TABLE 14A
Results for Example 63

| Yield | | | 50% |
|---|---|---|---|
| Granule Appearance | | | Off White |
| Total Drying Time (hr.) | | | ~14.5 |
| Un-cured Volume Weighted Mean* (μm) | | | 235 |
| Cured - Volume Weighted Mean* (μm) (4-hrs.) | | | 475 |
| Cured - Volume Weighted Mean* (μm) (6-hrs.) | | | 589 |
| Competitive Phosphate | Uncured | 60 min. | 1.50 |
| Binding | | 120 min. | 1.07 |
| | Cured for | 60 min. | 1.94 |
| | 4 hrs. | 120 min. | 1.40 |

*Table Note:
Volume Weighted Mean measured in accordance with the Wet Particle Size & Distribution (in Acid) test method.

TABLE 14B
Samples in Accordance with the Process of Example 63

| Un-cured Volume Weighted Mean* (μm) | | | 405 |
|---|---|---|---|
| Cured - Volume Weighted Mean* (μm) (4-hrs.) | | | 462 |
| Competitive Phosphate | Uncured | 60 min. | 1.71 |
| Binding | | 120 min. | 1.42 |
| | Cured for | 60 min. | 1.87 |
| | 4 hrs. | 120 min. | 1.34 |

*Table Note:
Volume Weighted Mean measured in accordance with the Wet Particle Size & Distribution (in Acid) test method.

Example 64: A sevelamer carbonate gel, that had never been dried, having a 80% LOD was dried in accordance with the following procedure. The material was placed in a dryer manufactered by Littleford Day (model M5) and was dried at 110° C. for 4.5 hours (approximately 30% LOD), under a 400 to 500 mbar vacuum with a nitrogen purge, with continuous agitation 100 rpm. It was then co-milled with a 1575 μm screen at 1300 rpm. The milled material was placed in a dryer manufactered by Littleford Day (model M5) and dried at 110° C. for 4 hours (approximately 2-3% LOD), under a 400 to 500 mbar vacuum with a nitrogen purge, with agitation approximately every 0.5 hour for 30 seconds at 50 rpm. The dried material was sieved through a −20/+50 mesh screen. A portion of the sieved particles was then cured in a dryer manufactered by Littleford Day (model M5) at 110° C. for 4 hours, under a 400 to 500 mbar vacuum with a nitrogen purge, with agitation approximately every 0.5 hour for 30 seconds at 50 rpm. The measured properties of the sample in both the uncured and cured state are presented in Table 15.

TABLE 15
Results for Example 64

| | |
|---|---|
| Yield | 32% |
| Soluble Oligomers of Cured (%) | — |
| Granule Appearance | Off White |
| Total Drying Time (hr.) | ~12.5 |
| Un-cured Volume Weighted Mean* (μm) | 273 |
| Cured - Volume Weighted Mean* (μm) (4-hrs.) | 411 |

*Table Note:
Volume Weighted Mean measured in accordance with the Wet Particle Size & Distribution (in Acid) test method.

Example 65: Sevelamer carbonate dry particles, having a mean particle size of 90 μm, were hydrated with water to a % LOD of approximately 80%. The wet material was wet co-milled with a 813 μm screen at 1300 rpm. After it was co-milled, the sample material was dried in a fluid bed dryer having an inlet air temperature of 65° C. at 30-40 cubic foot per minute (CFM), to about 30% LOD (approximately 70 minutes). It was then co-milled again with a 1575 μm screen at 1300 rpm. The milled material was placed in a dryer manufactered by Littleford Day (model M5) and dried at 110° C. for 4 hours at approximately 2-3% LOD, under a 400 to 500 mbar vacuum with a nitrogen purge, with agitation approximately every 0.5 hour for 30 seconds at 50 rpm. The dried material was sieved through a −20/+50 mesh screen. A portion of the sieved particles was then cured in a dryer manufactered by Littleford Day (model M5) and at 110° C. for 4 hours, under a 400 to 500 mbar vacuum with a nitrogen purge, with agitation approximately every 0.5 hour for 30 seconds at 50 rpm. The measured properties of the sample in both the uncured and cured state are presented in Table 16.

TABLE 16
Results for Example 65

| Yield | | | 66% |
|---|---|---|---|
| Soluble Oligomer of Cured (%) | | | 0.06 |
| Granule Appearance | | | Off White |
| Total Drying Time (hr.) | | | ~9.5 |
| Un-cured Volume Weighted Mean* (μm) | | | 378 |
| Cured - Volume Weighted Mean* (μm) | | | 431 |
| Competitive Phosphate | Uncured | 60 min. | 2.01 |
| Binding | | 120 min. | 1.62 |
| | Cured | 60 min. | 2.13 |
| | | 120 min. | 1.75 |

*Table Note:
Volume Weighted Mean measured in accordance with the Wet Particle Size & Distribution (in Acid) test method.

Example 66: Sevelamer carbonate dry particles, having a mean particle size of 90 μm, were hydrated with water to a % LOD of approximately 40%. The material was placed in a dryer manufactered by Littleford Day (model M5) and was dried at 110° C. for 4.5 hours (approximately 30% LOD), under a 400 to 500 mbar vacuum with a nitrogen purge, with agitation approximately every 0.5 hour for 30 seconds at 50 rpm. It was then co-milled with a 1575 μm screen at 1300 rpm. The milled material was placed in a dryer manufactered by Littleford Day (model M5) at 110° C. for 4 hours at approximately 2-3% LOD, under a 400 to 500 mbar vacuum with a nitrogen purge, with agitation approximately every 0.5 hour for 30 seconds at 50 rpm. The dried material was sieved through a −20/+50 mesh screen. A portion of the sieved particles was then cured in a dryer manufactered by Littleford Day (model M5) at 110° C. for 4 hours, under a 400 to 500 mbar vacuum with a nitrogen purge, with agitation approximately every 0.5 hour for 30 seconds at 50 rpm. The measured properties of the sample in both the uncured and cured state are presented below in Table 17A.

TABLE 17A
Results for Example 66

| Yield | | | 78% |
|---|---|---|---|
| Soluble Oligomer of Cured (%) | | | 0.07 |
| Granule Appearance | | | Off White |
| Total Drying Time (hr.) | | | ~8.5 |
| Un-Cured Volume Weighted Mean* (μm) | | | 473 |
| Cured - Volume Weighted Mean* (μm) (4-hrs.) | | | 487 |
| Competitive Phosphate | Uncured | 60 min. | 1.94 |
| Binding | | 120 min. | 1.61 |
| | Cured | 60 min. | 2.12 |
| | | 120 min. | 1.91 |

*Table Note:
Volume Weighted Mean measured in accordance with the Wet Particle Size & Distribution (in Acid) test method.

Examples 67-74 were prepared in accordance with the method set forth in Example 66. The properties of these examples are presented in Table 17B.

TABLE 17B
Examples 67-74 Test Results

| Volume | |
|---|---|
| Weighted | Competitive Phosphate |
| Mean (or | Binding |

| | | | |
|---|---|---|---|
| Example | APS) (μm) | 60 Min | 120 Min |

CURED PARTICLES

| | | | |
|---|---|---|---|
| 67 | 326 | 1.71 | 1.28 |
| (80° C.) | | | |
| 68 | 500 | 2.17 | 1.64 |
| 69 | 590 | 2.1 | 1.75 |
| 70 | 456 | 2.24 | 1.49 |
| 71 | 495 | 2.06 | 1.55 |

UNCURED PARTICLES

| | | | |
|---|---|---|---|
| 72 | 530 | 2.29 | 1.73 |
| 73 | 445 | 2.47 | 1.66 |
| 74 | 477 | 2.21 | 1.51 |

*Table Note:
Volume Weighted Mean measured in accordance with the Wet Particle Size & Distribution (in Acid) test method. The cured particles of Example 67 - were prepared by drying at 80° C. instead of 110° C.

### Preparation of Sevelamer Carbonate Constituent Particles (Example 75)

Poly(allylamine hydrochloride) aqueous solution (50% w/w, 2200 kg) was charged to a 6300 litre glass lined reactor. Water was added (2585 kg) followed by sodium hydroxide solution (32%, 1052 kg). The solution was distilled under vacuum (<300 mbar) until approximately 10% water was distilled off and make-up water was added up to the original level. The solution was fed to a LIST CoRotating Kneading Reactor (LIST CKR 1000) via a premixing system in which epichlorohydrin was added (0.094 eq) prior to entry into the CKR 1000. The mixture was cross-linked in the CKR 1000 at <80° C., and emerged from the List CKR as a gel. The gel mixture was discharged (337 kg/h) into water (3092/h) to which sodium hydroxide (32%, 120 kg/h) had been added. The resulting free base sevelamer was washed with water to below 50 μS/cm conductivity. The resulting gel slurry was fed (1800 litre/h) to continuous carbonation reactor to which carbon dioxide was added at a rate as to provide approximately 21% carbonate salt. The resulting slurry was spray dried at <85 deg C. with secondary fluid bed drying at <80 deg C. to yield sevelamer carbonate. The sevelamer carbonate was passed through a 50 mesh screen to yield dry particles, having a mean particle size of 90 μm.

### Preparation of Cured Sevelamer Carbonate Aggregate Particles

The sevelamer carbonate dry particles were hydrated with water to a % LOD of approximately 40%. The material was placed in a dryer and dried at 110° C. for 4.5 hours (approximately 30% LOD), under a 400 to 500 mbar vacuum with a nitrogen purge, with agitation approximately every 0.5 hour for 30 seconds at 50 rpm. It was then co-milled with a 1575 μm screen at 1300 rpm. The milled material was placed in a dryer and dried at 110° C. for 4 hours to approximately 2-3% LOD, under a 400 to 500 mbar vacuum with a nitrogen purge, with agitation approximately every 0.5 hour for 30 seconds at 50 rpm. The dried material was sieved through a −20/+50 mesh screen. The sieved particles were then cured in a dryer at 110° C. for 4 hours, under a 400 to 500 mbar vacuum with a nitrogen purge, with agitation approximately every 0.5 hour for 30 seconds at 50 rpm.

TABLE 18
Characterization Data of Cured Sevelamer
Carbonate Aggregate Particles

| | Result | | | |
|---|---|---|---|---|
| Characterization Test | Run 1 | | Run 2 | |
| pH | 9.5 | | 9.5 | |
| Tap Density | 0.47 | g/ml | 0.44 | g/ml |
| Bulk Density | 0.37 | g/ml | 0.36 | g/ml |
| True Density by | 1.25 | g/cm³ | 1.26 | g/cm³ |
| Helium Pycnometry | | | | |
| DSC - Glass Transition | 59.18° | C. | 56.39° | C. |
| Elemental Analysis | C, 55.3%: H, 9.9%; | | C, 54.7%: H, 10.1%; | |
| | N, 18.0%. | | N, 17.9%. | |
| Titratable Amines | 12.6 | | 12.6 | |
| % Carbonate | 16.4% | | 16.4% | |
| % Loss on Drying | 3.1% | | 4.4% | |
| (LOD) | | | | |
| % Soluble Oligomers | 0.03% | | 0.03% | |
| Allylamine (ppm) | 0.69 | ppm | 0.61 | ppm |
| Competitive Phosphate | 2.1 | mmol/g | 2.1 | mmol/g |
| Binding | | | | |
| Dry Particle Size, | 865 | microns | 882 | mcrons |
| Volume Weighted | | | | |
| Mean | | | | |
| Wet Particle Size | 475 | microns | 466 | microns |
| (in Acid), Volume | | | | |
| Weighted Mean | | | | |
| Wet Particle Size | 649 | micron | 644 | micron |
| (in Phosphate Buffer) | | | | |
| Volume Weighted | | | | |
| Mean, pH 3.2 | | | | |

USP Sieve Testing

| | | |
|---|---|---|
| 300 to 850 microns | 91.0% | 90.2% |
| >850 micron | 0.0% | 0.0% |
| <300 micron | 9.0% | 9.8% |

* Table Notes:
Bulk and Tap Densities - were determined using a Vankel Bulk and Tap Density Tester.
True Density - was determined by using a Helium Pycnometry and all measurements were conducted in duplicate.
Sieve Analysis - was conducted according to cUSP <786> using a Restch Sieve Shaker with sieve sizes of 850 and 300 micron.
DSC - Glass Transition - was determined on a TA DSC Q100 using the following temperature program: Equilibrated at −30° C., Heated at 10° C./minute to 200° C., Held for 20 minutes, Cooled at 10° C./minute to −30° C., Equilibrated at −30° C., Heated at 10° C./minute to 350° C.

### Preparation of Tablets of Cured Sevelamer Aggregate Particles

Sevelamer 800 mg tablet core were prepared from a blend consisting of 99.1 g of cured particles anhydrous, 13.5 g of purified water, 12.53 g of microcrystalline cellulose, 0.13 g of colloidal silicon dioxide (CSD), and 0.125 g sodium stearyl fumarate. The required quantities of the cured particles, purified water, colloidal silicon dioxide, microcrystalline cellulose (MCC), and sodium stearyl fumarate were weighed. The cured particles and CSD were mixed in a high shear granulator. While mixing, purified water was added to the cured particles/CSD mix to hydrate the sevelamer carbonate to a moisture content of 12%. The wetted cured particles/CSD mix was sieved through a 1.0 mm opening screen and then mixed with MCC and sodium stearyl fumarate to form a final bend. The final blend was then compressed on a power assisted tablet press at 20 to 40 KN force to give a core tablet with an average weight of 1012.3 mg and with different average tablet hardness namely 374N, 452N, 512N, and 564N. The resulting tablets consisted of 909.1 mg 12% hydrated sevelamer carbonate (equivalent to 800 mg anhydrous sevelamer carbonate), 101.2 mg MCC, 1.0 mg of CSD, and 1.0 mg of sodium stearyl fumarate. The components of the tablet core are presented in Table 19 below.

TABLE 19
800 mg Tablet Core

| | Actual Amount | | Amount in |
|---|---|---|---|
| | of Ingredients | Actual % of | Tablets |
| Ingredient | (g) | Ingredients | (mg) |
| Cured Sevelamer | 99.0 | 79.02 | 800.0 |
| Carbonate Aggregate | | | |
| Partilces | | | |
| Water | 13.5 | 10.78 | 109.1 |
| Colloidal Silicon Dioxide | 0.13 | 0.10 | 1.0 |
| Microcrystalline Cellulose | 12.53 | 10.00 | 101.2 |
| Ceolus KG 1000 | | | |
| Sodium stearyl fumarate | 0.125 | 0.10 | 1.0 |
| Total | 125.3 | 100.00 | 1012.4 |

### Preparation of Coated (Cured Sevelamer Carbonate) Tablets

Compressed core tablets prepared as described above were coated in coating pan with an aqueous coating solution having a solids composition comprising:

TABLE 20
Coating of Tablet Cores

| Material | % w/w |
|---|---|
| Polyvinyl Alcohol Partially Hydrolyzed, cUSP | 52.26 |
| Talc, cUSP | 30.00 |
| Macrogel/Polyethylene Glycol 3350, NF | 14.74 |
| Polysorbrate 80, NF | 3.00 |

* Table Note:
The coating solution was applied to the compressed core tablets until a weight gain of approximately 4 to 6% was achieved.

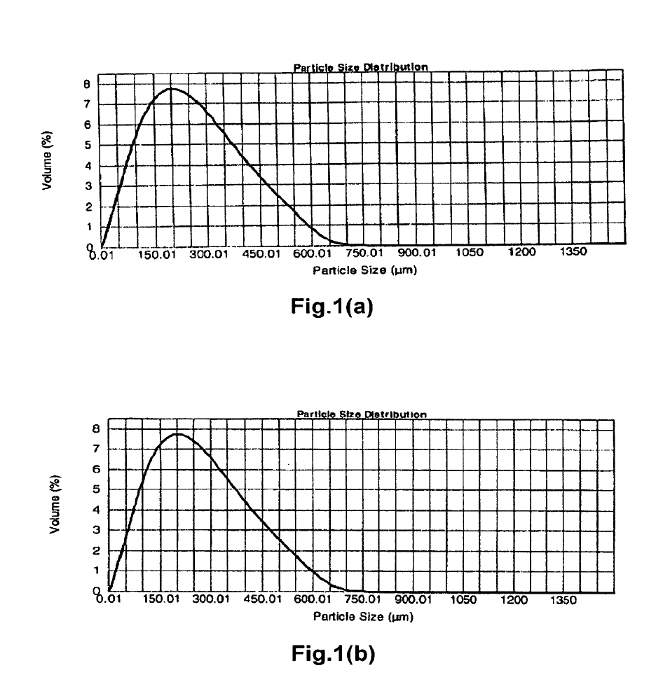

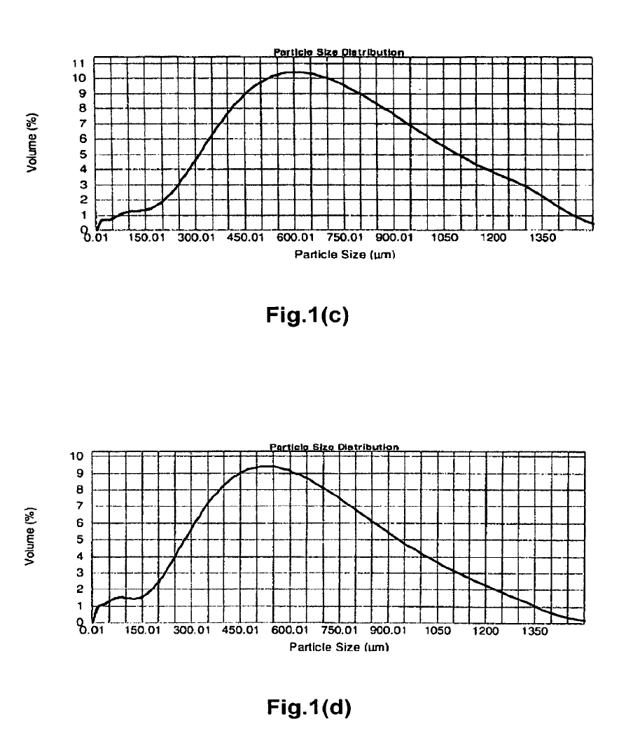

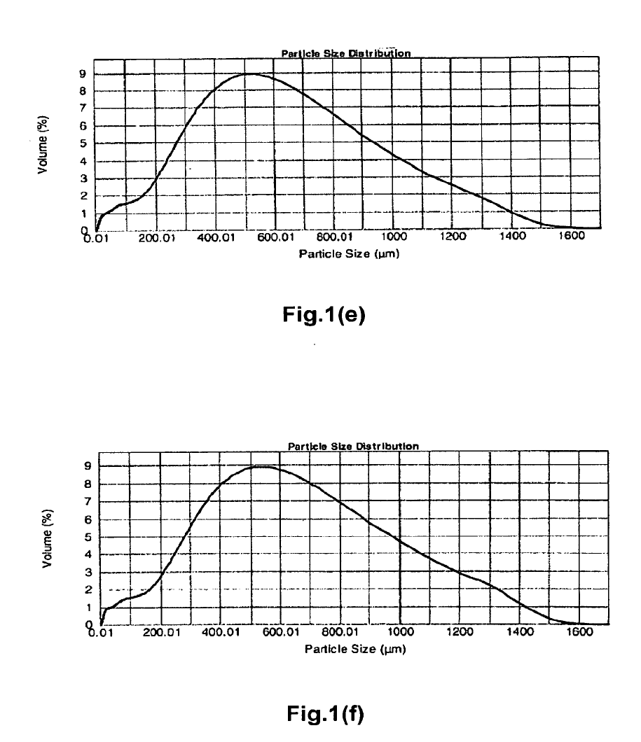

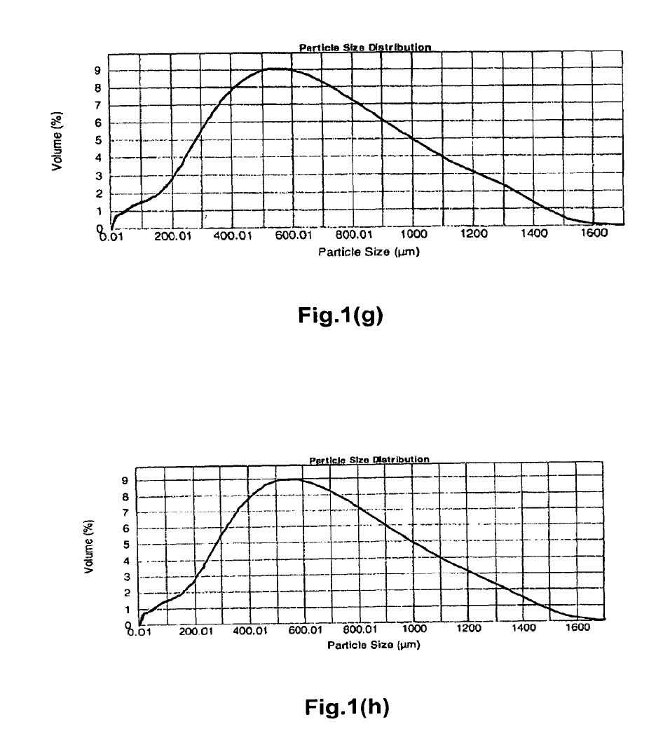

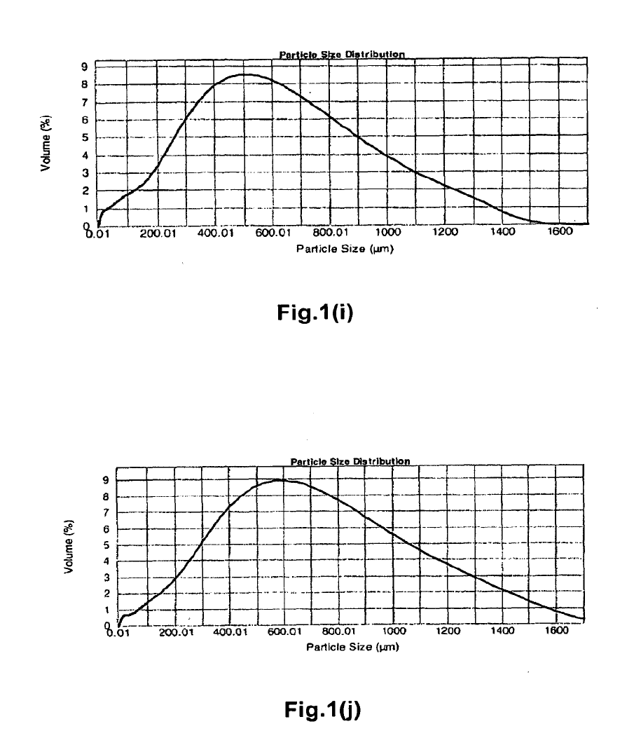

Example 76: A series of tablets, prepared in accordance with Example 75, were tested in accordance with the Tablet Particle Size Dissolution (in Phosphate Buffer) and the results are presented in Table 21 and FIGS. 1A-J.

TABLE 21
Tablet Particle Size Relative to Tablet Compression Strength

| Tablet | Particle Size Distribution | | |
|---|---|---|---|
| Compression | Volume Weighted | | |
| Strength | Mean (μm) | Volume % Mode | FIG. |
| Sevelamer | 182.2 | 175-225 | 1A |
| Carbonate | | | |
| (Control) | | | |
| Sevelamer | 183.2 | 175-225 | 1B |
| Carbonate | | | |
| (Control) | | | |
| 374N | 533.7 | 575-625 | 1C |
| 374N | 448.5 | 500-550 | 1D |
| 452N | 450.3 | 500-550 | 1E |
| 452N | 464.0 | 525-575 | 1F |
| 512N | 479.2 | 525-575 | 1G |
| 512N | 480.8 | 525-575 | 1H |
| 564N | 429.4 | 475-525 | 1I |
| 564N | 513.2 | 550-600 | 1J |

Test Methods

### Non-Competitive Phosphate Binding Capacity

Buffer Preparation: 0.680 g of KH₂PO₄, 10.662 g of morpholinoethane sulfonic acid and 2.338 g of NaCl were weighed into a 500 ml volumetric flask. 300 ml of deionized water and the solids were dissolved. Additional deionized water was added until the total volume of buffer was 500 ml. The pH was adjusted to 5.8 using 1 N NaOH.

Sample Preparation: The percent loss on drying (% LOD) by Thermogravimetric Analyzer (TGA) of 25 mg of each polymer was determined on a Thermogravimetric Analyzer, TA Instruments, Model TGA Q 500, purged with nitrogen and using platinum pans. The following heating conditions were used:
Heating rate: 10° C./min
End temperature: 85° C.
Hold time: 60 minutes

The % LOD was determined as the % weight loss over 65 minutes and the result was used to calculate the target sample weight with the following formula:

Weight=33.35 mg/(1-(LOD/100)).

Binding Procedure: The calculated target sample weight per polymer was weighed into each of two 50 ml plastic sample bottles. A 25 ml aliquot of the 10 mM Phosphate Buffer Solution was transferred into each of the sample bottles. The solutions were mixed well by vortexing and then shaken in an orbital shaker at 37° C. and 250 RPMs for 60 minutes. During shaking it was ensured that the polymer-particles did not adhere to the walls or lid of the sample bottle. After 60 minutes the shaker was stopped and the polymer was allowed to settle. An aliquot of exactly 2.0 ml was taken from each solution. The aliquots were filtered into small vials using a disposable syringe and 25 mm syringe filter and then diluted at a ratio of 1 part solution to 9 parts DI water. The sample bottles were shaken for a further 1 hour (total of 2 hours altogether) and the sampling procedure was repeated. Four phosphate standards were prepared by diluting the 10 mM Phosphate Buffer Solution as follows:

| | Volume of 10 mM | | |
|---|---|---|---|
| Std Conc | Phosphate Solution | Volume of H₂O | Total Volume |
| (mM) | (mL) | (mL) | (mL) |
| 0.30 | 0.75 | 24.25 | 25 |
| 0.50 | 0.50 | 9.50 | 10 |
| 0.75 | 0.75 | 9.25 | 10 |
| 1.00 | 1.00 | 9.00 | 10 |

The standards and samples were analyzed by ion chromatography using a Dionex ICS3000 instrument with conductivity detection. The 0.75 mM Standard was used as a check standard to verify the system suitability by re-injecting this standard after every 6 sample injections. The following instrument conditions were used:
Column: Dionex, AS 11-HC, 4×250 mm,
Guard Column: AG11-HC, 4×50 mm,
Mobile Phase=40 mM KOH (using eluent generator)
Conductivity detector current set at 149 mA
Column Temperature: 30° C.
Flow rate: 1.5 mL/min
   Injection volume: 25 μL
Run time: 6 minutes
Retention time of phosphate: ˜4 mins

A standard curve was prepared and the unbound phosphate (mM) for each test solution was calculated taking into account the 10-fold dilution. The bound phosphate was determined using the following equation:

Bound PO₄ (mmol/g)=[(10−Unbound PO₄)×Vol.×1000]/MassP where: Vol.=volume of test solution (L) MassP=LOD adjusted mass of polymer (mg)
The results from the duplicate analyses were averaged.

### Competitive Phosphate Binding Capacity

Buffer Preparation (10 mM Phosphate Buffer Solution with Acids): 0.680 g of KH₂PO₄, 10.662 g of morpholinoethane sulfonic acid and 2.338 g of NaCl were weighed into a 500 ml volumetric flask. 300 ml of deionized water and the solids were dissolved. Additional deionized water was added until the total volume of buffer was 500 ml. A 10 mL aliquot of this solution was taken and stored for use in the preparation of standards. 3.537 g of Glycochenodeoxycholic acid, sodium salt (“GCDC”)and 2.283 g of oleic acid, sodium salt were added to the remaining 490 ml of buffer solution and the pH was adjusted to pH 5.8 with 1 N NaOH. The solution was well mixed (Note that oleic acid did not dissolve but formed a suspension. It was ensured that the solution was well mixed and the suspended oleic acid was mixed as homogenously as possible before taking aliquots).

Sample Preparation: The % LOD drying was determined as set forth above.

Binding Procedure: The procedure as set forth above was repeated using a 25 ml aliquot of the 10 mM Phosphate Buffer Solution with Acids instead of the 10 mM Phosphate Buffer Solution.

### Determination of Dry Particle Size and Distribution

The dry particle size and distribution of particle sizes was determined as volume % using a Malvern Mastersizer 2000 equipped with a Scirocco 2000 dry powder dispensing unit.

Calibration Sample Preparation: Approximately 200 mg of a 1000 μm microsphere standard was placed into a 14 mL round bottom test. 5mL methanol and 1 small drop of Triton X was added. The mixture was gently stirred and then analyzed in the 0.1 M phosphate buffer by making a triplicate measurement.

The Mastersizer was modified by removing the ball bearings and mesh basket positioned above the venturi from the feed tray and the sample was fed to the machine and the particle size and distribution were determined using the following parameters:
Model: General Purpose
Measurement time: 20 sec
Measurement snaps: 20,000
Background measurement time: 12 sec
Background measurement snaps: 12,000
Obscuration limits: 1 to 10%
Feeding rate: 30%
Dispersion Air pressure: 1.5 bar
Refractive Index: Calibration samples: 1.49; Test samples: 1.54

### Determination of Wet Particle Size and Distribution (Phosphate Buffer or Acid Buffer)

The wet particle size and distribution of particle sizes of the crosslinked polyamine particles was determined as volume% using a Malvern Mastersizer 2000-HydroS cell.

Phosphate Buffer Dispersant Preparation (also referred to as Phosphate Buffer): A 0.1 M solution of phosphoric acid was prepared by adding 680 μl of 85% phosphoric acid into a 100 ml volumetric flask and diluting to volume. A 0.1 M monobasic sodium monophosphate dihydrate solution was prepared by dissolving 15.6 g of NaH₂PO₄·2H₂O in 500 ml deionized water in a 1 L volumetric flask and diluting to volume. The dispersant buffer was prepared by mixing 55 ml of the 0.1 M phosphoric acid with 945 ml of the 0.1 M monobasic sodium monophosphate dihydrate solution.

Calibration Sample Preparations: Approximately 200 mg of a 300 μm microsphere standard was placed into a 14 mL round bottom test. 5mL methanol and 1 small drop of Triton X was added. The mixture was gently stirred and then analyzed in the 0.1 M phosphate buffer by making a triplicate measurement. In addition, approximately 200 mg of a 1000 μm microsphere standard was placed into a 14 mL round bottom test. 5mL methanol and 1 small drop of Triton X was added. The mixture was gently stirred and then analyzed in the 0.1 M phosphate buffer by making a triplicate measurement.

Optional—Test Sample Preparation (in 1N HCL, also referred to Acid or Acid Buffer): Approximately 200 mg of test sample was placed into a 25 mL glass or plastic vial. 5mL of 1N HCL was introduced into the vial and the vial was shaken gently for 2 hours at 200 rpm in an obital shaker. Duplicates of each sample were run.

Optional—Test Sample Preparation (in Phosphate Buffer Dispersant): Approximately 200 mg of test sample was placed into a 25 mL glass or plastic vial. 5 mL of Phosphate Buffer Dispersant was introduced into the vial and the vial was shaken gently for 2 hours at 200 rpm in an obital shaker. Duplicates of each sample were run.

Particle Size Measurement: 150 ml of the dispersant buffer was added to tank of the Malvern Mastersizer 2000-HydroS cell and the start was selected. For each sample, the entire content of the shaken vial was added to the tank and any material remaining in the vial was rinsed from the vial and added to the tank. After two minutes in the tank the sample was measured according to the following parameters and the cell was cleaned by flushing with de-ionized water twice:
Dispersant: Phosphate Buffer, pH 3.2
Refractive Index: Calibration samples: 1.49; Test samples: 1.54
Absorption: 0
Model: General Purpose
Sensitivity: Normal
Measurement time: 20 sec
Measurement snaps: 20,000
Background measurement time: 12 sec
Background measurement snaps: 12,000
Obscuration limits: Calibration samples: 0.5% to 10%; Test Samples: 1% to 10%
Pump/Stirrer speed: Calibration samples: 2200 rpm; Test samples: 1500 rpm
Ultrasonic: Continuous from start; Stabilizing period 15 seconds
Tip displacement: 30%
Tank fill: Automatic
Aliquot: 1
Measurements: 1 per test sample aliquot (one triplicate for each standard;
   100 μm and 300 μm)
Cleaning: After each aliquot
Flush cycles: 2
Cleaning Mode: Automatic, full wash, manual

The Malvern Mastersizer reports, inter alia, the Volume Weighted Mean (μm).

### Tablet Dissolution Particle Size and Distribution Test (Phosphate Buffer or Acid Buffer)

The particle size and distribution of particle sizes of a tablet comprising crosslinked polyamine particles was determined as volume % using a Malvern Mastersizer 2000-HydroS cell.

Phosphate Buffer Dispersant Preparation (also referred to as Phosphate Buffer): A 0.1 M solution of phosphoric acid was prepared by adding 680 μl of 85% phosphoric acid into a 100 ml volumetric flask and diluting to volume. A 0.1 M monobasic sodium monophosphate dihydrate solution was prepared by dissolving 15.6 g of NaH₂PO₄·2H₂O in 500 ml deionized water in a 1 L volumetric flask and diluting to volume. The dispersant buffer was prepared by mixing 55 ml of the 0.1 M phosphoric acid with 945 ml of the 0.1 M monobasic sodium monophosphate dihydrate solution.

Calibration Sample Preparation: Approximately 200 mg of a 300 μm microsphere standard was placed into a 14 mL round bottom test. 5mL methanol and 1 small drop of Triton X was added. The mixture was gently stirred and then analyzed in the 0.1 M phosphate buffer by making a triplicate measurement.

Optional—Test Sample Preparation (in IN HCL): The tablet is cut into quarters using a tablet cutter and a quarter of the tablet was placed into a 25 mL glass or plastic vial. 5mL of IN HCL was introduced into the vial and the vial was shaken gently for 30 minutes at 200 rpm in an obital shaker.

Optional—Test Sample Preparation (in Phosphate Buffer Dispersant): The tablet is cut into quarters using a tablet cutter and a quarter of the tablet was placed into a 25 mL glass or plastic vial. 5mL of Phosphate Buffer Dispersant was introduced into the vial and the vial was shaken gently for 30 minutes at 200 rpm in an obital shaker.

Particle Size Measurement: 150 ml of the Phosphate Buffer was added to the tank of the Malvern Mastersizer 2000-HydroS cell and the start was selected. For each sample, the entire content of the shaken vial was added to the tank and any material remaining in the vial was rinsed from the vial and added to the tank. After two minutes in the tank the sample was measured according to the following parameters and the cell was cleaned by flushing with de-ionized water twice:
Dispersant: Phosphate Buffer, pH 3.2
Dispersant Refractive Index: 1.33
Refractive Index: Calibration samples: 1.49; Test samples: 1.54
Absorption: 0
Model: General Purpose
Sensitivity: Normal
Measurement time: 20 sec
Measurement snaps: 20,000
Background measurement time: 12 sec
Background measurement snaps: 12,000
Obscuration limits: Calibration samples: 0.5% to 10%;
Test Samples: 1% to no lower than 10%
Pump/Stirrer speed: Calibration samples: 2000 rpm; Test samples: 1500 rpm
Ultrasonic: Continuous from start; Stabilizing period 15 seconds
Tip displacement: 30%
Tank fill: Automatic
Aliquot: 1
Measurements: 1 per test sample aliquot (one triplicate for each standard;
   100 μm and 300 μm)
Cleaning: After each aliquot
Flush cycles: 2
Cleaning Mode: Automatic, full wash, manual
The Malvern Mastersizer reports the volume weighted mean for the sample and a graph of the Particle Size Distribution (Volume % versus Particle Size, μm), see for example FIGS. 1A-J. From this graph, the Volume % mode is obtained as the peak on the respective curve.

### Determination of Mean Gray Value Using Bright Field Microscopy

After sieving to a mesh size that is −20/+50, a representative sample of the crosslinked polyamine particles were sieved using a 35 mesh sieve. A representative sample of the particles retained on the sieve was spread over a glass slide. Images having 15-40 particles within the field of view were taken with an Olympus SZX12 Stereomicroscope equipped with an Olympus QColor 5 digital camera and set with the following parameters: 0.5×objective lens, 10×total magnification, bright field setting, and open light filters (FR, LBD and ND25).

Mean Gray Value was determined using Microsuite Biological Suite 2.3 (Build 1121). Image magnification was set at 10× using software calibration. The images were converted from the full color to 8-bit format with 230 colors. Two color phases were used: Phase I (green for the background) was set from color value 0-112, and Phase II (red for the particles) was set from color value 114-250). The minimum particle size used in the analysis was set at 1000 pixels and the fill holes option was selected. A gray value for each pixel in every particle in the image was assigned, and mean individual particle gray value was calculated, by the software. The mean gray value, which represents the arithmetic mean of the individual particles gray value means, was determined for the imaged collection of particles. Two additional representative samples of the particles retained on the 35 mesh sieve were analyzed and the mean gray values for each of the three images were averaged to establish the Mean Gray Value.

### Bile Acid Binding Capacity

After analyzing the competitive phosphate binding of a polymer sample by ion chromatography the bile acid binding capacity of the same samples was analyzed using HPLC according to the following procedure:

Standard Preparation: 0.177 g of GCDC was weighed into a 25 ml volumetric flask and diluted to the mark using a 100 mM morpholinoethane sulfonic acid stock solution to form a 15 mM GCDC stock solution. Four standards having the following concentrations were prepared by diluting the GCDC stock solution in volumetric flasks as follows:

| Standard Conc | Volume 15 mM GCDC stock | Vol. Flask |
|---|---|---|
| (mM) | (μL) | (mL) |
| 1.50 | 1000 | 10 |
| 1.00 | 750 | 10 |
| 0.75 | 500 | 10 |
| 0.48 | 800 | 25 |

A blank was prepared by diluting the MES buffer stock 1-to-10.

For the HPLC determination, the following parameters were used:
Column: Platinum EPS-C18, 33×7 mm, 3 micron, rocket format
MP: A=make up a 15 mM ammonium acetate solution (adjust pH to 5.3 with acetic acid) and mix 800 ml of this solution with 200 ml of acetonitrile solution, to give a 4:1 mixture.
MP: B=acetonitrile
Flow rate: 2 ml/min
Column Temp: 30° C.
Injection Volume: 10 μl
UV Detection: 210 nm
with the use of the following gradient:

| Time (minutes) | % B |
|---|---|
| 0 | 20 |
| 2 | 20 |
| 4 | 95 |

with stop run=4.0 minutes and post run=2.5 minutes.

The following injection format was used: blank twice, standards twice, blank, then test samples once each with the 1.0 mM standard injected after every 9 sample injections for system suitability testing. The system was suitable if the difference between the original standards and the suitability standard is less than 5%.

A standard curve was set up and the unbound GCDC (mM) for each test solution was calculated. The bound GCDC was determined using the following equation:

Bound GCDC (mmol/g)=[(15−Unbound GCDC)×Vol.×1000]/MassP where: Vol.=volume of test solution (L) and MassP=LOD adjusted mass of polymer (mg).

### Crosslinked Amine PolymerUrinary Phosphorous Reduction (In Vivo-Rats)

House male Sprague Dawley (SD) rats were used for the experiments. The rats were placed singly in wire-bottom cages, fed with Purina 5002 diet, and allowed to acclimate for at least 5 days prior to experimental use.

To establish baseline phosphorus excretion, the rats were placed in metabolic cages for 48 hours. Their urine was collected and its phosphorus content analyzed with a Hitachi analyzer to determine phosphorus excretion in mg/day. Any rats with outlying values were excluded; and the remainder of the rats were distributed into groups.

Purina 5002 was used as the standard diet. The crosslinked amine polymer being tested in each group was mixed with Purina 5002 to result in the desired final crosslinked amine polymer concentration for each group. Cellulose at 0.5% by weight was used as a negative control. For each rat, 200g of diet is prepared.

Each rat was weighed and placed on the standard diet. After 4 days the standard diet was replaced with the treatment diet (or control diet for the control group). On days 5 and 6, urine samples from the rats at 24 hours (+/−30 minutes) were collected and analyzed. The test rats were again weighed, and any weight loss or gain was calculated. Any remaining food was also weighed to calculate the amount of food consumed per day. A change in phosphorus excretion relative to cellulose negative control was calculated. Percentage reduction of urinary phosphorous was determined using the following equation: % Reduction of Urinary Phosphorous=[(urinary phosphorous of negative control (mg/day) urinary phosphorous of experimental (mg/day))/urinary phosphorous of negative control (mg/day)]×100.

### Crosslinked Amine Polymer Fecal Bile Acid Increase (In Vivo-Rats)

House male Sprague Dawley (SD) rats were used for the experiments. The rats were placed singly in wire-bottom cages, fed with Purina 5002 diet, and allowed to acclimate for at least 5 days prior to experimental use.

After acclimatization, the rats were split into test groups with 6 rats per group. Purina 5002 with NaH₂PO₄ at a concentration of 0.4 wt % phosphate added was used as the standard diet. The crosslinked amine polymer being tested in each group was mixed with the standard diet to result in the desired final crosslinked amine polymer concentration for each group. Cellulose at 4.0% by weight was used as a negative control.

Each rat was weighed and placed on its respective treatment diet. On day six, the rats were placed in metabolism cages specifically designed to separate and collect fecal material for 24 hours. The fecal material was collected, freeze dried, weighed and ground into a powder. 500 mgs of the powder was added to an extraction vessel and heated to 100° C. at 1500 psi for 10 minutes in an extraction solvent consisting of 80% methanol/20% 500 mM KOH. 250 μls of the extract was evaporated in a speed vac at 45° C. for 2 hours and then was reconstituted in a 50% mixture of calf serum and saline. The bile acid concentration was then quantitated using a Total Bile Acids colorometric assay available from Diazyme Laboratories, Inc. at catalog number DZ092A.

A change in fecal bile acid excretion relative to the cellulose negative control was calculated. Percentage increase of fecal bile acid was determined using the following equation: % Increase in Fecal Bile Acid=[(Fecal Bile Acid of experimental (mg/day)−Fecal Bile Acid of negative control (mg/day))/Fecal Bile Acid of negative control (mg/day))×100.

In-Process Swelling Ratio (ml/g)

The in-process swelling ratio (SR) of polymers may be determined by the following equation:

SR=(weight of wet gel (g)−weight of dry polymer (g))/weight of dry polymer (g).

All publications and patent applications mentioned in this specification are herein incorporated by reference to the same extent as if each individual publication or patent application was specifically and individually indicated to be incorporated by reference.

While preferred embodiments of the present invention have been shown and described herein, it will be obvious to those skilled in the art that such embodiments are provided by way of example only. It is intended that the following claims define the scope of the invention and that methods and structures within the scope of these claims and their equivalents be covered thereby.

1-34. (canceled)

35. A method of manufacturing a pharmaceutical composition comprising:
a) neutralizing or partially neutralizing polyallylamine hydrochloride;
b) crosslinking said polyallylamine hydrochloride with 8-11 wt. % epichlorohydrin;
c) forming constituent particles of crosslinked polyallylamine hydrochloride, having a particle size dso value of between 50 μm and 400 μm by wet milling;
d) carbonating said washed and/or neutralized particles;
e) drying said carbonated particles;
f) hydrating said carbonated particles;
g) re-drying said carbonated particles; and
h) grinding and/or sieving said re-dried particles to form crosslinked amine polymer particles having a dso value of between 675 μm and 1000 μm.

36. A method of manufacturing a pharmaceutical composition according to claim 35, wherein said crosslinked amine polymer particles are cured by exposing the particles to an elevated temperature for more than 1 hour, after the particles have been dried to less than 3% LOD.

37. A method of manufacturing a pharmaceutical composition according to claim 35, wherein said crosslinked amine polymer particles are cured by exposing the particles to an elevated temperature of between 90° C. and 120° C. for between 3 and 6 hours, after the particles have been dried to less than 2% LOD.

38. A method of manufacturing a tablet having greater than 75 wt. % of a pharmaceutical composition comprising a crosslinked amine polymer comprising repeat units represented by the following Formula I and/or Formula II: 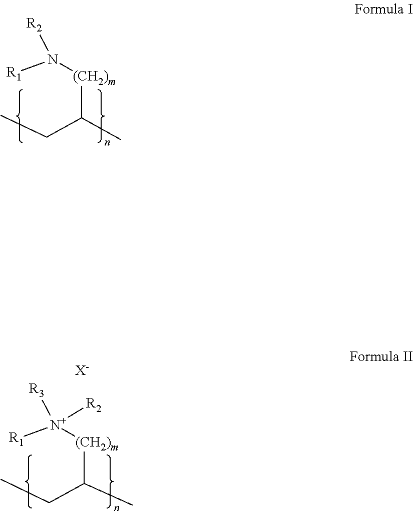
wherein m is an integer from 0 to 2; n is an integer and each R₁, R₂ and R₃ independently represent hydrogen; substituted or unsubstituted, branched or unbranched C₁-C₆ alkyl;
substituted or unsubstituted, branched or unbranched C₁-C₆ alkylamino; X⁻ is a pharmaceutically acceptable counter ion; and a crosslinking agent or residue thereof;
wherein said crosslinked amine polymer comprises particles having a dry particle size distribution wherein greater than 10 vol. % of the particles have a particle size larger than 500 μm; wherein said crosslinked amine polymer particles are cured by exposing the particles to an elevated temperature for more than 1 hour, after the particles have been dried to less than 3% LOD.

39. A method of manufacturing a tablet according to having greater than 75 wt. % of a pharmaceutical composition comprising a crosslinked amine polymer comprising repeat units represented by the following Formula I and/or Formula II: 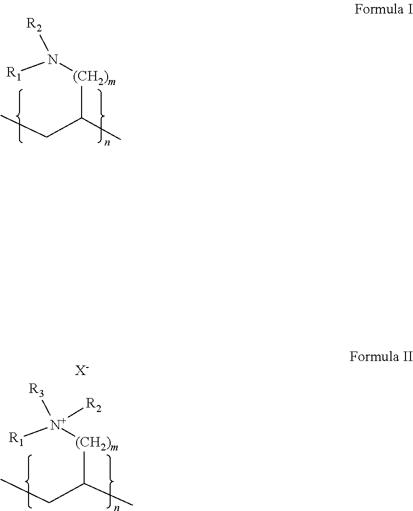
wherein m is an integer from 0 to 2; n is an integer and each R₁, R₂ and R₃ independently represent hydrogen; substituted or unsubstituted, branched or unbranched C₁-C₆ alkyl;
substituted or unsubstituted, branched or unbranched C₁-C₆ alkylamino; X⁻ is a pharmaceutically acceptable counter ion; and a crosslinking agent or residue thereof;
wherein said crosslinked amine polymer comprises particles having a dry particle size distribution wherein greater than 10 vol. % of the particles have a particle size larger than 500 μm; wherein said crosslinked amine polymer particles are cured by exposing the particles to an elevated temperature of between 90° C. and 120° C. for between 3 and 6 hours, after the particles have been dried to less than 2% LOD.
